(19) United States
(12) Patent Application Publication
(10) Pub. No.: US 2024/0218073 A1
(43) Pub. Date: Jul. 4, 2024

(54) IMMUNOMODULATORY ANTI-CD73 ANTIBODIES AND USES THEREOF

(71) Applicant: Corvus Pharmaceuticals, Inc., Burlingame, CA (US)

(72) Inventors: Andrew Hotson, Burlingame, CA (US); Stephen Willingham, Sunnyvale, CA (US); Richard A. Miller, Portola Valley, CA (US)

(21) Appl. No.: 17/772,092

(22) PCT Filed: Nov. 2, 2020
(86) PCT No.: PCT/US2020/058555

§ 371 Date: Dec. 9, 2022

Related U.S. Application Data
(60) Provisional application No. 62/929,650, filed on Nov. 1, 2019.
(60) Provisional application No. 63/014,015, filed on Apr. 22, 2020.
(60) Provisional application No. 63/078,792, filed on Sep. 15, 2020.

(51) Int. Cl. C07K 16/28; A61K 39/00; A61P 31/14

(52) U.S. Cl. CPC C07K 16/2896; A61P 31/14; A61K 39/00; C07K 2317/76; C07K 2317/92

(57) ABSTRACT

The anti-CD73 antibodies, compositions, and methods provided herein allow, inter alia, for the treatment of cancer and infectious diseases (e.g., bacterial, viral, fungal, parasitic), and for the generation of antigen-specific antibodies in vivo in a subject. Applicants have surprisingly found that administration of an anti-CD73 antibody (e.g., CPI-006) to a subject induces differentiation of B cells and robust expansion of specific B cell clones in the subject. The methods and compositions provided herein are, inter alia, useful for a variety of applications, including personalized medicine.

### CROSS-REFERENCE TO RELATED APPLICATIONS

This application claims priority to U.S. Application No. 63/078,792 filed Sep. 15, 2020; U.S. Application No. 63/014,015 filed Apr. 22, 2020; and U.S. Application No. 62/929,650 filed Nov. 1, 2019; the disclosures of which are incorporated by reference herein in their entirety.

### REFERENCE TO A “SEQUENCE LISTING,” A TABLE, OR A COMPUTER PROGRAM LISTING APPENDIX SUBMITTED AS AN ASCII FILE

The Sequence Listing written in file 048517-547001WO_SequenceListing_ST25, created Oct. 28, 2020, 12,925 bytes, machine format IBM-PC, MS Windows operating system, is hereby incorporated by reference.

### BACKGROUND

New cancer immunotherapies are rapidly entering the clinic due to their promising potency. By targeting surface antigens expressed on tumor cells, monoclonal antibodies have demonstrated efficacy as cancer therapeutics. The immunotherapeutic antibodies may target tumor cells by engaging surface antigens differentially expressed in cancers and invoke tumor cell death by blocking ligand-receptor growth and survival pathways. Further, anti-tumor antibodies can act through mechanisms that engage their Fc portion resulting in antibody-dependent cellular cytotoxicity (ADCC), complement-mediated cytotoxicity (CMC) or antibody-dependent cellular phagocytosis (ADCP). However, due to the frequent mutation rate of genes expressed by tumor cells, the surface expression of the antigens recognized by immunotherapeutic antibodies changes rapidly, which results in patients that do not respond to immunotherapeutic treatment and many that do, relapse. The present invention addresses these and other needs in the art.

### BRIEF SUMMARY

Provided herein are methods of treating an infectious disease in a patient in need thereof by administering to the patient an effective amount of an anti-CD73 antibody including an 1E9 CDR L1, an 1E9 CDR L2, an 1E9 CDR L3, an 1E9 CDR H1, an 1E9 CDR H2, and an 1E9 CDR H3. In embodiments, the infectious disease is a bacterial infectious disease. In embodiments, the infectious disease is a fungal infectious disease. In embodiments, the infectious disease is a viral infectious disease. In embodiments, the infectious disease is a parasitic infectious disease. In embodiments, the viral infection is SARS-CoV, SARS-CoV-1, SARS-CoV-2, MERS-CoV. In embodiments, the viral disease is COVID-19 or MERS.

Provided herein are methods of producing a cancer antigen-binding antibody by (i) administering to a cancer subject an effective amount of an anti-CD73 antibody including an 1E9 CDR L1, an 1E9 CDR L2, an 1E9 CDR L3, an 1E9 CDR H1, an 1E9 CDR H2, and an 1E9 CDR H3, (ii) isolating from the cancer subject a B cell expressing a cancer antigen-binding antibody, and (iii) expressing the gene encoding the cancer antigen-binding antibody, thereby producing a cancer antigen-binding antibody. In embodiments, the gene encoding the cancer antigen-binding antibody is obtained from the isolated B cell in step (ii). In embodiments, the methods comprise isolating the gene from the B cell. In embodiments, the methods comprise isolating the gene from the B cell, and then expressing the gene encoding the cancer antigen-binding antibody.

Provided herein are methods of producing an infectious disease antigen-binding antibody by (i) administering to an infectious disease subject an effective amount of an anti-CD73 antibody including an 1E9 CDR L1, an 1E9 CDR L2, an 1E9 CDR L3, an 1E9 CDR H1, an 1E9 CDR H2, and an 1E9 CDR H3, (ii) isolating from the infectious disease subject a B cell expressing an infectious disease antigen-binding antibody, and (iii) expressing the gene encoding the infectious disease antigen-binding antibody, thereby producing an infectious disease antigen-binding antibody. In embodiments, the gene encoding the infectious disease antigen-binding antibody is obtained from the isolated B cell in step (ii). In embodiments, the methods comprise isolating the gene from the B cell. In embodiments, the methods comprise isolating the gene from the B cell, and then expressing the gene encoding the infectious disease antigen-binding antibody.

Provided herein are methods of treating cancer in a subject in need thereof by administering to the subject an effective amount of a cancer antigen-binding antibody formed by the methods provided herein, including embodiments thereof. In another aspect is provided a method of treating an infectious disease in a subject in need thereof. The method includes administering to the subject an effective amount of an infectious disease antigen-binding antibody formed by the methods provided herein, including embodiments thereof. The infectious disease may be a viral infectious disease, a bacterial infectious disease, a fungal infectious disease, or a parasitic infectious disease.

Provided herein are methods of treating cancer by (i) administering to a first cancer subject an effective amount of an anti-CD73 antibody comprising an 1E9 CDR L1, an 1E9 CDR L2, an 1E9 CDR L3, an 1E9 CDR H1, an 1E9 CDR H2, and an 1E9 CDR H3, (ii) isolating from the first cancer subject a B cell expressing a cancer antigen-binding antibody, (iii) expressing the gene encoding the cancer antigen-binding antibody, thereby forming an isolated cancer antigen-binding antibody; and (iv) administering the isolated cancer antigen-binding antibody to a second cancer subject, thereby treating cancer in a subject. In embodiments, the gene encoding the cancer antigen-binding antibody is obtained from the isolated B cell in step (ii). In embodiments, the methods comprise isolating the gene from the B cell. In embodiments, the methods comprise isolating the gene from the B cell, and then expressing the gene encoding the cancer antigen-binding antibody. Thus, the antibody is formed in the first subject, isolated from the first subject, and then administered to a different subject expressing the same cancer antigen.

Provided herein are methods of treating an infectious disease by (i) administering to a first infectious disease subject an effective amount of an anti-CD73 antibody comprising an 1E9 CDR L1, an 1E9 CDR L2, an 1E9 CDR L3, an 1E9 CDR H1, an 1E9 CDR H2, and an 1E9 CDR H3, (ii) isolating from the first infectious disease subject a B cell expressing an infectious disease-binding antibody, (iii) expressing the gene encoding the infectious disease-binding antibody, thereby forming an isolated infectious disease-binding antibody, and (iv) administering the isolated infectious disease-binding antibody to a second infectious disease subject, thereby treating an infectious disease in a subject. In embodiments, the gene encoding the infectious disease antigen-binding antibody is obtained from the isolated B cell in step (ii). In embodiments, the methods comprise isolating the gene from the B cell. In embodiments, the methods comprise isolating the gene from the B cell, and then expressing the gene encoding the infectious disease antigen-binding antibody. Thus, the antibody is formed in the first subject, isolated from the first subject, and then administered to a different subject having the same infectious disease. In embodiments, the infectious disease is a viral, bacterial, a fungal infection, or a parasitic infection.

Provided herein are methods of detecting a cancer antigen by (i) contacting a cancer antigen with a cancer antigen-binding antibody formed by the methods provided herein including embodiments thereof, and (ii) detecting binding of the cancer antigen-binding antibody to the cancer antigen, thereby detecting a cancer antigen.

Provided herein are methods of detecting an infectious disease antigen by (i) contacting an infectious disease antigen with an infectious disease antigen-binding antibody formed by the methods provided herein including embodiments thereof, and (ii) detecting binding of the infectious disease antigen-binding antibody to the infectious disease antigen, thereby detecting an infectious disease antigen.

These and other embodiments and aspects of the disclosure are provided in more detail herein.

### BRIEF DESCRIPTION OF THE DRAWINGS

FIG. 1 shows clonal abundance plots of B cell clones. B cell receptor sequencing of the CDR3 region was performed to identify the frequency of B cell clones in peripheral blood samples collected pre-treatment and on-treatment at 6 weeks. Expansion of B cell clones was observed after 6 weeks in 7 of 11 evaluated patients. Clonal abundance plots are shown for patients treated with 6 mg/kg CPI-006 (left panel), 6 mg/kg CPI-006+ ciforadenant (center panel), and 12 mg/kg CPI-006 (right panel). Ciforadenant is also known as CPI-444.

FIG. 2 shows clonal abundance plots of B cell clones. B cell receptor sequencing of the CDR3 region was performed to identify the frequency of B cell clones in peripheral blood samples collected pre-treatment and on-treatment at 6 weeks. Cancer type, treatment, and dosage for each patient are as indicated above each clonal abundance plot.

FIGS. 3A-3B illustrate an increase in memory B cells in returning lymphocytes in patients treated with CPI-006. Peripheral blood was collected from patients treated with CPI-006 dosed at 6 mg/kg or greater as a single agent or in combination with ciforadenant at pre-treatment, 0.5 hr post-treatment, or 21 days post-treatment. FIG. 3A shows flow cytometry analysis of blood was stained with lineage specific markers. FIG. 3B shows the fold change in the proportion of class-switched memory B cells (CD27+IgD−) as a percentage of all B cells (CD19+CD20+) at 0.5 hr and 21 days.

FIG. 4 shows analysis of circulating immunoglobulins in serum. Blood was collected from patients treated with CPI-006 for immunoglobulins analysis at pre-treatment or prior to dose administration at the beginning of Cycle 4 (9 weeks), Cycle 7 (18 weeks) and Cycle 10 (27 weeks). No consistent change in circulating Ig levels was observed.

FIGS. 5A-5B show B cell HLA-DR increase in CD73⁺ cells. Peripheral blood mononuclear cells were isolated from patients treated with CPI-006 as a single agent or in combination with ciforadenant at pre-treatment, or 7, 14 or 21 days post-treatment. FIG. 5A show flow cytometry analysis of blood was stained with lineage specific markers. The median fluorescence intensity of HLA-DR was determined for CD73⁺ B cells, and for CD73⁻ B cells.

FIG. 5B shows the ratio of CD73 expression in CD73⁺ B cells to CD73⁻ B cells. The ratio was calculated at each time point for each patient, with an increase in HLA-DR expression specific to CD73⁺ B cells observed in 5 of 6 patients evaluated at Day 7.

FIG. 6 are bar graphs showing CPI-006 directly activates B lymphocytes. Purified B cells were incubated overnight with 10 μg/mL human IgG1 isotype control or CPI-006 or anti-IgM microbeads, a positive control for BCR stimulation. Expression of activation markers CD69, CD83, CD86, CD25 or MHC-II was measured by flow cytometry. Mean fluorescence intensity (MFI) was determined for each activation marker and was normalized to the untreated condition for each donor. The figure legend from top to bottom is representative of each group of four bars from left to right. (N=3-5 donors).

FIG. 7 illustrates expression of CD69 on B cells (CD19+CD3−) as measured by flow cytometry and reported by MFI. Human PBMCs were incubated overnight with 10 μg/mL CPI-006 with or without NECA over a range of concentrations or 10 μM APCP. Expression of CD69 on B cells (CD19+CD3−) was measured by flow cytometry and MFI is reported. Error bars represent mean±SD. *p<0.05, **p<0.01 as determined by t-test.

FIG. 8 are representative microscopy images of human B cells. Cells were treated with CPI-006 or hIgG1 isotype control (top row). Stimulation of B cells through IgM represents a known method for activating B cells and promoting differentiation. Anti-IgM, along with an appropriate isotype control, were therefore included as a positive control (bottom row). Images were collected 48 hours after treatment after cells were cytospun onto slides and Wright Giemsa staining. Data are representative of 3 independent donors.

FIG. 9 is a bar graph showing CPI-006 induces antibody class switching to IgG. 5000 purified human B cells were stimulated with the indicated concentration of CPI-006 or an isotype control. IgG levels were quantified in the culture supernatant after a 7 day incubation by ELISA using an anti-human IgGλ secondary antibody for detection. For each group of four bars, data from left to right represent: isotype control, 0.1 ug/mL CPI-006, isotype control, and 1.0 ug/mL CPI-006.

FIG. 10 is a bar graph showing CPI-006 induces antibody class switching. Human B cells were stimulated in vitro with either CPI-006 (1 ug/ml) or an isotype control for 7 days. IgG and IgM levels were quantified in the culture supernatant after a 7 day incubation by ELISA using an anti-human IgG or anti-human IgM secondary antibody for detection. Antibody concentrations were determined and normalized to the isotype control for each individual donor. N=5 donors shown. Data are represented as mean±SEM of 6 independent experiments, each in duplicate.

FIG. 11 is a bar graph showing CPI-006 decreases IgD and increases CD27. Human B cells were stimulated in vitro with CPI-006 or CPX-016 (1 ug/ml) or an isotype control for 3 days. IgD and CD27 expression were evaluated by flow cytometry. The mean fluorescence intensity (MFI) of each marker was determined in FlowJo and normalized to CPX-016. N=3 donors shown. For each group of two bars, left bar represents treatment with CPI-016 and right bar represents treatment with CPX-006.

FIGS. 12A-12D show that CPI-006 blocks CD73 enzymatic activity and restores T cell proliferation and cytokine production in an AMP-mediated immunosuppressive environment.

FIG. 12A: CD73 catalytic activity was measured with MDA-MB-231 cells or MDA-MB-231 CD73 CRISPR cells in the presence of CPI-006, oleclumab, or 1 mM APCP by adding 250 μM AMP and measuring phosphate levels in the cell culture supernatant. FIG. 12B: Human PBMCs were incubated with AMP in the presence of CPI-006, oleclumab, or isotype control antibody at the saturating concentration of 300 nM. After 4 hours of incubation, the AMP remaining in the supernatant was quantified using the CellTiterGlo assay as described in the Methods. Percentage of inhibition was plotted. Data represent 9 individual donors and error bars represent mean±SD. ****p<0.0001 as determined by t-test. FIG. 12C: Human PBMCs were activated with anti-CD3/anti-CD28 in the presence of AMP and a range of doses of CPI-006, oleclumab, or isotype control antibody. T cell proliferation was assessed by flow cytometry. T cell proliferation for 13 donors treated with 500 nM CPI-006 or oleclumab or 890 nM isotype control. Error bars represent mean±SD. *p<0.05, ***p<0.001, and ****p<0.0001 as determined by t-test. FIG. 12D: Human PBMCs were activated with anti-CD3/anti-CD28 in the presence of AMP and a range of doses of CPI-006, oleclumab, or isotype control antibody. After 4 days in culture, IFNg levels in the supernatant were measured by AlphaLISA (Perkin Elmer). IFNg production for 6 donors treated as described in panel. Error bars represent mean±SD. *p<0.05 and **p<0.01 as determined by t-test.

FIGS. 13A-13E show that CPI-006 directly activates B lymphocytes and induces maturation into antibody secreting plasmablasts in vitro. FIG. 13A: Purified B cells from 3-5 healthy donors were incubated overnight with human IgG1 isotype control or CPI-006 (10 μg/mL) or anti-IgM microbeads, a positive control for BCR stimulation. Expression of activation markers CD69, CD83, CD86, or MHC-II was measured by flow cytometry. FIG. 13B: Time dependent increases in the expression of CD27, IgG, IgM, CD38, and CD138 on purified B cells cultured in the presence of CPI-006 or isotype control (1 μg/mL). Mean fluorescence intensity (MFI) was determined for each marker and was normalized to the untreated or isotype control for each donor. FIG. 13C: B cell activation is unique to CPI-006 as other anti-CD73 antibodies do not induce CD69 expression. Expression of CD69 (MFI) was measured by flow cytometry. FIG. 13D: Purified B cells were incubated overnight with 10 μg/mL human IgG1 isotype control or CPI-006 or equimolar CPI-006 Fab. CD69 and CD73 were measured on B cells by flow cytometry. FIG. 13E: Human PBMCs were incubated overnight with a fixed concentration of CPI-006 (10 μg/mL) along with ibrutinib or vehicle control over a range of concentrations. Expression of CD69 on B cells (CD19^{POS}CD3^{NEG}) was measured by flow cytometry.

FIGS. 14A-14D show that CPI-006 activates human B cells to secrete immunoglobulin and cytokines associated with B cell activation. Purified B cells were incubated for 5 days with 10 μg/mL human IgG1 isotype control or CPI-006 or anti-IgM microbeads, a positive control for BCR stimulation. Concentration of CCL2 (FIG. 14A), CCL3 (FIG. 14B), CCL4 (FIG. 14C), and CCL22 (FIG. 14D) in supernatant were measured by ELISA. Data are represented as mean±SD of 4 independent experiments, each in duplicate.

FIGS. 15A-15K show the B and T cell dynamics in advanced cancer patients treated with CPI-006. FIGS. 15A-15B: CPI-006 induces rapid change in peripheral B cells, returning to baseline by Day 21. FIG. 15C-15H: An increased frequency of memory B cells is observed at doses ≥3 mg/kg. Each symbol represents a treated patient, with lines connecting paired samples when appropriate. Some of these patients received an adenosine A2A receptor antagonist, ciforadenant, in combination with CPI-006. Studies with ciforadenant monotherapy reveal no changes in B cell numbers or immunophenotype. FIG. 15I-15K: Representative examples of BCR repertoire diversification in 3 cancer patients 21 days after CPI-006 treatment.

FIGS. 16A-16C show the anti-SARS-CoV-2 antibody responses in COVID-19 patients treated with CPI-006. FIGS. 16A-16B: Endpoint IgG and IgM titers directed to trimeric spike protein in eight COVID-19 patients treated with CPI-006. FIG. 16C: Inhibitory dilution required to block 50% (ID50) RBD binding to recombinant human ACE2. Each symbol represents an individual patient. Open symbols represent patients dosed with 0.3 mg/kg CPI-006. Filled symbols represent patients dosed with 1 mg/kg CPI-006.

FIGS. 17A-17D show endpoint titers of IgG and IgM directed to Spike and RBD in COVID-19 patients treated with CPI-006. FIGS. 17A-17B: IgG and IgM titers directed to recombinant trimeric spike protein. FIGS. 17C-17D: IgG and IgM titers directed to recombinant RBD. All titers were measured by ELISA. Each symbol represents an individual patient. Red line represents geometric mean of titers in each time point. Open symbols represent patients dosed with 0.3 mg/kg CPI-006. Filled black symbols represent patients dosed with 1 mg/kg CPI-006. Grey filled circles represent serum from untreated convalescent control individuals. P values between each time point and convalescent control group were determined with unpaired Welch's t-test of logarithm transformation of antibody titer values. All the statistical analyses were performed by GraphPad Prism 7. * p<0.05; ** p<0.01.

FIGS. 18A-18E show that CPI-006 induces an increase in memory B cells and memory/effector T cells in COVID-19 patients. FIG. 18A: Frequency of memory B cells (CD19^{POS}IgD^{NEG}CD27^{POS}) within CD19^{POS} gate at baseline and 28 days after treatment in two patients treated with 0.3 mg/kg CPI-006. A third patient treated with 0.3 mg/kg CPI-006 was also evaluated but had insufficient CD19^{POS} cells detectable at day 28 and was therefore excluded from the analysis. FIGS. 18B-18C: Increased frequency of memory/effector CD4^{POS} and CD8^{POS} T cells after CPI-006 treatment. Memory/effector population was defined as CD3^{POS}CD45RA^{NEG}. FIGS. 18D-18E: Serial evaluation of PBMCs from one patient treated with 0.3 mg/kg CPI-006 for ability to secrete IL-2 and IFNg specifically in response to SARS-CoV-2 peptides but not control peptides derived from the HIV GAG protein. S peptide=Peptides derived from SARS-CoV-2 spike protein. MNS=Peptides derived from SARS-CoV-2 membrane, nucleocapsid, and spike proteins.

### DETAILED DESCRIPTION

### Definitions

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used. The following definitions are provided to facilitate understanding of certain terms used frequently herein and are not meant to limit the scope of the present disclosure.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the intemucleotide linkages in DNA are phosphodiester, phosphodiester derivatives, or a combination of both.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

The term “expressing” as provided herein refers to the process of transcribing transcription and/or translating a gene of interest (e.g., a gene encoding a caner antigen-binding antibody or a gene encoding an infectious disease antigen-binding antibody). Thus, the process of expressing a gene results in the formation of the protein encoded by the gene of interest or the mRNA encoding the protein. The transcription and/or translation of the gene of interest may occur in a reaction vessel with the necessary nucleic acids, enzymes, salts and buffers present or it may occur in a cell (e.g., in cell culture, or in a multicellular organism).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, and electroporation. In embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and β-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., an antibody construct or antigen binding domain provided herein) in other proteins with different numbering systems. For example, by performing a simple sequence alignment with a protein (e.g., an antibody construct or antigen binding domain provided herein) the identity and location of residues corresponding to specific positions of the protein are identified in other protein sequences aligning to the protein. For example, a selected residue in a selected antibody construct (or antigen binding domain) corresponds to light chain threonine at Kabat position 40, when the selected residue occupies the same essential spatial or other structural relationship as a light chain threonine at Kabat position 40. In embodiments, where a selected protein is aligned for maximum homology with the light chain of an antibody (or antigen binding domain), the position in the aligned selected protein aligning with threonine 40 is the to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is the to correspond to the threonine 40 residue.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and β-phosphoserine. In embodiments, the amino acid side chain may be a non-natural amino acid side chain. In embodiments, the amino acid side chain is H,

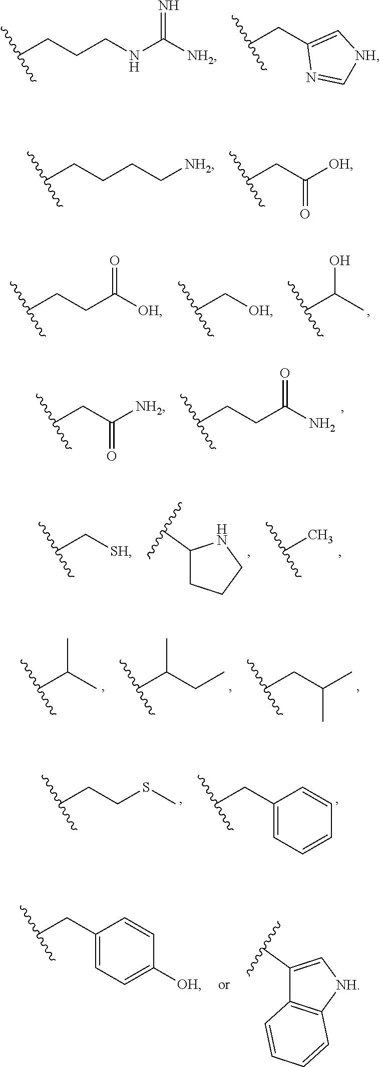

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutyric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]-hept-5-ene-2-carboxylic acid hydrochloride, cis-2-aminocycloheptanecarboxylic acid hydrochloride, cis-6-amino-3-cyclohexene-1-carboxylic acid hydrochloride, cis-2-amino-2-methylcyclohexanecarboxylic acid hydrochloride, cis-2-amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)-acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]-acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-3-(2-thienyl)-Ala-OH, and 4-(hydroxymethyl)-D-phenylalanine.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “peptidyl”, “peptidyl moiety” or “peptide moiety” refers to a monovalent peptide and may be alternatively referred to as a portion of a peptide or a portion of a polypeptide.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source Similarly, a heterologous protein indicates that the protein comprises two or more subsequences that are not found in the same relationship to each other in nature (e.g., a fusion protein).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences described herein or individual domains of the polypeptides described herein), when compared and aligned for maximum correspondence over a comparison window, or designated region as measured using one of the following sequence comparison algorithms or by manual alignment and visual inspection. Such sequences are then the to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains of an antibody or fragment thereof. Non-limiting examples of antibody variants include single-domain antibodies or nanobodies, affibodies (polypeptides smaller than monoclonal antibodies (e.g., about 6kDA) and capable of binding antigens with high affinity and imitating monoclonal antibodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “nanobody” or “single domain antibody” as described herein is commonly well known in the art and refers to an antibody fragment consisting of a single monomeric variable antibody domain. Like a whole antibody, it is able to bind selectively to a specific antigen. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805, which are incorporated, by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

The term “antigen” as provided herein refers to molecules capable of binding to the antibody binding domain provided herein. An “antigen binding domain” as provided herein is a region of an antibody that binds to an antigen (epitope). As described above, the antigen binding domain may include one constant and one variable domain of each of the heavy and the light chain (VL, VH, CL and CH1, respectively). In embodiments, the antigen binding domain includes a light chain variable domain and a heavy chain variable domain. In embodiments, the antigen binding domain includes light chain variable domain and does not include a heavy chain variable domain and/or a heavy chain constant domain. The paratope or antigen-binding site is formed on the N-terminus of the antigen binding domain. The two variable domains of an antigen binding domain may bind the epitope of an antigen.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Antibodies, e.g., recombinant, monoclonal, or polyclonal antibodies, can be prepared by many techniques known in the art (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. In embodiments, the antibodies described herein include humanized and/or chimeric monoclonal antibodies.

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

For specific proteins (antibodies or fragments thereof) described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the protein is the protein as identified by its NCBI sequence reference. In embodiments, the protein is the protein as identified by its NCBI sequence reference, homolog or functional fragment thereof.

The term “CD4” as referred to herein is a glycoprotein expressed on the surface of T helper cells, regulatory T cells, monocytes, macrophages, and dendritic cells. CD4 was originally known as leu-3 and T4 (after the OKT4 monoclonal antibody). CD4 as referred to herein has four immunoglobulin domains (D₁ to D₄) that are exposed on the extracellular surface of the cell, see ENTREZ No. 920, UNIPROT No. P01730, and GENBANK® Accession No. NP_000607, which are incorporated by reference.

The term “CD8” as referred to herein is a transmembrane glycoprotein that serves as a co-receptor for the T cell receptor (TCR). Like the TCR, CD8 binds to a major histocompatibility complex (MHC) molecule, but is specific for the class I MHC protein, see ENTREZ No. 925 and UNIPROT No. P01732, which are incorporated by reference herein.

The term “CD19 protein” or “CD19” as used herein includes any of the recombinant or naturally-occurring forms of B-lymphocyte antigen CD19, also known as CD19 molecule (Cluster of Differentiation 19), B-Lymphocyte Surface Antigen B4, T-Cell Surface Antigen Leu-12 and CVID3, or variants or homologs thereof that maintain CD19 activity (e.g. within at least 50%, 80%, 90%, 95%, 96%, 97%, 98%, 99% or 100% activity compared to CD19). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD19 protein. In embodiments, the CD19 protein is substantially identical to the protein identified by the UniProt reference number P15391 or a variant or homolog having substantial identity thereto.

The term “CD20 protein” or “CD20” as used herein includes any of the recombinant or naturally-occurring forms of B-lymphocyte antigen CD20, also known as CD20 molecule (Cluster of Differentiation 20), or variants or homologs thereof that maintain CD20 activity (e.g. within at least 50%, 80%, 90%, 95%, 96%, 97%, 98%, 99% or 100% activity compared to CD20). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD20 protein. In embodiments, the CD20 protein is substantially identical to the protein identified by the UniProt reference number P11836 or a variant or homolog having substantial identity thereto.

The term “CD27 protein” or “CD27” as used herein includes any of the recombinant or naturally-occurring forms of T-Cell Activation Antigen CD27, also known as CD27 molecule (Cluster of Differentiation 27), Tumor Necrosis Factor Receptor Superfamily Member 7, T Cell Activation Antigen 5152 or variants or homologs thereof that maintain CD27 activity (e.g. within at least 50%, 80%, 90%, 95%, 96%, 97%, 98%, 99% or 100% activity compared to CD27). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD27 protein. In embodiments, the CD27 protein is substantially identical to the protein identified by the UniProt reference number P26842 or a variant or homolog having substantial identity thereto.

The term “CD38 protein” or “CD38” as used herein includes any of the recombinant or naturally-occurring forms of CD38 (Cluster of Differentiation 38), also known as cyclic ADP ribose hydrolase, 2′-phospho-ADP-ribosyl cyclase, or variants or homologs thereof that maintain CD38 activity (e.g. within at least 50%, 80%, 90%, 95%, 96%, 97%, 98%, 99% or 100% activity compared to CD38). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD38 protein. In embodiments, the CD38 protein is substantially identical to the protein identified by the UniProt reference number P28907 or a variant or homolog having substantial identity thereto.

A “CD73 protein” or “CD73 antigen” as referred to herein includes any of the recombinant or naturally-occurring forms of the Cluster of Differentiation 73 (CD73) also known as 5′-nucleotidase (5′-NT) or ecto-5′-nucleotidase or variants or homologs thereof that maintain CD73 nucleotidase activity (e.g. within at least 50%, 80%, 90%, 95%, 96%, 97%, 98%, 99% or 100% activity compared to CD73). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD73 protein. In embodiments, the CD73 protein is substantially identical to the protein identified by the UniProt reference number 21589 or a variant or homolog having substantial identity thereto. In embodiments, the CD73 protein is substantially identical to the protein identified by the UniProt reference number Q61503 or a variant or homolog having substantial identity thereto.

“Oleclumab” or “MEDI9447” refers to the anti-CD73 antibody described by Statement on a Nonproprietary Name Adopted by the USAN Council (May 26, 2016), and Hay et al. Oncoimmunology, 5(8) (2016).

“AD2” as provided herein refers the anti-CD73 antibody described by Borrione P et al., “CD38 stimulation lowers the activation threshold and enhances the alloreactivity of cord blood T cells by activating the phosphatidylinositol 3-kinase pathway and inducing CD73 expression.” J Immunol 162:6238-46 (1999).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch (e.g., bind), wherein the two species may be, for example, an antibody construct as described herein and a cancer protein. In embodiments, contacting includes, for example, allowing an antibody construct to bind to a cancer protein expressed on a cancer cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “B cell” or “B lymphocyte” provided herein are used interchangeably and refer to their standard meaning known in the biological arts. B cells are a type of white blood cell (leukocyte) capable of developing into antibody-producing cells, also referred to herein as “mature B cell” or “differentiated B cell.” The terms a “differentiated B cell” or “mature B cell” includes antibody-secreting B cells (e.g., plasmablasts, plasma cells) as well as memory B cells, which present antibodies on their cell surface. Differentiated B cells reside in secondary lymphoid organs and are characterized by the specific Immunoglobulin (Ig) they express. In embodiments, the differentiated B cell expresses IgM, IgG, IgA, IgE or a combination thereof. In embodiments, the differentiated B cell expresses IgM. In embodiments, the differentiated B cell expresses IgG. In embodiments, the differentiated B cell expresses IgA. In embodiments, the differentiated B cell expresses IgE.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As defined herein, the term “activation”, “activate”, “activating”, “activator” and the like in reference to a cell (e.g., B cell)-ligand interaction means positively affecting (e.g. increasing) the activity or function of the cell relative to the activity or function of the cell in the absence of the ligand. In aspects activation means positively affecting (e.g. increasing) the proliferation rate or biologic activity of the cell relative to the rate or activity of the cell in the absence of the activator. The terms may reference activation, or activating, sensitizing, or up-regulating signal transduction or enzymatic activity or gene expression of a cell. Thus, activation may include, at least in part, partially or totally increasing stimulation, increasing or enabling activation, or activating, sensitizing, or up-regulating signal transduction or enzymatic activity or gene expression relative to the absence of the activator. Activation may include, at least in part, partially or totally increasing stimulation, increasing or enabling activation, or activating, sensitizing, or up-regulating signal transduction or enzymatic activity or gene expression.

The terms “agonist,” “activator,” “upregulator,” etc. refer to a substance capable of detectably increasing the activity or proliferation of a given cell. The agonist can increase activity or proliferation by 10%, 20%, 30%, 40%, 50%, 60%, 70%, 80%, 90% or more in comparison to a control in the absence of the agonist. In embodiments, proliferation or activity is 1.5-fold, 2-fold, 3-fold, 4-fold, 5-fold, 10-fold or higher than the proliferation or activity in the absence of the agonist.

The term “modulator” refers to an agent that increases or decreases the level of a target molecule or the function of a target molecule or the physical state of the target of the molecule relative to the absence of the modulator. In embodiments, the modulator increases or decreases the proliferation rate of a cell (e.g., B cell) or the function of a cell or the physical state of a cell relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. cancer, or infectious disease) means that the disease (e.g. cancer, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's disease, and non-Hodgkin's lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound (antibody or other agent) described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. In embodiments, administering refers to subcutaneous administration. In embodiments, administering refers to intravenous administration, including infusion or bolus. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

As used herein “treating cancer” and “treating a cancer tumor” means preventing an increase in size or volume of the cancer tumor. In embodiments, the cancer tumor is a solid tumor. In embodiments, treating a cancer tumor includes decreasing the size of volume of a cancer tumor. In embodiments, treating a cancer tumor includes eliminating the cancer tumor altogether. In embodiments, a cancer tumor is eliminated when it is not detectable by an imaging test such as magnetic resonance imaging (MRI), a positron emission tomography (PET) scan, X-ray computed tomography (CT), ultrasound, or single-photon emission computed tomography (SPECT). In embodiments, treating a cancer tumor further comprises reducing or preventing metastasis of the cancer tumor.

### Anti-CD73 Antibodies

The anti-CD73 antibodies that may be used for the methods or are included in the compositions provided herein including embodiments thereof are, inter alia, capable of binding CD73 proteins and inhibiting CD73 catalytic activity. Any of the anti-CD73 antibodies (e.g., 1E9 antibodies) described in WO 2017/100670 can be used for the methods and compositions provided herein. Any antibody capable of binding the same epitope as antibody 1E9 may be used for the methods and compositions provided herein including embodiments thereof. The antibodies provided herein are capable of binding a CD73 protein, activate and redistribute B cells and include the CDRs (CDR L1, CDR L2, CDR L3, CDR H1, CDR H2, and CDR H3) or functional fragments thereof of the mouse monoclonal antibody 1E9.

The terms “mouse monoclonal antibody 1E9,” “monoclonal antibody 1E9,” “antibody 1E9,” “1E9 antibody,” and “1E9” refer to the 1E9 antibody described by Thomson et al, Tissue Antigens 2008, Volume 35, Issue 1: Production and characterization of monoclonal antibodies to the glycosyl phosphatidylinositol-anchored lymphocyte differentiation antigen ecto-5′-nucleotidase (CD73). The antibodies provided herein including embodiments thereof may include one or more CDR of a 1E9 antibody. In embodiments, the antibody includes a 1E9 antibody CDR. A “1E9 antibody CDR” and “1E9 CDR” refer to a CDR of the 1E9 antibody having an antibody light chain of SEQ ID NO:11 and an antibody heavy chain of SEQ ID NO:12.

“CPI-006” as provided herein refers to a humanized CD73 antibody comprising an 1E9 CDR L1 having SEQ ID NO:1, an 1E9 CDR L2 having SEQ ID NO:2, an 1E9 CDR L3 having SEQ ID NO:3, an 1E9 CDR H1 having SEQ ID NO:4, an 1E9 CDR H2 having SEQ ID NO:5, and an 1E9 CDR H3 having SEQ ID NO:6. In embodiments, CPI-006 refers to a humanized CD73 antibody comprising a light chain variable region of SEQ ID NO:8 and a heavy chain variable region of SEQ ID NO:7. In embodiments, CPI-006 refers to a humanized CD73 antibody comprising a light chain of SEQ ID NO:10 and a heavy chain of SEQ ID NO:9. CPI-006 and embodiments thereof are also described in WO 2017/100670, which is incorporated by reference herein in its entirety.

In embodiments, the anti-CD73 antibody provided herein comprises a humanized light chain variable region including an 1E9 CDR L1, an 1E9 CDR L2, and an 1E9 CDR L3 and a humanized heavy chain variable region including an 1E9 CDR H1, an 1E9 CDR H2, and an 1E9 CDR H3. In embodiments, the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and the CDR H3 has a sequence of SEQ ID NO:6. In embodiments, the humanized light chain variable region comprises at least one binding framework region residue. In embodiments, the humanized heavy chain variable region comprises at least one binding framework region residue. A framework region residue involved in (or important for) epitope binding (e.g. CD73 binding) is referred to herein as a binding framework region residue. The binding framework region residues resides in the framework region of a humanized light chain variable region (i.e. FR L1, FR L2, FR L3, FR L4) or they may reside in the framework of a humanized heavy chain variable region (i.e. FR H1, FR H2, FR H3, FR H4). A binding framework residue residing in the FR L3 region of a humanized light chain is referred to herein as a FR L3 binding framework region residue. Thus, a binding framework region residue residing in the FR H3 region of a humanized heavy chain is referred to herein as a FR H3 binding framework region residue.

In embodiments, the anti-CD73 antibody provided herein comprises a humanized light chain variable region and a humanized heavy chain variable region. The humanized light chain variable region comprises: (i) a CDR L1 as set forth in SEQ ID NO:1, a CDR L2 as set forth in SEQ ID NO:2, a CDR L3 as set forth in SEQ ID NO:3 and (ii) a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87.

In embodiments, the humanized heavy chain variable region comprises: (i) a mouse CDR H1 as set forth in SEQ ID NO:4, a mouse CDR H2 as set forth in SEQ ID NO:5, and a mouse CDR H3 as set forth in SEQ ID NO:6 and (ii) an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

In embodiments, the humanized light chain variable region comprises a binding framework region residue that is a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87.

In embodiments, the humanized light chain variable region comprises a binding framework region residue that is a valine at a position corresponding to Kabat position 2. In embodiments, the humanized light chain variable region comprises a binding framework region residue that is a methionine at a position corresponding to Kabat position 4. In embodiments, the humanized light chain variable region comprises a binding framework region residue that is an aspartic acid or a leucine at a position corresponding to Kabat position 9. In embodiments, the humanized light chain variable region comprises a binding framework region residue that is a proline or a serine at a position corresponding to Kabat position 12. In embodiments, the humanized light chain variable region comprises a binding framework region residue that is a lysine or a proline at a position corresponding to Kabat position 18. In embodiments, the humanized light chain variable region comprises a binding framework region residue that is a alanine at a position corresponding to Kabat position 43. In embodiments, the humanized light chain variable region comprises a binding framework region residue that is a proline or a serine at a position corresponding to Kabat position 60.

In embodiments, the humanized light chain variable region comprises a binding framework region residue that is a threonine at a position corresponding to Kabat position 74. In embodiments, the humanized light chain variable region comprises a binding framework region residue that is an asparagine or a serine at a position corresponding to Kabat position 76. In embodiments, the humanized light chain variable region comprises a binding framework region residue that is an asparagine or a serine at a position corresponding to Kabat position 77. In embodiments, the humanized light chain variable region comprises a binding framework region residue that is an isoleucine or a leucine at a position corresponding to Kabat position 78. In embodiments, the humanized light chain variable region comprises a binding framework region residue that is a serine or an alanine at a position corresponding to Kabat position 80. In embodiments, the humanized light chain variable region comprises a binding framework region residue that is a glutamine at a position corresponding to Kabat position 100. In embodiments, the humanized light chain variable region comprises a binding framework region residue that is a valine at a position corresponding to Kabat position 104. In embodiments, the humanized light chain variable region comprises a binding framework region residue that is a glutamic acid or an alanine at a position corresponding to Kabat position 1. In embodiments, the humanized light chain variable region comprises a binding framework region residue that is a glutamine at a position corresponding to Kabat position 3.

In embodiments, the humanized light chain variable region comprises a binding framework region residue that is a phenylalanine or a threonine at a position corresponding to Kabat position 10. In embodiments, the humanized light chain variable region comprises a binding framework region residue that is a glutamine at a position corresponding to Kabat position 11. In embodiments, the humanized light chain variable region comprises a binding framework region residue that is an alanine or a leucine at a position corresponding to Kabat position 13. In embodiments, the humanized light chain variable region comprises a binding framework region residue that is a threonine at a position corresponding to Kabat position 14. In embodiments, the humanized light chain variable region comprises a binding framework region residue that is a valine or a proline at a position corresponding to Kabat position 15. In embodiments, the humanized light chain variable region comprises a binding framework region residue that is a lysine at a position corresponding to Kabat position 16. In embodiments, the humanized light chain variable region comprises a binding framework region residue that is a glutamic acid or an aspartic acid at a position corresponding to Kabat position 17. In embodiments, the humanized light chain variable region comprises a binding framework region residue that is a threonine at a position corresponding to Kabat position 22.

In embodiments, the humanized light chain variable region comprises a binding framework region residue that is a lysine at a position corresponding to Kabat position 42. In embodiments, the humanized light chain variable region comprises a binding framework region residue that is an arginine at a position corresponding to Kabat position 45. In embodiments, the humanized light chain variable region comprises a binding framework region residue that is an isoleucine at a position corresponding to Kabat position 58. In embodiments, the humanized light chain variable region comprises a binding framework region residue that is a tyrosine at a position corresponding to Kabat position 67. In embodiments, the humanized light chain variable region comprises a binding framework region residue that is a phenylalanine at a position corresponding to Kabat position 73. In embodiments, the humanized light chain variable region comprises a binding framework region residue that is an isoleucine at a position corresponding to Kabat position 78. In embodiments, the humanized light chain variable region comprises a binding framework region residue that is a tyrosine at a position corresponding to Kabat position 85. In embodiments, the humanized light chain variable region comprises a binding framework region residue that is a phenylalanine at a position corresponding to Kabat position 87.

In embodiments, the humanized heavy chain variable region provided herein comprises a binding framework region residue that is an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

In embodiments, the humanized heavy chain variable region comprises a binding framework region residue that is an isoleucine at a position corresponding to Kabat position 37. In embodiments, the humanized heavy chain variable region comprises a binding framework region residue that is an alanine or a proline at a position corresponding to Kabat position 40. In embodiments, the humanized heavy chain variable region comprises a binding framework region residue that is a lysine at a position corresponding to Kabat position 43. In embodiments, the humanized heavy chain variable region comprises a binding framework region residue that is a serine at a position corresponding to Kabat position 70. In embodiments, the humanized heavy chain variable region comprises a binding framework region residue that is an isoleucine or a threonine at a position corresponding to Kabat position 75. In embodiments, the humanized heavy chain variable region comprises a binding framework region residue that is a tryptophan at a position corresponding to Kabat position 82. In embodiments, the humanized heavy chain variable region comprises a binding framework region residue that is an arginine or a lysine at a position corresponding to Kabat position 83. In embodiments, the humanized heavy chain variable region comprises a binding framework region residue that is a alanine at a position corresponding to Kabat position 84.

In embodiments, the humanized heavy chain variable region comprises a binding framework region residue that is a serine at a position corresponding to Kabat position 85. In embodiments, the humanized heavy chain variable region comprises a binding framework region residue that is a valine or a methionine at a position corresponding to Kabat position 89. In embodiments, the humanized heavy chain variable region comprises a binding framework region residue that is a valine at a position corresponding to Kabat position 5. In embodiments, the humanized heavy chain variable region comprises a binding framework region residue that is a serine at a position corresponding to Kabat position 7. In embodiments, the humanized heavy chain variable region comprises a binding framework region residue that is a valine at a position corresponding to Kabat position 11. In embodiments, the humanized heavy chain variable region comprises a binding framework region residue that is a glutamic acid or a lysine at a position corresponding to Kabat position 12. In embodiments, the humanized heavy chain variable region comprises a binding framework region residue that is an isoleucine or a valine at a position corresponding to Kabat position 20. In embodiments, the humanized heavy chain variable region comprises a binding framework region residue that is an arginine at a position corresponding to Kabat position 38. In embodiments, the humanized heavy chain variable region comprises a binding framework region residue that is an arginine at a position corresponding to Kabat position 66. In embodiments, the humanized heavy chain variable region comprises a binding framework region residue that is an valine at a position corresponding to Kabat position 67.

In embodiments, the humanized heavy chain variable region comprises a binding framework region residue that is an isoleucine at a position corresponding to Kabat position 69. In embodiments, the humanized heavy chain variable region comprises a binding framework region residue that is an alanine at a position corresponding to Kabat position 71. In embodiments, the humanized heavy chain variable region comprises a binding framework region residue that is a lysine at a position corresponding to Kabat position 73. In embodiments, the humanized heavy chain variable region comprises a binding framework region residue that is a threonine at a position corresponding to Kabat position 87. In embodiments, the humanized heavy chain variable region comprises a binding framework region residue that is a glutamic acid at a position corresponding to Kabat position 1. In embodiments, the humanized heavy chain variable region comprises a binding framework region residue that is a valine at a position corresponding to Kabat position 24. In embodiments, the humanized heavy chain variable region comprises a binding framework region residue that is a arginine at a position corresponding to Kabat position 44. In embodiments, the humanized heavy chain variable region comprises a binding framework region residue that is a methionine at a position corresponding to Kabat position 48. In embodiments, the humanized heavy chain variable region comprises a binding framework region residue that is a leucine at a position corresponding to Kabat position 80. In embodiments, the humanized heavy chain variable region comprises a binding framework region residue that is a glutamic acid at a position corresponding to Kabat position 81.

In embodiments, the humanized light chain variable region comprises a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18; and the humanized heavy chain variable region comprises an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

In embodiments, the humanized light chain variable region comprises a valine at a position corresponding to Kabat position 2, a methionine at a position corresponding to Kabat position 4, a leucine at a position corresponding to Kabat position 9, a proline at a position corresponding to Kabat position 12, and a proline at a position corresponding to Kabat position 18; and the humanized heavy chain variable region comprises an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

In embodiments, the humanized light chain variable region comprises a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18; and the humanized heavy chain variable region comprises an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, and a methionine at a position corresponding to Kabat position 89.

In embodiments, the humanized light chain variable region comprises a valine at a position corresponding to Kabat position 2, a methionine at a position corresponding to Kabat position 4, a leucine at a position corresponding to Kabat position 9, a proline at a position corresponding to Kabat position 12, and a proline at a position corresponding to Kabat position 18; and the humanized heavy chain variable region comprises an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, and a methionine at a position corresponding to Kabat position 89.

In embodiments, the humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and the humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and the humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and the humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, the humanized light chain variable region comprises a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and the humanized heavy chain variable region comprises a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, humanized light chain variable region comprises a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87; and the humanized heavy chain variable region comprises a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

In embodiments, humanized light chain variable region comprises a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87; and the humanized heavy chain variable region comprises a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

In embodiments, humanized light chain variable region comprises a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87; and the humanized heavy chain variable region comprises a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

In embodiments, humanized light chain variable region comprises a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87; and the humanized heavy chain variable region comprises a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

In embodiments, the humanized heavy chain variable region comprises s a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

In embodiments, the humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

In embodiments, the humanized heavy chain variable region comprises the sequence of SEQ ID NO:7. In embodiments, the humanized heavy chain variable region is SEQ ID NO:7. In embodiments, the humanized light chain variable region comprises the sequence of SEQ ID NO:8. In embodiments, the humanized light chain variable region is SEQ ID NO:8. In embodiments, the humanized heavy chain variable region comprises the sequence of SEQ ID NO:7, and the humanized light chain variable region comprises the sequence of SEQ ID NO:8. In embodiments, the humanized heavy chain variable region is SEQ ID NO:7, and the humanized light chain variable region is SEQ ID NO:8.

In embodiments, the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and the CDR H3 has a sequence of SEQ ID NO:6. In embodiments, the humanized heavy chain variable region includes the sequence of SEQ ID NO:7. In embodiments, the humanized light chain variable region includes the sequence of SEQ ID NO:8. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv).

In embodiments, the anti-CD73 antibody is oleclumab, BMS-986179, IPH5301, or AD2. In embodiments, the anti-CD73 antibody is oleclumab. In embodiments, the anti-CD73 antibody is BMS-986179. In embodiments, the anti-CD73 antibody is IPH5301. In embodiments, the anti-CD73 antibody is AD2.

The anti-CD73 antibodies as provided herein may be Fab′ fragments. Where the anti-CD73 antibodies are Fab′ fragments, the anti-CD73 antibodies include a humanized heavy chain (e.g. including a constant and a variable region) and a humanized light chain (e.g. including a constant and a variable region). In embodiments, the anti-CD73 antibody is a Fab′ fragment. In embodiments, the anti-CD73 antibody is a F(ab′)₂ fragment. In embodiments, the anti-CD73 antibody comprises a human constant region. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is an IgA. In embodiments, the anti-CD73 antibody is an IgM.

In embodiments, the anti-CD73 antibody is a single chain antibody. A single chain antibody comprises a variable light chain and a variable heavy chain. A person of skill in the art will immediately recognize that a single chain antibody comprises a single light chain and a single heavy chain, in contrast to an immunoglobulin antibody, which comprises two identical pairs of polypeptide chains, each pair having one light chain and one heavy chain. Each light chain and heavy chain in turn consists of two regions: a variable (“V”) region (i.e. variable light chain and variable heavy chain) involved in binding the target antigen, and a constant (“C”) region that interacts with other components of the immune system. The variable light chain and the variable heavy chain in a single chain antibody may be linked through a linker peptide. Examples for linker peptides of single chain antibodies are described in Bird, R. E., Hardman, K. D., Jacobson, J. W., Johnson, S., Kaufman, B. M., Lee, S. M., Lee, T., Pope, S. H., Riordan, G. S. and Whitlow, M. (1988). Methods of making scFv antibodies have been described. See, Huse et al., Science 246:1275-1281 (1989); Ward et al., Nature 341:544-546 (1989); and Vaughan et al., Nature Biotech. 14:309-314 (1996). Briefly, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell.

The ability of an antibody to bind a specific epitope (e.g., CD73) can be described by the equilibrium dissociation constant (K_{D}). The equilibrium dissociation constant (K_{D}) as defined herein is the ratio of the dissociation rate (K-off) and the association rate (K-on) of an anti-CD73 antibody to a CD73 protein. It is described by the following formula: K_{D}=K-off/K-on. In embodiments, the anti-CD73 antibody is capable of binding a CD73 antigen with an equilibrium dissociation constant (K_{D}) from about 0.5 to about 25 nM. In embodiments, the anti-CD73 antibody is capable of binding a CD73 antigen with an equilibrium dissociation constant (K_{D}) from about 1 to about 25 nM. In embodiments, the anti-CD73 antibody is capable of binding a CD73 antigen with an equilibrium dissociation constant (K_{D}) from about 1.5 to about 25 nM. In embodiments, the anti-CD73 antibody is capable of binding a CD73 antigen with an equilibrium dissociation constant (K_{D}) from about 2 to about 25 nM. In embodiments, the anti-CD73 antibody is capable of binding a CD73 antigen with an equilibrium dissociation constant (K_{D}) from about 2.5 to about 25 nM. In embodiments, the anti-CD73 antibody is capable of binding a CD73 antigen with an equilibrium dissociation constant (K_{D}) from about 3 to about 25 nM. In embodiments, the anti-CD73 antibody is capable of binding a CD73 antigen with an equilibrium dissociation constant (K_{D}) from about 3.5 to about 25 nM. In embodiments, the anti-CD73 antibody is capable of binding a CD73 antigen with an equilibrium dissociation constant (K_{D}) from about 4 to about 25 nM. In embodiments, the anti-CD73 antibody is capable of binding a CD73 antigen with an equilibrium dissociation constant (K_{D}) in this paragraph at a pH below 7.5. In embodiments, the anti-CD73 antibody is capable of binding a CD73 antigen with an equilibrium dissociation constant (K_{D}) in this paragraph at a pH of less than about 7.5. In embodiments, the anti-CD73 antibody is capable of binding a CD73 antigen with an equilibrium dissociation constant (K_{D}) in this paragraph at a pH of less than about 7.0. In embodiments, the anti-CD73 antibody is capable of binding a CD73 antigen with an equilibrium dissociation constant (K_{D}) in this paragraph at a pH of less than about 6.5. In embodiments, the anti-CD73 antibody is capable of binding a CD73 antigen with an equilibrium dissociation constant (K_{D}) in this paragraph at a pH of less than about 6.0. In embodiments, the anti-CD73 antibody is capable of binding a CD73 antigen with an equilibrium dissociation constant (K_{D}) in this paragraph at a pH of less than about 5.5. In embodiments, the anti-CD73 antibody is capable of binding a CD73 antigen with an equilibrium dissociation constant (K_{D}) in this paragraph at a pH of less than about 5. In embodiments, the anti-CD73 antibody is capable of binding a CD73 antigen with an equilibrium dissociation constant (K_{D}) in this paragraph at a pH of less than about 4.5. In embodiments, the anti-CD73 antibody is capable of binding a CD73 antigen with an equilibrium dissociation constant (K_{D}) in this paragraph at a pH from about 6.0 to about 7.0. In embodiments, the anti-CD73 antibody is capable of binding a CD73 antigen with an equilibrium dissociation constant (K_{D}) in this paragraph at a pH of about 6.0. In embodiments, the anti-CD73 antibody is capable of binding a CD73 antigen with an equilibrium dissociation constant (K_{D}) in this paragraph at a pH of about 6.1. In embodiments, the anti-CD73 antibody is capable of binding a CD73 antigen with an equilibrium dissociation constant (K_{D}) in this paragraph at a pH of about 6.2. In embodiments, the anti-CD73 antibody is capable of binding a CD73 antigen with an equilibrium dissociation constant (K_{D}) in this paragraph at a pH of about 6.3. In embodiments, the anti-CD73 antibody is capable of binding a CD73 antigen with an equilibrium dissociation constant (K_{D}) in this paragraph at a pH of about 6.4. In embodiments, the anti-CD73 antibody is capable of binding a CD73 antigen with an equilibrium dissociation constant (K_{D}) in this paragraph at a pH of about 6.5. In embodiments, the anti-CD73 antibody is capable of binding a CD73 antigen with an equilibrium dissociation constant (K_{D}) in this paragraph at a pH of about 6.6. In embodiments, the anti-CD73 antibody is capable of binding a CD73 antigen with an equilibrium dissociation constant (K_{D}) in this paragraph at a pH of about 6.7. In embodiments, the anti-CD73 antibody is capable of binding a CD73 antigen with an equilibrium dissociation constant (K_{D}) in this paragraph at a pH of about 6.8. In embodiments, the anti-CD73 antibody is capable of binding a CD73 antigen with an equilibrium dissociation constant (K_{D}) in this paragraph at a pH of about 6.9. In embodiments, the anti-CD73 antibody is capable of binding a CD73 antigen with an equilibrium dissociation constant (K_{D}) in this paragraph at a pH of about 7.0.

The anti-CD73 antibody provided herein including embodiments and aspects thereof may include a glutamine at a position corresponding to Kabat position 297. In embodiments, the anti-CD73 antibody is bound to a CD73 antigen. In embodiments, the CD73 antigen forms part of a cell. In embodiments, the cell is a lymphoid cell. In embodiments, the cell is a B cell.

### Methods

The methods and compositions provided herein allow, inter alia, for the generation of antigen-specific antibodies in vivo in a subject. Applicants have surprisingly found that administration of an anti-CD73 antibody (e.g., CPI-006) to a subject (e.g., human cancer subject; human subject having COVID-19) induces differentiation of B cells and robust expansion of specific B cell clones in the subject. This B cell clonal expansion is consistent with antigen-specific B cell activation and generation of antibodies that are capable of binding specific antigens (e.g., cancer antigens, infectious disease antigens) expressed in the subject. The antibodies formed in vivo by the methods provided herein may be isolated from a subject, expressed and after their antigen-specificity has been determined, used for therapeutic or diagnostic purposes. For example, cancer antigen-binding antibodies produced in a cancer subject after the cancer subject has received an effective amount of the anti-CD73 antibody provided herein may be isolated, expressed and re-administered to the same cancer subject or another cancer subject. Likewise, the cancer antigen-binding antibodies produced in a cancer subject after the cancer subject has received an effective amount of the anti-CD73 antibody provided herein may be isolated, expressed and used for detection of a cancer antigen. Thus, the methods and compositions provided herein are, inter alia, useful for a variety of applications including personalized medicine.

The anti-CD73 antibodies used for the methods and included in the compositions provided herein including embodiments and aspects thereof are effective at treating infections, including viral infections, bacterial infections, fungal infections, and parasitic infections. Without intending to be bound by any particular theory or mechanism of action, the anti-CD73 antibodies described herein are effective at binding CD73 proteins; activating immune cells, including B cells and/or T cells; inducing an antibody response to a virus, bacteria, fungus, or parasite; and/or presenting antigens on B cells and macrophages.

The term “infection” or “infectious disease” refers to a disease or condition that is caused by organisms such as bacteria, viruses, fungi, or parasites. The terms “infection” and “infectious disease” refer to both a disease and to the strain of bacterium, virus, fungus, or parasite that cause that disease. Thus, treating an “infection” or “infectious disease” refers to: (i) treating the disease caused by the bacterium, virus, fungus, or parasite; and (ii) treating the bacterium, virus, fungus, or parasite, which necessarily results in treatment of the disease caused by the bacterium, virus, fungus, or parasite. For example, using the anti-CD73 antibodies described herein to treat SARS-CoV-2 or a SARS-CoV-2 infection also necessarily refers to treating COVID-19 because SARS-CoV-2 is the cause of COVID-19. Thus, methods of treating a bacterium, virus, fungus, or parasite also refers to treating the disease caused by the bacterium, virus, fungus, or parasite even if the disease is not explicitly recited in the disclosure because the skilled artisan would know and understand what diseases are caused by the bacteria, viruses, fungi, and parasites described herein.

The disclosure provides methods of treating an infection or infectious disease in a subject in need thereof by administering to the subject an effective amount of an anti-CD73 antibody described herein. In embodiments, the anti-CD73 antibody is CPI-006. In embodiments, the methods further comprise administering a second therapeutic drug to treat the infectious disease. In embodiments, the methods further comprise administering an antigenic agent (e.g., peptide) that is an infectious disease antigenic agent or an infectious disease peptide. In embodiments, the infection or infectious disease is hand foot and mouth disease, acute flaccid myelitis, anaplasmosis, anthrax, California serogroup virus disease, chikungunya virus disease, Eastern equine encephalitis virus disease, powassan virus disease, St. Louis encephalitis virus disease, Japanese encephalitis (e.g., epidemic encephalitis B), West Nile virus disease, Western equine encephalitis virus disease, babesiosis, botulism, brucellosis, Campylobacteriosis, a Campylobacter infection (e.g., a Campylobacter jejuni infection), a Candida infection (e.g., a Candida auris infection), a carbapenemase producing carbapenem-resistant enterobacteriaceae (CP-CRE) infection (e.g., a CP-CRE Enterobacter spp. infection, a Escherichia coli infection, a Klebsiella spp. infection), chancroid, a Chlamydia infection (e.g., a Chlamydia trachomatis infection), cholera, a Clostridium difficile infection, a Clostridium perfringens infection, coccidiodomycosis, syphilis, cryptosporidiosis, cyclosporiasis, dysentery, infectious diarrhea, Dengue virus infection, diphtheria, an Anaplasma phagocytophilum infection, an Ehrlichia chaffeensis infection, an Ehrlichia ewingii infection, giardiasis, gonorrhea, Haemophilus influenzae, H1N1 influenza, H5N1 influenza, H7N9 influenza, human avian influenza, Hansen's disease (i.e., leprosy), a hantavirus infection, hantavirus hemorrhagic fever (e.g., hantavirus hemorrhagic fever with renal syndrome), non-hantavirus pulmonary syndrome, hemolytic uremic syndrome, hepatitis A, hepatitis B, hepatitis C, hepatitis E, HIV infection, influenza-associated pediatric mortality, pneumococcal disease, legionellosis, leptospirosis, listeriosis, Lyme disease, malaria (e.g., P. vivax malaria), measles, meningococcal disease, meningococcal meningitis, mumps, influenza A virus infection, pertussis, bubonic plague, septicemic plague, pneumonic plague, polio, psittacosis, Q fever, rabies, rubella, Salmonella (e.g., Salmonella paratyphi infection, Salmonella typhi infection), salmonellosis, scarlet fever, severe acute syndrome-associated coronavirus disease (e.g., SARS-Co-V, SARS-CoV-1, SAR-CoV-2, MERS, COVID-19), shiga toxin-producing Escherichia coli, shigellosis, smallpox, spotted fever rickettsiosis, streptococcal toxic shock syndrome, tetanus, toxic shock syndrome, trichinellosis, tuberculosis, tularemia, methicillin-resistant Staphylococcus aureus (MRSA), vancomycin-resistant Staphylococcus aureus, varicella, vibriosis, viral hemorrhagic fever, epidemic hemorrhagic fever, Crimean-Congo hemorrhagic fever virus, Ebola virus, Lassa virus, Lujo virus, Marburg virus, Guanarito virus, Junin virus, Machupo virus, Sabia virus, yellow fever, Zika virus infection, Creutzfeldt-Jacob disease (e.g., transmissible spongiform encephalopathy), herpes simplex-1, herpes simplex-2, genital herpes, herpes zoster (e.g., shingles), or human papillomavirus. In embodiments, the infectious disease is human avian influenza. In embodiments, the infectious disease is herpes. In embodiments, the infectious disease is herpes simplex-1. In embodiments, the infectious disease is genital herpes. In embodiments, the infectious disease is herpes zoster. In embodiments, the infectious disease is human papillomavirus. In embodiments, the infectious disease is hand foot and mouth disease. In embodiments, the infectious disease is hantavirus hemorrhagic fever. In embodiments, the infectious disease is epidemic hemorrhagic fever. In embodiments, the infectious disease is hantavirus hemorrhagic fever with renal syndrome. In embodiments, the infectious disease is dysentery. In embodiments, the infectious disease is acute flaccid myelitis. In embodiments, the infectious disease is anaplasmosis. In embodiments, the infectious disease is Clostridium difficile infection. In embodiments, the infectious disease is Clostridium perfringens. In embodiments, the infectious disease is scarlet fever. In embodiments, the infectious disease is Creutzfeldt-Jacob disease (transmissible spongiform encephalopathy). In embodiments, the infectious disease is anthrax. In embodiments, the infectious disease is an arboviral disease. In embodiments, the infectious disease is California serogroup virus disease. In embodiments, the infectious disease is chikungunya virus disease. In embodiments, the infectious disease is Eastern equine encephalitis virus disease. In embodiments, the infectious disease is powassan virus disease. In embodiments, the infectious disease is St. Louis encephalitis virus disease. In embodiments, the infectious disease is West Nile virus disease. In embodiments, the infectious disease is Western equine encephalitis virus disease. In embodiments, the infectious disease is Japanese encephalitis. In embodiments, the infectious disease is babesiosis. In embodiments, the infectious disease is botulism. In embodiments, the infectious disease is brucellosis. In embodiments, the infectious disease is Campylobacteriosis. In embodiments, the infectious disease is Candida auris. In embodiments, the infectious disease is carbapenemase producing carbapenem-resistant enterobacteriaceae (CP-CRE). In embodiments, the infectious disease is CP-CRE Enterobacter spp. In embodiments, the infectious disease is Escherichia coli. In embodiments, the infectious disease is Klebsiella spp. In embodiments, the infectious disease is chancroid. In embodiments, the infectious disease is Chlamydia trachomatis infection. In embodiments, the infectious disease is cholera. In embodiments, the infectious disease is infectious diarrhea. In embodiments, the infectious disease is coccidiodomycosis. In embodiments, the infectious disease is syphilis. In embodiments, the infectious disease is COVID-19. In embodiments, the infectious disease is cryptosporidiosis. In embodiments, the infectious disease is cyclosporiasis. In embodiments, the infectious disease is Dengue virus infection. In embodiments, the infectious disease is diphtheria. In embodiments, the infectious disease is Anaplasma phagocytophilum infection. In embodiments, the infectious disease is Ehrlichia chaffeensis infection. In embodiments, the infectious disease is Ehrlichia ewingii infection. In embodiments, the infectious disease is giardiasis. In embodiments, the infectious disease is gonorrhea. In embodiments, the infectious disease is Haemophilus influenzae. In embodiments, the infectious disease is Hansen's disease. In embodiments, the infectious disease is hantavirus infection. In embodiments, the infectious disease is non-hantavirus pulmonary syndrome. In embodiments, the infectious disease is hemolytic uremic syndrome. In embodiments, the infectious disease is hepatitis A. In embodiments, the infectious disease is hepatitis B. In embodiments, the infectious disease is hepatitis C. In embodiments, the infectious disease is HIV infection. In embodiments, the infectious disease is influenza-associated pediatric mortality. In embodiments, the infectious disease is pneumococcal disease. In embodiments, the infectious disease is legionellosis. In embodiments, the infectious disease is leptospirosis. In embodiments, the infectious disease is listeriosis. In embodiments, the infectious disease is Lyme disease. In embodiments, the infectious disease is malaria. In embodiments, the infectious disease is P. vivax malaria. In embodiments, the infectious disease is measles. In embodiments, the infectious disease is meningococcal disease. In embodiments, the infectious disease is meningococcal meningitis. In embodiments, the infectious disease is mumps. In embodiments, the infectious disease is influenza A virus infection. In embodiments, the infectious disease is pertussis. In embodiments, the infectious disease is Bubonic plague. In embodiments, the infectious disease is septicemic plague. In embodiments, the infectious disease is pneumonic plague. In embodiments, the infectious disease is H1N1 influenza. In embodiments, the infectious disease is H5N1 influenza. In embodiments, the infectious disease is H7N9 influenza. In embodiments, the infectious disease is polio. In embodiments, the infectious disease is psittacosis. In embodiments, the infectious disease is Q fever. In embodiments, the infectious disease is rabies. In embodiments, the infectious disease is rubella. In embodiments, the infectious disease is Salmonella. In embodiments, the infectious disease is Salmonella paratyphi infection. In embodiments, the infectious disease is Salmonella typhi infection. In embodiments, the infectious disease is salmonellosis. In embodiments, the infectious disease is severe acute syndrome-associated coronavirus disease. In embodiments, the infectious disease is shiga toxin-producing Escherichia coli. In embodiments, the infectious disease is shigellosis. In embodiments, the infectious disease is smallpox. In embodiments, the infectious disease is spotted fever rickettsiosis. In embodiments, the infectious disease is streptococcal toxic shock syndrome. In embodiments, the infectious disease is tetanus. In embodiments, the infectious disease is toxic shock syndrome. In embodiments, the infectious disease is trichinellosis. In embodiments, the infectious disease is tuberculosis. In embodiments, the infectious disease is tularemia. In embodiments, the infectious disease is methicillin-resistant Staphylococcus aureus (MRSA). In embodiments, the infectious disease is vancomycin-resistant Staphylococcus aureus. In embodiments, the infectious disease is varicella. In embodiments, the infectious disease is vibriosis. In embodiments, the infectious disease is viral hemorrhagic fever. In embodiments, the infectious disease is Crimean-Congo hemorrhagic fever virus. In embodiments, the infectious disease is Ebola virus. In embodiments, the infectious disease is Lassa virus. In embodiments, the infectious disease is Lujo virus. In embodiments, the infectious disease is Marburg virus. In embodiments, the infectious disease is Guanarito virus. In embodiments, the infectious disease is Junin virus. In embodiments, the infectious disease is Machupo virus. In embodiments, the infectious disease is Sabia virus. In embodiments, the infectious disease is yellow fever. In embodiments, the infectious disease is Zika virus infection. In embodiments, the infectious disease is bacterial mycetoma.

The disclosure provides methods of treating a bacterial infection or bacterial disease in a subject in need thereof by administering to the subject an effective amount of an anti-CD73 antibody described herein. In embodiments, the anti-CD73 antibody is CPI-006. In embodiments, the methods further comprise administering an effective amount of an antibiotic to the subject. In embodiments, the methods further comprise administering an antigenic agent (e.g., peptide) that is an bacterial antigenic agent or a bacterial antigenic peptide. In embodiments, the bacterial infection is an antibiotic-resistant bacterial infection. In embodiments, the bacterial infection or bacterial disease is a Haemophilus infection (e.g., H. aphrophilus, H. aegyptius, H. ducreyi, H. infuenzae, H. infuenzae type a, H. infuenzae type b, H. infuenzae type c, H. infuenzae type d, H. infuenzae type e, H. infuenzae type f, H. haemolyticus, H. parainfuenzae, H. parahaemolyticus), anAcinetobacter infection (e.g., antibiotic-resistant Acinetobacter, carbapenem-resistantAcinetobacter), a Clostridioides difficile infection (e.g., antibiotic-resistant Clostridioides difficile, Enterobacteriaceae (e.g., antibiotic-resistant Enterobacteriaceae), Neisseria gonorrhoeae (e.g., antibiotic-resistant Neisseria gonorrhoeae), Campylobacter (e.g., antibiotic-resistant Campylobacter), Extended-Spectrum β-Lactamase (ESBL)-producing an Enterobacteriaceae infection (e.g, antibiotic-resistant ESBL-producing Enterobacteriaceae), an Enterococcus infection (e.g., antibiotic-resistant Enterococcus, vancomycin-resistant Enterococcus), a Pseudomonas infection (e.g., Pseudomonas aeruginosa, antibiotic-resistant Pseudomonas aeruginosa, multidrug-resistant Pseudomonas aeruginosa, Pseudomonas mallei, antibiotic-resistant Pseudomonas mallei, multidrug-resistant Pseudomonas mallei, Pseudomonas pseudomallei, antibiotic-resistant Pseudomonas pseudomallei, multidrug-resistant Pseudomonas pseudomallei), a Salmonella infection (e.g., antibiotic-resistant Salmonella, nontyphoidal Salmonella, antibiotic-resistant nontyphoidal Salmonella), typhoid (e.g., antibiotic-resistant typhoid), paratyphoid, a Shigella infection (e.g., antibiotic-resistant Shigella), a Staphylococcus infection (e.g., S. aureus, antibiotic-resistant S. aureus, methicillin-resistant S. aureus (MRSA), S. epidermidis, S. saprophiticus), a Streptococcus infection (e.g., S. pneumoniae, antibiotic-resistant S. pneumoniae, S. pyogenes, S. agalactiae, S. dysgalactiae, S. gallotyticus, S. anginosus, S. sanguinis, S. suis, S. mitis, S. mutans), tuberculosis (e.g., antibiotic-resistant tuberculosis, multidrug-resistant tuberculosis), a Group A streptococcus infection (e.g., antibiotic-resistant Group A streptococcus, erythromycin-resistant Group A streptococcus), a Group B streptococcus infection (e.g., antibiotic-resistant Group B streptococcus, clindamycin-resistant Group B streptococcus), aMycoplasma genitalium infection (e.g., antibiotic-resistant Mycoplasma genitalium), a Bordetella pertussis infection (e.g., antibiotic-resistant Bordetella pertussis), aMycoplasma pneumoniae infection (e.g., antibiotic-resistantMycoplasma pneumoniae), nocardiosis, a Nocardia infection (e.g., N. brasiliensis, N. cyriacigeorgica, N. farcinica, N. nova, N. asteroides, N. caviae), Helicobacter pylori, meningitis (e.g., caused by Neisseria meningitidis), meningococcal meningitis, bubonic plague (e.g., caused by Yersinia pestis), Legionnaires' disease (e.g., caused by legionella bacteria), a Mycobacterium infection (e.g., atypical Mycobacterium infection, M. leprae, M tuberculosis, M. tuberculosis complex, M. avium, M. avium complex, M. avium paratuberculosis, M. bovis, M. africanum, M. microti, M. canetti, M. chelonae, M. lepromatosis, M. marinum, M. kansasii, M. abscessus, M ulcerans, M. scrofulaceum, M. fortuitum), Hansen's disease (e.g., a Mycobacterium leprae infection), Q fever (e.g., a Coxiella burnetii infection), rheumatic fever (e.g., a Group A streptococcus infection), or anthrax. In embodiments, the bacterial infection is a Haemophilus infection. In embodiments, the bacterial infection is a H. aphrophilus infection. In embodiments, the bacterial infection is a H. aegyptius infection. In embodiments, the bacterial infection is a H. ducreyi infection. In embodiments, the bacterial infection is a H. infuenzae infection. In embodiments, the bacterial infection is a H. infuenzae type a infection. In embodiments, the bacterial infection is a H. infuenzae type b infection. In embodiments, the bacterial infection is a H. infuenzae type c infection. In embodiments, the bacterial infection is a H. infuenzae type d infection. In embodiments, the bacterial infection is a H. infuenzae type e infection. In embodiments, the bacterial infection is a H. infuenzae type f infection. In embodiments, the bacterial infection is a H. haemolyticus infection. In embodiments, the bacterial infection is a H. parainfuenzae infection. In embodiments, the bacterial infection is a H. parahaemolyticus infection. In embodiments, the bacterial infection is Acinetobacter. In embodiments, the bacterial infection is an antibiotic-resistantAcinetobacter. In embodiments, the bacterial infection is a carbapenem-resistantAcinetobacter. In embodiments, the bacterial infection is Clostridioides difficile. In embodiments, the bacterial infection is an antibiotic-resistant Clostridioides difficile. In embodiments, the bacterial infection is Enterobacteriaceae. Enterobacteriaceae is a large, heterogeneous group of gram-negative bacteria that include Escherichia, Citrobacter, Enterobacter, Proteus, Hafnia, Klebsiella, Providencia, Serratia, Morganella, Providencia, Cronobacter, and Edwardsiella. In embodiments, the bacterial infection is Morganella. In embodiments, the bacterial infection is Providencia. In embodiments, the bacterial infection is Edwardsiella. In embodiments, the bacterial infection is Cronobacter. In embodiments, the bacterial infection is Escherichia, In embodiments, the bacterial infection is Citrobacter. In embodiments, the bacterial infection is Enterobacter. In embodiments, the bacterial infection is Proteus. In embodiments, the bacterial infection is Hafnia. In embodiments, the bacterial infection is Klebsiella. In embodiments, the bacterial infection is Providencia. In embodiments, the bacterial infection is Serratia. In embodiments, the bacterial infection is antibiotic-resistant Enterobacteriaceae. In embodiments, the bacterial infection is Neisseria gonorrhoeae. In embodiments, the bacterial disease is gonorrhoeae. In embodiments, the bacterial infection is antibiotic-resistant Neisseria gonorrhoeae. In embodiments, the bacterial infection is Campylobacter. In embodiments, the bacterial infection is antibiotic-resistant Campylobacter. In embodiments, the bacterial infection is extended-spectrum β-lactamase (ESBL)-producing Enterobacteriaceae. In embodiments, the bacterial infection is antibiotic-resistant ESBL-producing Enterobacteriaceae. In embodiments, the bacterial infection is Enterococcus. In embodiments, the bacterial infection is antibiotic-resistant Enterococcus. In embodiments, the bacterial infection is vancomycin-resistant Enterococcus. In embodiments, the bacterial infection is a Pseudomonas bacterial infection. In embodiments, the bacterial infection is Pseudomonas aeruginosa. In embodiments, the bacterial infection is antibiotic-resistant Pseudomonas aeruginosa. In embodiments, the bacterial infection is multidrug-resistant Pseudomonas aeruginosa. In embodiments, the bacterial infection is Pseudomonas mallei. In embodiments, the bacterial infection is antibiotic-resistant Pseudomonas mallei. In embodiments, the bacterial infection is Pseudomonas pseudomallei. In embodiments, the bacterial infection is antibiotic-resistant Pseudomonas pseudomallei. In embodiments, the bacterial infection is Mycobacterium infection. In embodiments, the bacterial infection is an atypical Mycobacterium infection. In embodiments, the bacterial infection is Mycobacterium leprae. In embodiments, the bacterial infection is Mycobacterium tuberculosis. In embodiments, the bacterial infection is Mycobacterium tuberculosis complex. In embodiments, the bacterial infection is Mycobacterium tuberculosis complex, which comprises M. tuberculosis, M. africanum, M. orygis, M. bovis, M. bovis bacillius calmette-Guerin, M. microti, M. canetti, M. caprae, M. pinnipedii, M. suricattae, M. mungi, or a combination of two or more thereof. In embodiments, the bacterial infection is Mycobacterium bovis. In embodiments, the bacterial infection is Mycobacterium africanum. In embodiments, the bacterial infection is Mycobacterium microti. In embodiments, the bacterial infection is Mycobacterium canetti. In embodiments, the bacterial infection is Mycobacterium chelonae. In embodiments, the bacterial infection is Mycobacterium avium. In embodiments, the bacterial infection is Mycobacterium avium avium. In embodiments, the bacterial infection is M. avium complex (MAC). In embodiments, the bacterial infection is Mycobacterium avium complex, which comprises M. avium, M. avium paratuberculosis, M. avium silvaticum, M. avium hominissuis, M. colombiense, M. indicus pranii, M. intracellulare, or a combination of two or more thereof. In embodiments, the bacterial infection is Mycobacterium avium paratuberculosis (e.g., Johne's disease, paratuberculosis, Crohn's disease, and inflammatory bowel disease). In embodiments, the bacterial infection is Mycobacterium lepromatosis (e.g., leprosy). In embodiments, the bacterial infection is Mycobacterium marinum (e.g., aquarium granuloma). In embodiments, the bacterial infection is Mycobacterium kansasii. In embodiments, the bacterial infection is Mycobacterium abscessus. In embodiments, the bacterial infection is Mycobacterium ulcerans (e.g., Buruli ulcer). In embodiments, the bacterial infection is Mycobacterium scrofulaceum. In embodiments, the bacterial infection is Mycobacterium fortuitum. In embodiments, the bacterial infection is Salmonella. In embodiments, the bacterial infection is antibiotic-resistant Salmonella. In embodiments, the bacterial infection is nontyphoidal Salmonella. In embodiments, the bacterial infection is antibiotic-resistant nontyphoidal Salmonella. In embodiments, the bacterial infection is typhoid. In embodiments, the bacterial disease is paratyphoid. In embodiments, the bacterial infection is antibiotic-resistant typhoid. In embodiments, the bacterial infection is Shigella. In embodiments, the bacterial infection is antibiotic-resistant Shigella. In embodiments, the bacterial infection is Staphylococcus bacterial infection. In embodiments, the bacterial infection is Staphylococcus aureus. In embodiments, the bacterial infection is antibiotic-resistant Staphylococcus aureus. In embodiments, the bacterial infection is methicillin-resistant Staphylococcus aureus. In embodiments, the bacterial infection is methicillin-susceptible Staphylococcus aureus. In embodiments, the bacterial infection is Staphylococcus epidermidis. In embodiments, the bacterial infection is Staphylococcus saprophiticus. In embodiments, the bacterial infection is a Streptococcus bacterial infection. In embodiments, the bacterial infection is Streptococcus pneumoniae. In embodiments, the bacterial infection is antibiotic-resistant Streptococcus pneumoniae. In embodiments, the bacterial infection is Streptococcus pyogenes. In embodiments, the bacterial infection is Streptococcus agalactiae. In embodiments, the bacterial infection is Streptococcus dysgalactiae. In embodiments, the bacterial infection is Streptococcus gallotyticus. In embodiments, the bacterial infection is Streptococcus anginosus. In embodiments, the bacterial infection is Streptococcus sanguinis. In embodiments, the bacterial infection is Streptococcus suis. In embodiments, the bacterial infection is Streptococcus mitis. In embodiments, the bacterial infection is Streptococcus mutans. In embodiments, the bacterial disease is tuberculosis. In embodiments, the bacterial disease is antibiotic-resistant tuberculosis. In embodiments, the bacterial disease is multidrug-resistant tuberculosis. In embodiments, the bacterial infection is Group A streptococcus. In embodiments, the bacterial infection is antibiotic-resistant Group A streptococcus. In embodiments, the bacterial infection is erythromycin-resistant Group A streptococcus. In embodiments, the bacterial infection is Group B streptococcus. In embodiments, the bacterial infection is antibiotic-resistant Group B streptococcus. In embodiments, the bacterial infection is clindamycin-resistant Group B streptococcus. In embodiments, the bacterial infection is Mycoplasma genitalium. In embodiments, the bacterial infection is antibiotic-resistant Mycoplasma genitalium. In embodiments, the bacterial infection is Bordetella pertussis. In embodiments, the bacterial infection is antibiotic-resistant Bordetella pertussis. In embodiments, the bacterial infection is Mycoplasma pneumoniae. In embodiments, the bacterial infection is antibiotic-resistant Mycoplasma pneumoniae. In embodiments, the bacterial infection is Nocardia. In embodiments, the bacterial infection is nocardiosis. In embodiments, the bacterial infection is N. brasiliensis. In embodiments, the bacterial infection is N. cyriacigeorgica. In embodiments, the bacterial infection is N. farcinica. In embodiments, the bacterial infection is N. nova. In embodiments, the bacterial infection is N. asteroides. In embodiments, the bacterial infection is N. caviae. In embodiments, the bacterial infection is Helicobacter pylori. In embodiments, the bacterial infection is meningitis. In embodiments, the bacterial infection is Neisseria meningitidis. In embodiments, the bacterial infection is bubonic plague. In embodiments, the bacterial infection is epidemic cerebrospinal meningitis. In embodiments, the bacterial infection is meningococcal meningitis. In embodiments, the bacterial disease is bubonic plague. In embodiments, the bacterial disease is septicemic plague. In embodiments, the bacterial disease is pneumonic plague. In embodiments, the bacterial disease is Legionnaires' disease. In embodiments, the bacterial infection is anthrax. In embodiments, the bacterial disease is Hansen's disease. In embodiments, the bacterial disease is Q fever. In embodiments, the bacterial disease is rheumatic fever.

The disclosure provides methods of treating a fungal infection or fungal disease in a subject in need thereof by administering to the subject an effective amount of an anti-CD73 antibody described herein. In embodiments, the anti-CD73 antibody is CPI-006. In embodiments, the methods further comprise administering to the subject an antifungal agent. In embodiments, the methods further comprise administering an antigenic agent (e.g., peptide) that is a fungal disease antigenic agent or fungal disease peptide. In embodiments, the fungal disease is a drug-resistant fungal disease. In embodiments, the fungal disease is a multidrug-resistant fungal disease. In embodiments, the fungal disease is an antibiotic-resistant fungal disease. In embodiments, the fungal disease is aspergillosis (e.g., including antifungal resistant aspergillosis), blastomycosis, candidiasis, a Candida infection (e.g., Candida auris, antifungal-resistant Candida auris, Candida albicans, anti fungal-resistant Candida albicans, Candida glabrata, antifungal-resistant Candida glabrata, Candida parapsilosis, antifungal-resistant Candida parapsilosis), valley fever (caused by the fungus Coccidioides), a Cryptococcus neoformans infection, a Cryptococcus gattii infection, a fungal eye infection (e.g., a Fusarium infection, a Aspergillosis infection, a Candida infection), a fungal nail infection, fungal meningitis (e.g., a Cryptococcus infection, a Histoplasma infection, a Blastomyces infection, a Coccidioides infection, or a Candida infection), histoplasmosis, mucormycosis, fungal mycetoma, Pneumocystis pneumonia, ringworm, sporotrichosis (e.g., cutaneous, pulmonary, or disseminated), paracoccidioidomycosis, or talaromycosis. In embodiments, the fungal disease is aspergillosis. In embodiments, the fungal disease is antifungal resistant aspergillosis. In embodiments, the fungal disease is blastomycosis. In embodiments, the fungal disease is candidiasis. In embodiments, the fungal disease is a Candida fungal infection. In embodiments, the fungal disease is a Candida auris infection. In embodiments, the fungal infection is antifungal resistant Candida auris. In embodiments, the fungal infection is Candida albicans. In embodiments, the fungal infection is antifungal resistant Candida albicans. In embodiments, the fungal infection is Candida glabrata. In embodiments, the fungal infection is antifungal resistant Candida glabrata. In embodiments, the fungal infection is Candida parapsilosis. In embodiments, the fungal infection is antifungal resistant Candida parapsilosis. In embodiments, the fungal disease is valley fever. In embodiments, the fungal infection is a Coccidioides infection. In embodiments, the fungal infection is a Cryptococcus neoformans fungal infection. In embodiments, the fungal infection is a Cryptococcus gattii fungal infection. In embodiments, the fungal disease is a fungal eye infection. In embodiments, the fungal disease is an eye infection caused by Fusarium. In embodiments, the fungal disease is an eye infection caused by Aspergillosis. In embodiments, the fungal disease is an eye infection caused by Candida. In embodiments, the fungal infection is a fungal nail infection. In embodiments, the fungal disease is fungal meningitis. In embodiments, the fungal disease is fungal meningitis caused by Cryptococcus. In embodiments, the fungal disease is fungal meningitis caused by Histoplasma. In embodiments, the fungal disease is fungal meningitis caused by Blastomyces. In embodiments, the fungal disease is fungal meningitis caused by Coccidioides. In embodiments, the fungal disease is fungal meningitis caused by Candida. In embodiments, the fungal disease is histoplasmosis. In embodiments, the fungal disease is mucormycosis. In embodiments, the fungal disease is fungal mycetoma. In embodiments, the fungal disease is fungal mycetoma caused by eumycetoma. In embodiments, the fungal disease is Pneumocystis pneumonia. In embodiments, the fungal disease is ringworm. In embodiments, the fungal disease is sporotrichosis. In embodiments, the fungal disease is cutaneous sporotrichosis. In embodiments, the fungal disease is pulmonary sporotrichosis. In embodiments, the fungal disease is disseminated sporotrichosis. In embodiments, the fungal disease is paracoccidioidomycosis. In embodiments, the fungal disease is talaromycosis.

The disclosure provides methods of treating a parasitic disease or parasitic infection in a subject in need thereof by administering to the subject an effective amount of an anti-CD73 antibody described herein. In embodiments, the anti-CD73 antibody is CPI-006. In embodiments, the methods further comprise administering an effective amount of an antiparasite drug to the subject. In embodiments, the methods further comprise administering an antigenic agent (e.g., peptide) that is a parasitic disease antigenic agent or a parasitic disease peptide. In embodiments, the parasitic disease or infection is schistosomiasis, kala-azar, paragonimiasis (e.g., lung fluke, flatworm), an Acanthamoeba infection, an Acanthamoeba keratitis infection, African sleeping sickness, alveolar echinococcosis, amebiasis, Chagas disease (also known as American Trypanosomiasis), hookworm, ancylostomiasis, angiostrongyliasis, ansiakiasis, ascarisasis, babesiosis, balantidiasis, balamuthia, baylisascariasis, bilharzia, Blastocystis hominis infection, pediculosis (body lice, pubic lice, head lice), capillariasis, cercarial dermatitis, Chilomastix mesnili infection, clonorchiasis, cryptosporidiosis, cyclosporiasis, cysticercosis, Cystoisospora infection, Dientamoeba fragilis infection, diphyllobothriasis, Dipylidium caninum, tapeworms (e.g., Taenia infection), Hydatid disease (e.g., caused by Echinococcus granulosus), dirofilariasis, guinea worm disease, echinococcosis, elephantiasis, Endolimax nana infection, Entaboeba coli infection, Entamoeba dispar infection, Entamoeba hartmanni infection, Entamoeba histolytica infection, Entamoeba polecki infection, pinworms (e.g., Enterobiasis), fascioliasis, fasciolopsiasis, filariasis, giardiasis, gnathostomiasis, heterophyiasis, hydatid disease, hymenolepiasis, intestinal roundwords (e.g., Ascaris infection), Iodamoeba buetschii infection, keratitis, leishmaniasis, liver flukes (clonorchiasis), loiasis, malaria, microsporidiosis, scabies, myiasis, Naegleria infection, neurocysticercosis, ocular larva migrans, onchocerciasis (river blindness), opisthorchiasis, paragonimiasis, Pneumocystis jirovecii pneumonia, sappinia, sarcocystosis, toxoplasmosis, trichinosis, trichomoniasis, or whipworm infection. In embodiments, the parasitic disease or infection is schistosomiasis. In embodiments, the parasitic disease or infection is kala-azar. In embodiments, the parasitic disease or infection is paragonimiasis. In embodiments, the parasitic disease or infection is lung fluke. In embodiments, the parasitic disease or infection is flatworm. In embodiments, the parasitic disease or infection is an Acanthamoeba infection. In embodiments, the parasitic disease or infection is an Acanthamoeba keratitis infection. In embodiments, the parasitic disease or infection is African sleeping sickness. In embodiments, the parasitic disease or infection is alveolar echinococcosis. In embodiments, the parasitic disease or infection is amebiasis. In embodiments, the parasitic disease or infection is Chagas disease. In embodiments, the parasitic disease or infection is hookworm. In embodiments, the parasitic disease or infection is ancylostomiasis. In embodiments, the parasitic disease or infection is angiostrongyliasis. In embodiments, the parasitic disease or infection is ansiakiasis. In embodiments, the parasitic disease or infection is ascarisasis. In embodiments, the parasitic disease or infection is babesiosis. In embodiments, the parasitic disease or infection is balantidiasis. In embodiments, the parasitic disease or infection is balamuthia. In embodiments, the parasitic disease or infection is baylisascariasis. In embodiments, the parasitic disease or infection is bilharzia. In embodiments, the parasitic disease or infection is Blastocystis hominis infection. In embodiments, the parasitic disease or infection is pediculosis. In embodiments, the parasitic disease or infection is capillariasis. In embodiments, the parasitic disease or infection is cercarial dermatitis. In embodiments, the parasitic disease or infection is Chilomastix mesnili infection. In embodiments, the parasitic disease or infection is clonorchiasis. In embodiments, the parasitic disease or infection is cryptosporidiosis. In embodiments, the parasitic disease or infection is cyclosporiasis. In embodiments, the parasitic disease or infection is cysticercosis. In embodiments, the parasitic disease or infection is Cystoisospora infection. In embodiments, the parasitic disease or infection is Dientamoeba fragilis infection. In embodiments, the parasitic disease or infection is diphyllobothriasis. In embodiments, the parasitic disease or infection is Dipylidium caninum. In embodiments, the parasitic disease or infection is tapeworms. In embodiments, the parasitic disease or infection is hydatid disease. In embodiments, the parasitic disease or infection is dirofilariasis. In embodiments, the parasitic disease or infection is guinea worm disease. In embodiments, the parasitic disease or infection is echinococcosis. In embodiments, the parasitic disease or infection is elephantiasis. In embodiments, the parasitic disease or infection is Endolimax nana infection. In embodiments, the parasitic disease or infection is Entaboeba coli infection. In embodiments, the parasitic disease or infection is Entamoeba dispar infection. In embodiments, the parasitic disease or infection is Entamoeba hartmanni infection. In embodiments, the parasitic disease or infection is Entamoeba histolytica infection. In embodiments, the parasitic disease or infection is Entamoeba polecki infection. In embodiments, the parasitic disease or infection is pinworms. In embodiments, the parasitic disease or infection is fascioliasis. In embodiments, the parasitic disease or infection is fasciolopsiasis. In embodiments, the parasitic disease or infection is filariasis. In embodiments, the parasitic disease or infection is giardiasis. In embodiments, the parasitic disease or infection is gnathostomiasis. In embodiments, the parasitic disease or infection is heterophyiasis. In embodiments, the parasitic disease or infection is hydatid disease. In embodiments, the parasitic disease or infection is hymenolepiasis. In embodiments, the parasitic disease or infection is intestinal roundwords. In embodiments, the parasitic disease or infection is Iodamoeba buetschii infection. In embodiments, the parasitic disease or infection is keratitis. In embodiments, the parasitic disease or infection is leishmaniasis. In embodiments, the parasitic disease or infection is liver flukes. In embodiments, the parasitic disease or infection is loiasis. In embodiments, the parasitic disease or infection is malaria. In embodiments, the parasitic disease or infection is microsporidiosis. In embodiments, the parasitic disease or infection is scabies. In embodiments, the parasitic disease or infection is myiasis. In embodiments, the parasitic disease or infection is Naegleria infection. In embodiments, the parasitic disease or infection is neurocysticercosis. In embodiments, the parasitic disease or infection is ocular larva migrans. In embodiments, the parasitic disease or infection is onchocerciasis. In embodiments, the parasitic disease or infection is opisthorchiasis. In embodiments, the parasitic disease or infection is paragonimiasis. In embodiments, the parasitic disease or infection is Pneumocystis jirovecii pneumonia. In embodiments, the parasitic disease or infection is sappinia. In embodiments, the parasitic disease or infection is sarcocystosis. In embodiments, the parasitic disease or infection is toxoplasmosis. In embodiments, the parasitic disease or infection is trichinosis. In embodiments, the parasitic disease or infection is trichomoniasis. In embodiments, the parasitic disease or infection is whipworm infection.

The disclosure provides methods of treating a viral disease or viral infection in a subject in need thereof by administering to the subject an effective amount of an anti-CD73 antibody described herein. In embodiments, the anti-CD73 antibody is CPI-006. In embodiments, the methods further comprise administering an effective amount of an antiviral drug to the subject. The term “viral infection” refers to any infection caused by a virus. The viral infection can be any known in the art, such as a viral respiratory infection (e.g., COVID-19, MERS, bronchiolitis, common cold, croup, influenza, pneumonia); a viral gastrointestinal infection (e.g., norovirus, rotavirus, adenovirus, astrovirus); an exanthematous viral disease (e.g., measles, mumps, rubella, chickenpox, shingles, roseola, smallpox, fifth disease, chikungunya); a hepatic viral disease (e.g., hepatitis A, hepatitis B, hepatitis C, hepatitis D, hepatitis E); a cutaneous viral disease (e.g., warts, genital warts, herpes simplex, genital herpes, molluscum contagiosum); a viral hemorrhagic disease (e.g., Ebola, lassa fever, dengue fever, yellow fever, marburg hemorrhagic fever, Crimean-Congo hemorrhagic fever, Lujo virus, Marburg virus, Guanarito virus, Junin virus, Machupo virus, Sabia virus); a neurologic viral disease (e.g., polio, viral meningitis, viral encephalitis, rabies); Zika virus, West Nile virus, malaria, or tuberculosis. In embodiments, the viral infection is a viral respiratory infection; a viral gastrointestinal infection; an exanthematous viral disease; a hepatic viral disease; a cutaneous viral disease; a viral hemorrhagic disease; or a neurologic viral disease. In embodiments, the viral infection is SARS, bronchiolitis, common cold, croup, influenza, pneumonia, norovirus, rotavirus, adenovirus, astrovirus, measles, mumps, rubella, chickenpox, shingles, roseola, smallpox, fifth disease, chikungunya, hepatitis A, hepatitis B, hepatitis C, hepatitis D, hepatitis E, warts, genital warts, herpes simplex, HSV-1, HSV-2, genital herpes, molluscum contagiosum, Ebola, lassa fever, dengue fever, yellow fever, marburg hemorrhagic fever, hemorrhagic conjunctivitis, Crimean-Congo hemorrhagic fever, polio, viral meningitis, viral encephalitis, rabies, a Zika viral infection, a West Nile viral infection, Epstein Barr virus, or cytomegalovirus. In embodiments, the viral infection is mumps, cytomegalovirus, rubella, measles, or herpes simplex-1. In embodiments, the viral infection is viral respiratory infection. In embodiments, the viral infection is bronchiolitis. In embodiments, the viral infection is a common cold. In embodiments, the viral infection is croup. In embodiments, the viral infection is influenza. In embodiments, the viral infection is pneumonia, In embodiments, the viral infection is a viral gastrointestinal infection. In embodiments, the viral infection is norovirus. In embodiments, the viral infection is rotavirus. In embodiments, the viral infection is adenovirus. In embodiments, the viral infection is astrovirus. In embodiments, the viral infection is an exanthematous viral disease. In embodiments, the viral infection is measles. In embodiments, the viral infection is mumps. In embodiments, the viral infection is chicken pox. In embodiments, the viral infection is rubella. In embodiments, the viral infection is chickenpox. In embodiments, the viral infection is shingles. In embodiments, the viral infection is roseola. In embodiments, the viral infection is smallpox. In embodiments, the viral infection is fifth disease. In embodiments, the viral infection is chikungunya. In embodiments, the viral infection is a hepatic viral disease. In embodiments, the viral infection is, hepatitis A. In embodiments, the viral infection is hepatitis B. In embodiments, the viral infection is hepatitis C. In embodiments, the viral infection is hepatitis D. In embodiments, the viral infection is hepatitis E. In embodiments, the viral infection is a cutaneous viral disease. In embodiments, the viral infection is warts. In embodiments, the viral infection is genital warts. In embodiments, the viral infection is herpes simplex. In embodiments, the viral infection is HSV-1. In embodiments, the viral infection is HSV-2. In embodiments, the viral infection is genital herpes. In embodiments, the viral infection is molluscum contagiosum. In embodiments, the viral infection is a viral hemorrhagic disease. In embodiments, the viral infection is Ebola. In embodiments, the viral infection is lassa fever. In embodiments, the viral infection is dengue fever. In embodiments, the viral infection is yellow fever. In embodiments, the viral infection is marburg hemorrhagic fever. In embodiments, the viral infection is hemorrhagic conjunctivitis. In embodiments, the viral infection is Crimean-Congo hemorrhagic fever. In embodiments, the viral infection is a neurologic viral disease. In embodiments, the viral infection is polio. In embodiments, the viral infection is viral meningitis. In embodiments, the viral infection is viral encephalitis. In embodiments, the viral infection is rabies. In embodiments, the viral infection is a Zika viral infection. In embodiments, the viral infection is West Nile virus. In embodiments, the viral infection is Epstein Barr virus. In embodiments, the viral infection is cytomegalovirus. In embodiments, the methods further comprise administering an antiviral agent, an antiviral vaccine, or a combination thereof. In embodiments, the methods further comprise administering an antiviral agent. In embodiments, the methods further comprise administering an antiviral vaccine. In embodiments, the methods further comprise administering an antiviral agent and an antiviral vaccine. In embodiments, the methods further comprise administering an effective amount of a viral antigen. In embodiments, the methods further comprise administering an effective amount of antiviral agent, an antiviral vaccine, or a combination thereof. In embodiments, the methods further comprise administering an effective amount of a viral antigen.

The disclosure provides methods of treating a viral respiratory infection in a subject in need thereof by administering to the subject an effective amount of an anti-CD73 antibody described herein. In embodiments, the anti-CD73 antibody is CPI-006. In embodiments, the methods further comprise administering an effective amount of an antiviral drug to the subject. Viral respiratory infections include COVID-19, MERS, bronchiolitis, common cold, croup, influenza, and pneumonia. Viral respiratory infections can be caused by respiratory syncytial virus, rhinoviruses, cornonaviruses, parainfluenza viruses, influenza viruses, adenoviruses, enteroviruses, and human metapneumoviruses. In embodiments, the viral respiratory infection is a respiratory syncytial virus infection. In embodiments, the viral respiratory infection is an adenovirus infection. In embodiments, the viral respiratory infection is a parainfluenza virus infection. In embodiments, the viral respiratory infection is an influenza virus infection. In embodiments, the viral respiratory infection is a rhinovirus infection. In embodiments, the viral respiratory infection is an enterovirus infection. In embodiments, the viral respiratory infection is a human metapneumovirus infection. In embodiments, the viral respiratory infection is a coronavirus infection. In embodiments, the coronavirus is 229E (alpha coronavirus). In embodiments, the coronavirus is NL63 (alpha coronavirus). In embodiments, the coronoavirus is OC43 (beta coronavirus). In embodiments, the coronavirus is HKU1 (beta coronoavirus). In embodiments, the coronavirus is SARS-CoV. In embodiments, the coronavirus is SARS-CoV-1. In embodiments, the coronavirus is SARS-CoV-2. In embodiments, the coronavirus is MERS-CoV. In embodiments, the viral respiratory infection is COVID-19. In embodiments, the viral respiratory infection is MERS. “SARS” refers to severe acute respiratory syndrome. “SARS-CoV” refers to severe acute respiratory syndrome-associated coronavirus. “SARS-CoV-1” refers to severe acute respiratory syndrome-associated coronavirus 1. “SARS-CoV-2” refers to severe acute respiratory syndrome-associated coronavirus 2. “MERS-CoV” refers to Middle East respiratory syndrome-associated coronavirus. See, e.g., Chung et al, Genetic Characterization of Middle East Respiratory Syndrome Coronavirus, South Korea, 2018. Emerging Infectious Diseases, 25(5):958-962 (2019).

“COVID-19” refers to the disease caused by SARS-CoV-2. COVID-19 has an incubation period of 2-14 days, and symptoms include, e.g., fever, tiredness, cough, and shortness of breath (e.g., difficulty breathing). In embodiments, the disclosure provides methods of treating COVID-19 by administering to a patient an effective amount of the antibodies described herein, such as CPI-006.

“Middle East respiratory syndrome” or “MERS” refers to the disease caused by MERS-coronavirus. In embodiments, the disclosure provides methods of treating MERS by administering to a patient an effective amount of the antibodies described herein, such as CPI-006.

“Asymptomatic” means that a subject has a disease (e.g., as confirmed by positive PCR test results), but does not exhibit any symptoms of the disease. “Mild symptoms” or “mildly symptomatic” or “mild disease” in reference to SARS, SARS-CoV, SARS-CoV-1, SARS-CoV-2, and MERS-CoV refers to positive PCR test results, symptomatic, and 02 saturation of blood of at least 94% on room air. “Moderate symptoms” or “moderately symptomatic” or “moderate disease” in reference to SARS, SARS-CoV, SARS-CoV-1, SARS-CoV-2, and MERS-CoV refers to positive PCR test results, symptomatic, and β₂ saturation of blood of less than 94% on room air. “Severe symptoms” or “severely symptomatic” or “severe disease” in reference to SARS, SARS-CoV, SARS-CoV-1, SARS-CoV-2, and MERS-CoV refers to positive PCR test results, symptomatic, and the need for high flow O₂ therapy for respiratory support.

The term “viral antigen” refers to a viral substance that is capable of inducing an immune response in a subject. Viral antigens include a live virus, an inactivated virus, a toxoid produced by a virus, a viral vector, a viral subunit (e.g., protein or polysaccharide derived from the virus), or a nucleic acid (DNA or RNA). Viral antigens for specific viral diseases are well-known in the art. Strugnell, “Vaccine Antigens,” Understanding Modern Vaccines: Perspectives in Vaccinology, 1(1):61-88 (2011).

In embodiments, the disclosure provides methods of treating COVID-19 in a subject in need thereof by administering to a subject an effective amount of an anti-CD73 antibody described herein. In embodiments, the disclosure provides methods of treating COVID-19 in a subject in need thereof by administering to a subject an effective amount of CPI-006. In embodiments, the subject is asymptomic. In embodiments, the subject has mild symptoms. In embodiments, the subject has moderate symptoms. In embodiments, the subject has severe symptoms. In embodiments, the subject is hospitalized. In embodiments, the subject is hospitalized in an intensive care unit. In embodiments, the subject is on a ventilator. In embodiments, the subject is on a dialysis machine. In embodiments, the subject is on a ventilator and a dialysis machine.

In embodiments, the disclosure provides methods of treating Middle East respiratory syndrome (MERS) in a subject in need thereof by administering to a subject an effective amount of an anti-CD73 antibody described herein. In embodiments, the disclosure provides methods of treating MERS in a subject in need thereof by administering to a subject an effective amount of CPI-006. In embodiments, the subject is asymptomic. In embodiments, the subject has mild symptoms. In embodiments, the subject has moderate symptoms. In embodiments, the subject has severe symptoms. In embodiments, the subject is hospitalized. In embodiments, the subject is hospitalized in an intensive care unit. In embodiments, the subject is on a ventilator. In embodiments, the subject is on a dialysis machine. In embodiments, the subject is on a ventilator and a dialysis machine.

In embodiments, the disclosure provides methods of treating severe acute respiratory syndrome (SARS) in a subject in need thereof by administering an effective amount of an anti-CD73 antibody described herein. In embodiments, the disclosure provides methods of treating coronavirus in a subject in need thereof by administering an effective amount of an anti-CD73 antibody described herein. In embodiments, the anti-CD73 antibody is CPI-006. In embodiments, the coronavirus is 229E, NL63, OC43, or HKU1. In embodiments, the disclosure provides methods of treating severe acute respiratory syndrome coronavirus (SARS-CoV) in a subject in need thereof by administering an effective amount of an anti-CD73 antibody described herein. In embodiments, the disclosure provides methods of treating severe acute respiratory syndrome coronavirus 1 (SARS-CoV-1) in a subject in need thereof by administering an effective amount of an anti-CD73 antibody described herein. In embodiments, the disclosure provides methods of treating severe acute respiratory syndrome coronavirus 2 (SARS-CoV-2) in a subject in need thereof by administering an effective amount of an anti-CD73 antibody described herein. In embodiments, the disclosure provides methods of treating Middle East respiratory syndrome coronavirus (MERS-CoV) in a subject in need thereof by administering an effective amount of an anti-CD73 antibody described herein. In embodiments, the disclosure provides methods of treating MERS in a subject in need thereof by administering to a subject an effective amount of an anti-CD73 antibody described herein. In embodiments, the subject is asymptomic. In embodiments, the subject has mild symptoms. In embodiments, the subject has moderate symptoms. In embodiments, the subject has severe symptoms. In embodiments, the subject is hospitalized. In embodiments, the subject is hospitalized in an intensive care unit. In embodiments, the subject is on a ventilator. In embodiments, the subject is on a dialysis machine. In embodiments, the subject is on a ventilator and a dialysis machine.

In embodiments, the disclosure provides methods of improving humoral immune response in a subject having a viral infection in need thereof by administering an effective amount of an anti-CD73 antibody described herein. In embodiments, the anti-CD73 antibody is CPI-006. In embodiments, the disclosure provides methods of improving humoral immune response in a subject having a viral infection in need thereof by administering an effective amount of an anti-CD73 antibody described herein. In embodiments, the disclosure provides methods of improving humoral immune response in a subject having a viral respiratory infection in need thereof by administering an effective amount of an anti-CD73 antibody described herein. In embodiments, the anti-CD73 antibody is CPI-006. In embodiments, the viral respiratory infection is COVID-19. In embodiments, the viral respiratory infection is severe acute respiratory syndrome (SARS). In embodiments, the viral respiratory infection is coronavirus. In embodiments, the viral respiratory infection is SARS-coronavirus. In embodiments, the viral respiratory infection is SARS-coronavirus 1. In embodiments, the viral respiratory infection is SARS-coronavirus 2. In embodiments, the viral respiratory infection is MERS-coronavirus. In embodiments, the viral respiratory infection is bronchiolitis, common cold, croup, influenza, or pneumonia. In embodiments, the viral respiratory infection is caused by a respiratory syncytial virus, a rhinovirus, a coronavirus, a parainfluenza virus, an influenza virus, an adenovirus, an enterovirus, or a human metapneumovirus. In embodiments, the humoral immune response is improved compared to a control (e.g., a patient or group of patients who did not receive the anti-CD73 antibody described herein). In embodiments, the subject is asymptomatic, mildly symptomatic, moderately symptomatic, or severely symptomatic.

In embodiments, the disclosure provides methods of increasing serum or plasma immunoglobulin (IgM and/or IgG) anti-viral levels in a subject in need thereof by administering an effective amount of an anti-CD73 antibody described herein. In embodiments, the anti-CD73 antibody is CPI-006. In embodiments, the disclosure provides methods of increasing serum or plasma immunoglobulin (IgM and/or IgG) anti-SARS levels in a subject in need thereof by administering an effective amount of an anti-CD73 antibody described herein. In embodiments, the disclosure provides methods of increasing serum or plasma immunoglobulin (IgM and/or IgG) anti-SARS-CoV levels in a subject in need thereof by administering an effective amount of an anti-CD73 antibody described herein. In embodiments, the disclosure provides methods of increasing serum or plasma immunoglobulin (IgM and/or IgG) anti-coronavirus levels in a subject in need thereof by administering an effective amount of an anti-CD73 antibody described herein. In embodiments, the disclosure provides methods of increasing serum or plasma immunoglobulin (IgM and/or IgG) anti-SARS-CoV-1 levels in a subject in need thereof by administering an effective amount of an anti-CD73 antibody described herein. In embodiments, the disclosure provides methods of increasing serum or plasma immunoglobulin (IgM and/or IgG) anti-SARS-CoV-2 levels in a subject in need thereof by administering an effective amount of an anti-CD73 antibody described herein. In embodiments, the disclosure provides methods of increasing serum or plasma immunoglobulin (IgM and/or IgG) anti-MERS-CoV levels in a subject in need thereof by administering an effective amount of an anti-CD73 antibody described herein. In embodiments, the methods increase serum IgM levels in the subject. In embodiments, the methods increase serum IgG levels in the subject. In embodiments, the methods increase serum IgM levels and serum IgG levels in the subject. In embodiments, the methods increase plasma IgM levels in the subject. In embodiments, the methods increase plasma IgG levels in the subject. In embodiments, the methods increase plasma IgM levels and plasma IgG levels in the subject. In embodiments, the methods increase serum and plasma IgM levels in the subject. In embodiments, the methods increase serum and plasma IgG levels in the subject. In embodiments, the methods increase serum and plasma IgM levels and serum and plasma IgG levels in the subject. In embodiments, the subject is asymptomatic, mildly symptomatic, moderately symptomatic, or severely symptomatic. In embodiments, the increase in serum and/or plasma immunoglobulin (IgM and/or IgG) anti-SARS levels are an increase compared to a control (e.g., a patient or group of patients who did not receive the anti-CD73 antibody described herein).

In embodiments, the disclosure provides methods of increasing neutralizing antibodies to a virus in a subject in need thereof by administering an effective amount of an anti-CD73 antibody described herein. In embodiments, the anti-CD73 antibody is CPI-006. In embodiments, the disclosure provides methods of increasing neutralizing antibodies to a viral infection in a subject in need thereof by administering an effective amount of an anti-CD73 antibody described herein. In embodiments, the anti-CD73 antibody is CPI-006. In embodiments, the disclosure provides methods of increasing neutralizing antibodies to coronavirus in a subject in need thereof by administering an effective amount of an anti-CD73 antibody described herein. In embodiments, the anti-CD73 antibody is CPI-006. In embodiments, the disclosure provides methods of increasing neutralizing antibodies to SARS in a subject in need thereof by administering an effective amount of an anti-CD73 antibody described herein. In embodiments, the anti-CD73 antibody is CPI-006. In embodiments, the SARS is SARS-coronavirus. In embodiments, the SARS is SARS-coronavirus 1. In embodiments, the SARS is SARS-coronavirus 2. In embodiments, the SARS is MERS-coronavirus. In embodiments, the subject is asymptomatic, mildly symptomatic, moderately symptomatic, or severely symptomatic. In embodiments, the long-term immunity is compared to a control (e.g., a patient or group of patients who did not receive the anti-CD73 antibody described herein). The neutralizing antibodies prevent the virus from infecting target cells, e.g., by blocking the virus from binding to target cells

In embodiments, the disclosure provides methods of providing long-term immunity to a virus in a subject in need thereof by administering an effective amount of an anti-CD73 antibody described herein. In embodiments, the anti-CD73 antibody is CPI-006. In embodiments, the methods of providing long-term immunity to a virus is a method of preventing a reinfection of the virus in the subject. In embodiments, the anti-CD73 antibody is CPI-006. In embodiments, the methods of providing long-term immunity to a virus is a method of preventing new infection of the virus in the subject. In embodiments, the anti-CD73 antibody is CPI-006. In embodiments, the disclosure provides methods of providing long-term immunity to coronavirus in a subject in need thereof by administering an effective amount of an anti-CD73 antibody described herein. In embodiments, the anti-CD73 antibody is CPI-006. In embodiments, the disclosure provides methods of providing long-term immunity to SARS in a subject in need thereof by administering an effective amount of an anti-CD73 antibody described herein. In embodiments, the SARS is SARS-coronavirus. In embodiments, the SARS is SARS-coronavirus 1. In embodiments, the SARS is SARS-coronavirus 2. In embodiments, the SARS is MERS-coronavirus. In embodiments, the subject is asymptomatic, mildly symptomatic, moderately symptomatic, or severely symptomatic. In embodiments, the long-term immunity is compared to a control (e.g., a patient or group of patients who did not receive the anti-CD73 antibody described herein). In embodiments, long-term immunity is provided by increasing the antibody response to the virus. In embodiments, the long-term immunity is provided by increase cell-mediated immunity by T cells, which are also activated by anti-CD73 antibodies.

In embodiments, the disclosure provides decreasing disease severity, shortening the duration of illness, preventing complications, decreasing the duration and/or number of symptoms; decreasing the incidence of hospitalization; decreasing the length of hospitalization, decreasing the need for a medical procedures (e.g., intubation and/or dialysis) in a subject having a virus by administering an effective amount of an anti-CD73 antibody described herein. In embodiments, the anti-CD73 antibody is CPI-006. In embodiments, the disclosure provides decreasing the severity of SARS. In embodiments, the disclosure provides shortenint the duration of SARS. In embodiments, the disclosure provides decreasing the duration and/or number of symptoms related to SARS. In embodiments, the disclosure provides decreasing the incidence of hospitalization related to SARS. In embodiments, the disclosure provides decreasing the length of hospitalization related to SARS. In embodiments, the disclosure provides decreasing the need for a medical procedures (e.g., intubation and/or dialysis) related to SARS. In embodiments, the subject is asymptomatic, mildly symptomatic, moderately symptomatic, or severely symptomatic. In embodiments, the SARS is SARS-coronavirus. In embodiments, the SARS is SARS-coronavirus 1. In embodiments, the SARS is SARS-coronavirus 2. In embodiments, the SARS is MERS-coronavirus. In embodiments, the decrease is a decrease compared to a control (e.g., a patient or group of patients who did not receive the anti-CD73 antibody described herein).

In embodiments, the disclosure provides reducing the severity of COVID-19; decreasing the duration and/or number of symptoms related to COVID-19; decreasing the incidence of hospitalization related to COVID-19; decreasing the length of hospitalization related to COVID-19, decreasing the need for a medical procedures (e.g., intubation and/or dialysis) related to COVID-19 in a subject in need thereof by administering an effective amount of an anti-CD73 antibody described herein. In embodiments, the anti-CD73 antibody is CPI-006. In embodiments, the disclosure provides decreasing the duration and/or number of symptoms related to COVID-19. In embodiments, the disclosure provides decreasing the incidence of hospitalization related to COVID-19. In embodiments, the anti-CD73 antibody is CPI-006. In embodiments, the disclosure provides decreasing the length of hospitalization related to COVID-19. In embodiments, the disclosure provides decreasing the need for a medical procedures (e.g., intubation and/or dialysis) related to COVID-19. In embodiments, the anti-CD73 antibody is CPI-006. In embodiments, the subject is asymptomatic, mildly symptomatic, moderately symptomatic, or severely symptomatic. In embodiments, the subject is mildly symptomatic. In embodiments, the subject is moderately symptomatic. In embodiments, the decrease is a decrease compared to a control (e.g., a patient or group of patients who did not receive the anti-CD73 antibody described herein).

In an aspect, a method of immunostimulating a subject to treat a viral infection in a subject in need thereof by administering to the subject an effective amount of an anti-CD73 antibody described herein. In embodiments, the anti-CD73 antibody is CPI-006. In embodiments, the viral respiratory infection is COVID-19. In embodiments, the viral respiratory infection is SARS (e.g., SARS-CoV, SARS-CoV-1, SARS-CoV-2, MERS-CoV). In embodiments, the viral respiratory infection is bronchiolitis, common cold, croup, influenza, or penumonia. In embodiments, the viral respiratory infection is caused by respiratory syncytial virus, rhinoviruses, cornonaviruses, parainfluenza viruses, influenza viruses, adenoviruses, enteroviruses, or human metapneumoviruses. In embodiments, the viral respiratory infection is coronavirus (e.g., 229E, NL63, OC43, HKU1, SARS-CoV, SARS-CoV-1, SARS-CoV-2, MERS-CoV).

The term “immunostimulating” or “immunostimulation” as provided herein refers to the ability to activate the immune system or increasing activity of any of its components. In embodiments, the method of immunostimulating a subject comprises increasing inflammatory cytokines in the subject an effective amount of an anti-CD73 antibody. In embodiments, the anti-CD73 antibody is CPI-006. In embodiments, the method of immunostimulating a subject comprises increasing inflammatory cytokines in the subject, wherein the inflammatory cytokines are TNF-α, TNF-β, MIP-1α, MIP-10, IL-6, IL-10, IL-8, IP-10, MCP-1, MCP-2, IL-1Ra, GRO-α, MIP-3a, TNF-RII, IL-7, MMP-9, CRP, SAA, MMP-3, MDC, YKL-40, IL-27, or a combination of two or more thereof. In embodiments, the method of immunostimulating a subject comprises increasing inflammatory cytokines in the subject, wherein the inflammatory cytokines are TNF-α, TNF-β, MIP-1α, MIP-10, IL-6, IL-10, IL-8, IP-10, MCP-1, MCP-2, IL-1Ra, GRO-α, MIP-3a, TNF-RII, IL-7, MMP-9, or a combination of two or more thereof. In embodiments, the method of immunostimulating a subject comprises increasing inflammatory cytokines in the subject, wherein the inflammatory cytokines are TNF-α, TNF-β, MIP-1α, MIP-10, IL-6, IL-10, IL-8, IP-10, MCP-1, MCP-2, IL-1Ra, GRO-α, MIP-3a, or a combination of two or more thereof; and optionally wherein the inflammatory cytokines have a log₂-fold increase of at least two from about 0.5 hours to about 2 hours after administration of the anti-CD73 antibody. In embodiments, the method of immunostimulating a subject comprises increasing inflammatory cytokines in the subject, wherein the inflammatory cytokines are CRP, SAA, MMP-3, MDC, YKL-40, IL-27, or a combination of two or more thereof. In embodiments, the method of immunostimulating a subject comprises increasing inflammatory cytokines in the subject, wherein the inflammatory cytokines are C-reactive protein (CRP), serum amyloid A (SAA), or a combination thereof; and optionally wherein the inflammatory cytokines have a log₂ fold increase of at least two from about 1 day to about 8 days after administration of the anti-CD73 antibody. In embodiments, the method of immunostimulating a subject comprises increasing activation markers in the subject. In embodiments, the method of immunostimulating a subject comprises increasing antigen presenting cells in the subject. In embodiments, the method of immunostimulating a subject comprises increasing B cells in the subject. In embodiments, the method of immunostimulating a subject comprises increasing T cells in the subject. In embodiments, the method of immunostimulating a subject comprises increasing dendritic cells in the subject. In embodiments, the method of immunostimulating a subject comprises increasing antigen presenting cells in the subject, wherein the antigen-presenting cells express (i.e., comprise) CD3, CD14, CD19, CD25, CD69, CD83, CD86, MHC Class II (e.g., HLA-DR), BDCA-2, BDCA-4, CD11c^{low}, CD45RA, CD123, ILT-7, TLR7, TLR9, or a combination of two or more thereof. In embodiments, the method of immunostimulating a subject comprises decreasing monocytes in the subject, e.g., in the blood of the subject. In embodiments, the method of immunostimulating a subject comprises decreasing CD73^{NEG} CD8 T cells in the subject, e.g., in the blood of the subject. In embodiments, the method of immunostimulating a subject comprises decreasing CD73^{POS} CD8 T cells in the subject, e.g., in the blood of the subject. In embodiments, the method of immunostimulating a subject comprises increasing CD73^{NG} CD4 T cells in the subject, e.g., in the blood of the subject. In embodiments, the method of immunostimulating a subject comprises decreasing CD73^{POS} CD4 T cells in the subject, e.g., in the blood of the subject. In embodiments, the method of immunostimulating a subject comprises increasing the CD4/CD8 ratio in the subject, e.g., in the blood of the subject. In embodiments, the method of immunostimulating a subject comprises increasing the CD73^{NG}CD4/CD73^{NEG} CD8 ratio in the subject, e.g., in the blood of the subject.

In embodiments, the disclosure provides methods of preventing a viral infection in a subject in need thereof comprising administering to the subject an effective amount of a vaccine and an effective amount of an anti-CD73 antibody as described herein. In embodiments, the anti-CD73 antibody is CPI-006. In the methods of preventing a viral infection, the subject does not have the virus (e.g., as confirmed by PCR tests). In embodiments, the disclosure provides methods of preventing SARS in subject in need thereof comprising administering an effective amount of a SARS vaccine and an effective amount of an anti-CD73 antibody as described herein. In embodiments, the anti-CD73 antibody is CPI-006. In embodiments, the SARS is SARS-CoV, SARS-CoV-1, SARS-CoV-2, or MERS-CoV. In embodiments, the viral infection is COVID-19, MERS, bronchiolitis, common cold, croup, influenza, pneumonia, norovirus, rotavirus, adenovirus, astrovirus, measles, mumps, chicken pox, rubella, chickenpox, shingles, roseola, smallpox, fifth disease, chikungunya, hepatitis A, hepatitis B, hepatitis C, hepatitis D, hepatitis E, warts, genital warts, herpes simplex, genital herpes, molluscum contagiosum, Ebola, lassa fever, dengue fever, yellow fever, marburg hemorrhagic fever, Crimean-Congo hemorrhagic fever, polio, viral meningitis, viral encephalitis, rabies, Zika virus, West Nile virus, malaria, or tuberculosis.

In embodiments, the disclosure provides methods of treating a viral infection in an asymptomatic subject in need thereof comprising administering to the subject an effective amount of an anti-CD73 antibody as described herein. In embodiments, the anti-CD73 antibody is CPI-006. In embodiments, the disclosure provides methods of preventing SARS in an asymptomatic subject in need thereof comprising administering an effective amount of an anti-CD73 antibody as described herein. In embodiments, the anti-CD73 antibody is CPI-006. In embodiments, the SARS is SARS-CoV, SARS-CoV-1, SARS-CoV-2, or MERS-CoV. In embodiments, the methods prevent the onset of symptoms, reduce the duration of symptoms, or reduce the severity of symptoms. In embodiments, the viral infection is COVID-19, MERS, bronchiolitis, common cold, croup, influenza, pneumonia, norovirus, rotavirus, adenovirus, astrovirus, measles, mumps, chicken pox, rubella, chickenpox, shingles, roseola, smallpox, fifth disease, chikungunya, hepatitis A, hepatitis B, hepatitis C, hepatitis D, hepatitis E, warts, genital warts, herpes simplex, genital herpes, molluscum contagiosum, Ebola, lassa fever, dengue fever, yellow fever, marburg hemorrhagic fever, Crimean-Congo hemorrhagic fever, polio, viral meningitis, viral encephalitis, rabies, Zika virus, West Nile virus, malaria, or tuberculosis.

The disclosure provides methods of treating an infectious disease in a subject in need thereof by administering to the subject an effective amount of an anti-CD73 antibody described herein. In embodiments, the anti-CD73 antibody is CPI-006. In embodiments, the methods further comprise administering a second therapeutic drug to treat the infectious disease. In embodiments, the methods further comprise administering an antigenic agent (e.g., peptide) that is an infectious disease antigenic agent or an infectious disease peptide. In embodiments, the infectious disease is caused by a bacteria or pathogenic bacteria. Pathogenic bacteria are bacteria which cause diseases (e.g., in humans). In embodiments, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid, paratyphoid, diphtheria, syphilis, and leprosy. In embodiments, the disease is Bacterial vaginosis (i.e. bacteria that change the vaginal microbiota caused by an overgrowth of bacteria that crowd out the Lactobacilli species that maintain healthy vaginal microbial populations) (e.g., yeast infection, or Trichomonas vaginalis); Bacterial meningitis (i.e. a bacterial inflammation of the meninges); Bacterial pneumonia (i.e. a bacterial infection of the lungs); Urinary tract infection; Bacterial gastroenteritis; or Bacterial skin infections (e.g. impetigo, or cellulitis). In embodiments, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection. In embodiments, the infectious disease is caused by Epstein Barr virus. In embodiments, the infectious disease is caused by hepatitis C virus. In embodiments, the infectious disease is caused by hepatitis B virus. In embodiments, the infectious disease is caused by Ebola virus. In embodiments, the infectious disease is caused by herpes simplex virus. In embodiments, the infectious disease is caused by cytomegalovirus. In embodiments, the infectious disease is caused by chikungunya virus. In embodiments, the infectious disease is caused by dengue virus. In embodiments, the infectious disease is caused by plasmodium. In embodiments, the infectious disease is caused by mycobacterium. In embodiments, the infectious disease is caused by Epstein Barr virus, hepatitis C virus, hepatitis B virus, Ebola virus, herpes simplex virus, cytomegalovirus, chikungunya virus, dengue virus, plasmodium, or mycobacterium.

In embodiments, the disclosure provides methods of producing a cancer antigen-binding antibody by (i) administering to a cancer subject an effective amount of an anti-CD73 antibody, wherein the anti-CD73 antibody binds the same epitope as a 1E9 antibody; (ii) isolating from the cancer subject a B cell expressing a cancer antigen-binding antibody, and (iii) expressing the gene encoding the cancer antigen-binding antibody, thereby producing a cancer antigen-binding antibody. In embodiments, the gene encoding the cancer antigen-binding antibody is obtained from the isolated B cell in step (ii). In embodiments, the methods comprise isolating the gene from the B cell. In embodiments, the methods comprise isolating the gene from the B cell, and then expressing the gene encoding the cancer antigen-binding antibody. In embodiments, the disclosure provides methods of treating cancer in a subject in need thereof by administering to the subject an effective amount of a cancer antigen-binding antibody. In embodiments, the disclosure provides methods of treating cancer in a subject in need thereof by administering to the subject an effective amount of a cancer antigen-binding antibody, wherein the cancer antigen-binding antibody is produced by the methods described herein. In embodiments, the anti-CD73 antibody is CPI-006. In embodiments, the cancer is renal cancer. In embodiments, the cancer is prostate cancer. In embodiments, the cancer is lung cancer. In embodiments, the cancer is melanoma. In embodiments, the cancer is breast cancer. In embodiments, the cancer is colorectal cancer. In embodiments, the cancer is hepatocellular cancer. In embodiments, the cancer is head and neck cancer. In embodiments, the cancer is lymphoma. In embodiments, the cancer is renal cancer, prostate cancer, lung cancer, melanoma, breast cancer, colorectal cancer, hepatocellular cancer, head and neck cancer or lymphoma.

The term “cancer antigen” refers to peptides, proteins, or fragments thereof expressed on the surface of a cancer cell that are capable of binding to the antibody binding domain provided herein. In embodiments, a cancer antigen is a tumor-associated antigen. In embodiments, a cancer antigen is a tumor-specific antigen. Cancer antigens expressed on the surface of cancer cells include, for example, alpha-fetoprotein, beta-2-microglobulin, beta-human chorionic gonadotropin, calcitonin, bladder tumor antigen, CD117, CA15-3, CA19-9, CA-125, CA 27.29, CA72-4, calcitonin, carcinoembryonic antigen, CD20, CD22, CD25, CD30, CD33, chromogranin A, cytokeratin fragment 21-1, estrogen receptor, progesterone receptor, fibrinogen, gastrin, HE4, Her2/neu, neuron-specific enolase, nuclear matrix protein 22, prostatic acid phosphatase, PD-L1, prostate-specific antigen, somatostatin receptor, thyroglobulin, 5-HIAA, osteocalcin, transferrin receptor, alkaline phosphatase, BRAF, KRAS, NMP22, BRCA2, urokinase plasminogen activator, apoliprotein A1, S100, nestin, cytokeratin fragments 21-1, ferritin, tissue polypeptide antigen, epididymal secretory protein E4, CECAM 5, CECAM 6, serum M-protein, CTLA4, B7-1/B7-2, MUC-1, epithelial tumor antigen, tyrosinase, melanoma-associated antigen, p53, MART-2, p53, and the like. In embodiments, the cancer antigen is alpha-fetoprotein. In embodiments, the cancer antigen is beta-2-microglobulin. In embodiments, the cancer antigen is beta-human chorionic gonadotropin. In embodiments, the cancer antigen is calcitonin. In embodiments, the cancer antigen is bladder tumor antigen. In embodiments, the cancer antigen is CD117. In embodiments, the cancer antigen is CA15-3. In embodiments, the cancer antigen is CA19-9. In embodiments, the cancer antigen is CA-125. In embodiments, the cancer antigen is CA 27.29. In embodiments, the cancer antigen is CA72-4, calcitonin. In embodiments, the cancer antigen is carcinoembryonic antigen. In embodiments, the cancer antigen is CD20. In embodiments, the cancer antigen is CD22. In embodiments, the cancer antigen is CD25. In embodiments, the cancer antigen is CD30. In embodiments, the cancer antigen is CD33, chromogranin A. In embodiments, the cancer antigen is cytokeratin fragment 21-1. In embodiments, the cancer antigen is estrogen receptor. In embodiments, the cancer antigen is progesterone receptor. In embodiments, the cancer antigen is fibrinogen. In embodiments, the cancer antigen is gastrin. In embodiments, the cancer antigen is HE4. In embodiments, the cancer antigen is Her2/neu. In embodiments, the cancer antigen is neuron-specific enolase. In embodiments, the cancer antigen is nuclear matrix protein 22. In embodiments, the cancer antigen is prostatic acid phosphatase. In embodiments, the cancer antigen is PD-L1. In embodiments, the cancer antigen is prostate-specific antigen. In embodiments, the cancer antigen is somatostatin receptor. In embodiments, the cancer antigen is thyroglobulin. In embodiments, the cancer antigen is 5-HIAA. In embodiments, the cancer antigen is osteocalcin. In embodiments, the cancer antigen is transferrin receptor. In embodiments, the cancer antigen is alkaline phosphtase. In embodiments, the cancer antigen is BRAF. In embodiments, the cancer antigen is KRAS. In embodiments, the cancer antigen is NMP22. In embodiments, the cancer antigen is BRCA2. In embodiments, the cancer antigen is urokinase plasminogen activator. In embodiments, the cancer antigen is apoliprotein A1. In embodiments, the cancer antigen is S100. In embodiments, the cancer antigen is nestin. In embodiments, the cancer antigen is cytokeratin fragments 21-1. In embodiments, the cancer antigen is ferritin. In embodiments, the cancer antigen is tissue polypeptide antigen. In embodiments, the cancer antigen is epididymal secretory protein E4. In embodiments, the cancer antigen is CECAM 5. In embodiments, the cancer antigen is CECAM 6. In embodiments, the cancer antigen is serum M-protein. In embodiments, the cancer antigen is CTLA-4. In embodiments, the cancer antigen is B7-1/B7-2. In embodiments, the cancer antigen is MUC-1. In embodiments, the cancer antigen is epithelial tumor antigen. In embodiments, the cancer antigen is tyrosinase. In embodiments, the cancer antigen is melanoma-associated antigen. In embodiments, the cancer antigen is p53. In embodiments, the cancer antigen is MART-2. In embodiments, the cancer antigen is beta-catenin.

In embodiments, the disclosure provides methods of producing a cancer antigen-binding antibody by (i) administering to a cancer subject an effective amount of an anti-CD73 antibody including an 1E9 CDR L1, an 1E9 CDR L2, an 1E9 CDR L3, an 1E9 CDR H1, an 1E9 CDR H2, and an 1E9 CDR H3, (ii) isolating from the cancer subject a B cell expressing a cancer antigen-binding antibody, and (iii) expressing the gene encoding the cancer antigen-binding antibody, thereby producing a cancer antigen-binding antibody. In embodiments, the gene encoding the cancer antigen-binding antibody is obtained from the isolated B cell in step (ii). In embodiments, the methods comprise isolating the gene from the B cell. In embodiments, the methods comprise isolating the gene from the B cell, and then expressing the gene encoding the cancer antigen-binding antibody. In embodiments, the cancer is renal cancer. In embodiments, the cancer is prostate cancer. In embodiments, the cancer is lung cancer. In embodiments, the cancer is melanoma. In embodiments, the cancer is breast cancer. In embodiments, the cancer is colorectal cancer. In embodiments, the cancer is hepatocellular cancer. In embodiments, the cancer is head and neck cancer. In embodiments, the cancer is lymphoma. In embodiments, the cancer is renal cancer, prostate cancer, lung cancer, melanoma, breast cancer, colorectal cancer, hepatocellular cancer, head and neck cancer or lymphoma.

For the methods provided herein the isolating may include one or more B cells from a subject. Thus, in embodiments, the isolating of step (ii) further includes isolating a plurality of B cells from the cancer subject. Isolation of a plurality (i.e., two or more) may be accomplished using cell biology methods well known and commonly used in the art (e.g., cell sorting procedures using cell surface antigen markers). Once the plurality of B cells is isolated from a biological sample of the subject, the plurality of B cells may be further processed by isolating a specific subclass of B cells. Thus, in embodiments, the method further includes isolating from the plurality of B cells a group of B cells expressing CD19. In embodiments, the method further includes isolating from the plurality of B cells a group of B cells expressing CD20. In embodiments, the method further includes isolating from the plurality of B cells a group of B cells expressing CD27. In embodiments, the method further includes isolating from the plurality of B cells a group of B cells expressing CD38. In embodiments, the method further includes isolating from the plurality of B cells a group of B cells expressing CD24. In embodiments, the method further includes isolating from the plurality of B cells a group of B cells expressing CD19, CD20, CD27 or CD38. In embodiments, the method further includes isolating from the plurality of B cells a group of B cells expressing CD19, CD20, CD27 and CD38. In embodiments, the method further includes isolating from the plurality of B cells a group of B cells expressing CD19, CD20, CD27 and/or CD38. In embodiments, the method further includes isolating from the plurality of B cells a group of B cells expressing CD19, CD20, CD27, CD38 or CD24. In embodiments, the method further includes isolating from the plurality of B cells a group of B cells expressing CD19, CD20, CD27, CD38 and CD24. In embodiments, the method further includes isolating from the plurality of B cells a group of B cells expressing CD19, CD20, CD27, CD38 and/or CD24.

In embodiments, the method further includes isolating from the plurality of B cells a group of B cells expressing at least one of CD19, CD20, CD27, CD38 or CD24. In embodiments, the method further includes isolating from the plurality of B cells a group of B cells expressing at least two of CD19, CD20, CD27, CD38 or CD24. In embodiments, the method further includes isolating from the plurality of B cells a group of B cells expressing at least three of CD19, CD20, CD27, CD38 or CD24. In embodiments, the method further includes isolating from the plurality of B cells a group of B cells expressing at least four of CD19, CD20, CD27, CD38 or CD24.

In embodiments, the method further includes isolating from the plurality of B cells group of B cells expressing at least one of CD19, CD38, CD71 or low levels of IgD relative to a standard control. In embodiments, the method further includes isolating from the plurality of B cells a group of B cells expressing at least two of CD19, CD38, CD71 or low levels of IgD relative to a standard control. In embodiments, the method further includes isolating from the plurality of B cells a group of B cells expressing at least three of CD19, CD38, CD71 or low levels of IgD relative to a standard control. In embodiments, the method further includes isolating from the plurality of B cells a group of B cells expressing at least four of CD19, CD38, CD71 or low levels of IgD relative to a standard control. In embodiments, the method further includes isolating from the plurality of B cells group of B cells expressing one of CD19, CD38, CD71 or low levels of IgD relative to a standard control.

In embodiments, the method further includes isolating from the plurality of B cells a group of plasmablast cells expressing at least one of CD19, CD38, CD138 or high levels of CD27 relative to a standard control. In embodiments, the method further includes isolating from the plurality of B cells a group of plasmablast cells expressing one of CD19, CD38, CD138 or high levels of CD27 relative to a standard control. In embodiments, the method further includes isolating from the plurality of B cells a group of plasmablast cells expressing at least two of CD19, CD38, CD138 or very levels of CD27 relative to a standard control. In embodiments, the method further includes isolating from the plurality of B cells a group of plasmablast cells expressing at least three of CD19, CD38, CD138 or high levels of CD27 relative to a standard control. In embodiments, the method further includes isolating from the plurality of B cells a group of plasmablast cells expressing at least four of CD19, CD38, CD138 or high levels of CD27 relative to a standard control.

In embodiments, the method further includes isolating from the plurality of lymphocytes a group of plasma cells expressing at least one of CD38, CD138 or high levels of CD27 relative to a standard control. In embodiments, the method further includes isolating from the plurality of lymphocytes a group of plasma cells expressing one of CD38, CD138 or high levels of CD27 relative to a standard control. In embodiments, the method further includes isolating from the plurality of lymphocytes a group of plasma cells expressing at least two of CD38, CD138 or high levels of CD27 relative to a standard control. In embodiments, the method further includes isolating from the plurality of lymphocytes a group of plasma cells expressing at least three of CD38, CD138 or high levels of CD27 relative to a standard control.

“A high level” or a “low level” in reference to the expression of an antigen (e.g., CD27, IgD) as referred to herein is a level of expression of the antigen expressed by a B cell in a subject or isolated from a subject. A “standard control” as referred to herein refers to a sample that serves as a reference, usually a known reference, for comparison to a test sample. For example, a test sample may be the expression level of the antigen on a pro-B cell, pre-B cell, immature B cell, or a non-B cell. For example, a test sample can be taken from a patient suspected of having cancer or an infectious disease and compared to samples from a known cancer or an infectious disease patient, or a known normal (non-disease) individual. A control can also represent an average value gathered from a population of similar individuals, e.g., cancer or infectious disease patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. In some examples of the disclosed methods, when the expression level of an antigen (e.g., CD27, IgD) is assessed, the level is compared with a control expression level of the antigen (e.g., CD27, IgD). By control expression level is meant the expression level of an antigen (e.g., CD27, IgD) from a sample or subject lacking cancer or an infectious disease, a sample or subject at a selected stage of cancer or cancer state or an infectious disease or an infectious disease state, or in the absence of a particular variable such as a therapeutic agent. Alternatively, the control level comprises a known amount of an antigen (e.g., CD27, IgD). Such a known amount correlates with an average level of subjects lacking cancer or an infectious disease, at a selected stage of cancer or cancer state or an infectious disease or an infectious disease, or in the absence of a particular variable such as a therapeutic agent. A control level also includes the expression level of an antigen (e.g., CD27, IgD) from one or more selected samples or subjects as described herein. For example, a control level includes an assessment of the expression level of an antigen (e.g., CD27, IgD) in a sample from a subject that does not have cancer or an infectious disease, is at a selected stage of cancer or cancer state or an infectious disease or an infectious disease state, or has not received treatment for cancer or an infectious disease. Another exemplary control level includes an assessment of the expression level of an antigen (e.g., CD27, IgD) in samples taken from multiple subjects that do not have cancer or an infectious disease, are at a selected stage of cancer or an infectious disease, or have not received treatment for cancer or an infectious disease.

When the control level includes the expression level of an antigen (e.g., CD27, IgD) in a sample or subject in the absence of a therapeutic agent, the control sample or subject is optionally the same sample or subject to be tested before or after treatment with a therapeutic agent or is a selected sample or subject in the absence of the therapeutic agent. Alternatively, a control level is an average expression level calculated from a number of subjects without a particular disease. A control level also includes a known control level or value known in the art.

In embodiments, the method further includes isolating from the plurality of B cells a group of B cells expressing one or more of CD19, CD20, CD27, CD38 or CD24. In embodiments, the method further includes isolating from the plurality of B cells a group of B cells expressing two or more of CD19, CD20, CD27, CD38 or CD24. In embodiments, the method further includes isolating from the plurality of B cells a group of B cells expressing three or more of CD19, CD20, CD27, CD38 or CD24. In embodiments, the method further includes isolating from the plurality of B cells a group of B cells expressing four or more of CD19, CD20, CD27, CD38 or CD24.

In embodiments, the method further includes isolating from the plurality of B cells a group of B cells expressing one of CD19, CD20, CD27, CD38 or CD24. In embodiments, the method further includes isolating from the plurality of B cells a group of B cells expressing two of CD19, CD20, CD27, CD38 or CD24. In embodiments, the method further includes isolating from the plurality of B cells a group of B cells expressing three of CD19, CD20, CD27, CD38 or CD24. In embodiments, the method further includes isolating from the plurality of B cells a group of B cells expressing four of CD19, CD20, CD27, CD38 or CD24.

The group of B cells isolated from the subject may include B cells of different stages of maturity (e.g., pro-B cell, pre-B cell, immature B cell, mature B cell, plasmablast, plasma cell). In embodiments, the method further includes isolating from the group of B cells a differentiated B cell expressing a cancer antigen-binding antibody. In embodiments, the isolating from the group of B cells includes detecting an expression frequency of the cancer antigen-binding antibody relative to a standard control. The expression frequency may be a level of gene expression of one or more cancer antigen-binding antibodies within the group of B cells. In embodiments, the standard control is the expression level of one or more cancer antigen-binding antibodies within a group of differentiated B cells isolated prior to administering the anti-CD73 antibody to the cancer subject. In embodiments, the standard control is the expression level of one or more cancer antigen-binding antibodies within a group of differentiated B cells isolated from a healthy subject.

In embodiments, the expression frequency is equal to or greater than 0.0001%. In embodiments, the expression frequency is equal to about 0.0001%. In embodiments, the expression frequency is greater than about 0.0001%. In embodiments, the expression frequency is equal to about 1%. In embodiments, the expression frequency is equal to 1%. In embodiments, the expression frequency is more than about 1%. In embodiments, the expression frequency is more than 1%. In embodiments, the expression frequency is equal to or more than about 1%.

In embodiments, the isolating from the group of B cells includes detecting a clonal frequency of B cells expressing a cancer antigen-binding antibody relative to a standard control. A clonal frequency is the amount of B cells expressing the same cancer antigen-binding antibody within a group of differentiated B cells. In embodiments, the standard control is the amount of B cells expressing the same cancer antigen-binding antibody within a group of differentiated B cells isolated prior to administering the anti-CD73 antibody to the cancer subject. In embodiments, the amount of B cells expressing the same cancer antigen-binding antibody within a group of differentiated B cells isolated from a healthy subject.

In embodiments, the clonal frequency is equal to or greater than 0.0001%. In embodiments, the clonal frequency is equal to about 0.0001%. In embodiments, the clonal frequency is greater than about 0.0001%. In embodiments, the clonal frequency is equal to about 1%. In embodiments, the clonal frequency is equal to 1%. In embodiments, the clonal frequency is more than about 1%. In embodiments, the clonal frequency is more than 1%. In embodiments, the clonal frequency is equal to or more than about 1%.

The cancer antigen-binding antibody provided herein may further be tested for its ability to bind to cancer antigens in vitro. Thus, after producing the cancer antigen-binding antibody by expressing the gene encoding it, the cancer antigen-binding antibody may be contacted with a recombinant or naturally occurring cancer antigen or a standard control (antigen that is not detectably bound by the cancer antigen-binding antibody). Thus, in embodiments, the method further includes detecting a level of binding of the cancer antigen-binding antibody to a cancer antigen relative to a standard control. In embodiments, the cancer antigen is a cancer antigen expressed by the cancer subject. In embodiments, the cancer antigen is a cancer antigen expressed by a second cancer subject. In embodiments, the standard control is an antigen expressed by a non-cancer subject. In embodiments, the standard control is a cancer antigen expressed by a second cancer subject.

In another aspect is provided a method of producing an infectious disease antigen-binding antibody. The method includes (i) administering to an infectious disease subject an effective amount of an anti-CD73 antibody, wherein the anti-CD73 antibody binds the same epitope as a 1E9 antibody; (ii) isolating from the cancer subject a B cell expressing an infectious disease antigen-binding antibody, and (iii) expressing the gene encoding the infectious disease antigen-binding antibody, thereby producing an infectious disease antigen-binding antibody. In embodiments, the gene encoding the infectious disease antigen-binding antibody is obtained from the isolated B cell in step (ii). In embodiments, the methods comprise isolating the gene from the B cell. In embodiments, the methods comprise isolating the gene from the B cell, and then expressing the gene encoding the infectious disease antigen-binding antibody. In embodiments, the disclosure provides methods of treating an infectious disease in a subject in need thereof by administering to the subject an effective amount of an infectious disease antigen-binding antibody. In embodiments, the disclosure provides methods of treating a viral infection in a subject in need thereof by administering to the subject an effective amount of a viral infection antigen-binding antibody.

In an aspect is provided a method of producing an infectious disease antigen-binding antibody. The method includes (i) administering to an infectious disease subject an effective amount of an anti-CD73 antibody including an 1E9 CDR L1, an 1E9 CDR L2, an 1E9 CDR L3, an 1E9 CDR H1, an 1E9 CDR H2, and an 1E9 CDR H3, (ii) isolating from the infectious disease subject a B cell expressing an infectious disease antigen-binding antibody, and (iii) expressing the gene encoding the infectious disease antigen-binding antibody, thereby producing an infectious disease antigen-binding antibody. In embodiments, the gene encoding the infectious disease antigen-binding antibody is obtained from the isolated B cell in step (ii). In embodiments, the methods comprise isolating the gene from the B cell. In embodiments, the methods comprise isolating the gene from the B cell, and then expressing the gene encoding the infectious disease antigen-binding antibody.

As described above, for the methods provided herein the isolating may include one or more B cells from a subject. Thus, in embodiments, the isolating of step (ii) further includes isolating a plurality of B cells from the infectious disease subject. Isolation of a plurality (i.e., two or more) may be accomplished using cell biology methods well know and commonly used in the art (e.g., cell sorting procedures using cell surface antigen markers). Once the plurality of B cells is isolated from a biological sample of the subject, the plurality of B cells may be further processed by isolating a specific subclass of B cells. Thus, in embodiments, the method further includes isolating from the plurality of B cells a group of B cells expressing CD19. In embodiments, the method further includes isolating from the plurality of B cells a group of B cells expressing CD20. In embodiments, the method further includes isolating from the plurality of B cells a group of B cells expressing CD27. In embodiments, the method further includes isolating from the plurality of B cells a group of B cells expressing CD38. In embodiments, the method further includes isolating from the plurality of B cells a group of B cells expressing CD24. In embodiments, the method further includes isolating from the plurality of B cells a group of B cells expressing CD19, CD20, CD27 or CD38. In embodiments, the method further includes isolating from the plurality of B cells a group of B cells expressing CD19, CD20, CD27 and CD38. In embodiments, the method further includes isolating from the plurality of B cells a group of B cells expressing CD19, CD20, CD27 and/or CD38. In embodiments, the method further includes isolating from the plurality of B cells a group of B cells expressing CD19, CD20, CD27, CD38 or CD24. In embodiments, the method further includes isolating from the plurality of B cells a group of B cells expressing CD19, CD20, CD27, CD38 and CD24. In embodiments, the method further includes isolating from the plurality of B cells a group of B cells expressing CD19, CD20, CD27, CD38 and/or CD24.

In embodiments, the method further includes isolating from the plurality of B cells a group of B cells expressing at least one of CD19, CD20, CD27, CD38 or CD24. In embodiments, the method further includes isolating from the plurality of B cells a group of B cells expressing one of CD19, CD20, CD27, CD38 or CD24. In embodiments, the method further includes isolating from the plurality of B cells a group of B cells expressing at least two of CD19, CD20, CD27, CD38 or CD24. In embodiments, the method further includes isolating from the plurality of B cells a group of B cells expressing at least three of CD19, CD20, CD27, CD38 or CD24. In embodiments, the method further includes isolating from the plurality of B cells a group of B cells expressing at least four of CD19, CD20, CD27, CD38 or CD24.

In embodiments, the method further includes isolating from the plurality of B cells group of B cells expressing one or more of CD19, CD20, CD27, CD38 or CD24. In embodiments, the method further includes isolating from the plurality of B cells group of B cells expressing two or more of CD19, CD20, CD27, CD38 or CD24. In embodiments, the method further includes isolating from the plurality of B cells group of B cells expressing three or more of CD19, CD20, CD27, CD38 or CD24. In embodiments, the method further includes isolating from the plurality of B cells group of B cells expressing four or more of CD19, CD20, CD27, CD38 or CD24.

In embodiments, the method further includes isolating from the plurality of B cells a group of B cells expressing one of CD19, CD20, CD27, CD38 or CD24. In embodiments, the method further includes isolating from the plurality of B cells a group of B cells expressing two of CD19, CD20, CD27, CD38 or CD24. In embodiments, the method further includes isolating from the plurality of B cells a group of B cells expressing three of CD19, CD20, CD27, CD38 or CD24. In embodiments, the method further includes isolating from the plurality of B cells a group of B cells expressing four of CD19, CD20, CD27, CD38 or CD24.

In embodiments, the method further includes isolating from the plurality of B cells a group of B cells expressing at least one of CD19, CD38, CD71 or low levels of IgD relative to a standard control. In embodiments, the method further includes isolating from the plurality of B cells a group of B cells expressing one of CD19, CD38, CD71 or low levels of IgD relative to a standard control. In embodiments, the method further includes isolating from the plurality of B cells a group of B cells expressing at least two of CD19, CD38, CD71 or low levels of IgD relative to a standard control. In embodiments, the method further includes isolating from the plurality of B cells a group of B cells expressing at least three of CD19, CD38, CD71 or low levels of IgD relative to a standard control. In embodiments, the method further includes isolating from the plurality of B cells a group of B cells expressing at least four of CD19, CD38, CD71 or low levels of IgD relative to a standard control.

In embodiments, the method further includes isolating from the plurality of B cells a group of plasmablast cells expressing at least one of CD19, CD38, CD138 or high levels of CD27 relative to a standard control. In embodiments, the method further includes isolating from the plurality of B cells a group of plasmablast cells expressing one of CD19, CD38, CD138 or high levels of CD27 relative to a standard control. In embodiments, the method further includes isolating from the plurality of B cells a group of plasmablast cells expressing at least two of CD19, CD38, CD138 or high levels of CD27 relative to a standard control. In embodiments, the method further includes isolating from the plurality of B cells a group of plasmablast cells expressing at least three of CD19, CD38, CD138 or high levels of CD27 relative to a standard control. In embodiments, the method further includes isolating from the plurality of B cells a group of plasmablast cells expressing at least four of CD19, CD38, CD138 or high levels of CD27 relative to a standard control.

In embodiments, the method further includes isolating from the plurality of lymphocytes a group of plasma cells expressing at least one of CD38, CD138 or high levels of CD27 relative to a standard control. In embodiments, the method further includes isolating from the plurality of lymphocytes a group of plasma cells expressing one of CD38, CD138 or high levels of CD27 relative to a standard control. In embodiments, the method further includes isolating from the plurality of lymphocytes a group of plasma cells expressing at least two of CD38, CD138 or high levels of CD27 relative to a standard control. In embodiments, the method further includes isolating from the plurality of lymphocytes a group of plasma cells expressing at least three of CD38, CD138 or high levels of CD27 relative to a standard control.

The group of B cells isolated from the subject may include B cells of different stages of maturity (e.g., pro-B cell, pre-B cell, immature B cell, mature B cell, plasmablast, plasma cell). In embodiments, the method further includes isolating from the group of B cells a differentiated B cell expressing an infectious disease antigen-binding antibody. In embodiments, the isolating from the group of B cells includes detecting an expression frequency of the infectious disease antigen-binding antibody relative to a standard control. The expression frequency may be a level of gene expression of one or more infectious disease antigen-binding antibodies within the group of B cells. In embodiments, the standard control is the expression level of one or more infectious disease antigen-binding antibodies within a group of differentiated B cells isolated prior to administering the anti-CD73 antibody to the infectious disease subject. In embodiments, the standard control is the expression level of one or more infectious disease antigen-binding antibodies within a group of differentiated B cells isolated from a healthy subject.

In embodiments, the expression frequency is equal to or greater than 0.0001%. In embodiments, the expression frequency is equal to about 0.0001%. In embodiments, the expression frequency is greater than about 0.0001%. In embodiments, the expression frequency is equal to about 1%. In embodiments, the expression frequency is equal to 1%. In embodiments, the expression frequency is more than about 1%. In embodiments, the expression frequency is more than 1%. In embodiments, the expression frequency is equal to or more than about 1%.

In embodiments, the isolating from the group of B cells includes detecting a clonal frequency of B cells expressing an infectious disease antigen-binding antibody relative to a standard control. A clonal frequency is the amount of B cells expressing the same infectious disease antigen-binding antibody within a group of differentiated B cells. In embodiments, the standard control is the amount of B cells expressing the same infectious disease antigen-binding antibody within a group of differentiated B cells isolated prior to administering the anti-CD73 antibody to the infectious disease subject. In embodiments, the amount of B cells expressing the same infectious disease antigen-binding antibody within a group of differentiated B cells isolated from a healthy subject.

In embodiments, the clonal frequency is equal to or greater than 0.0001%. In embodiments, the clonal frequency is equal to about 0.0001%. In embodiments, the clonal frequency is greater than about 0.0001%. In embodiments, the clonal frequency is equal to about 1%. In embodiments, the clonal frequency is equal to 1%. In embodiments, the clonal frequency is more than about 1%. In embodiments, the clonal frequency is more than 1%. In embodiments, the clonal frequency is equal to or more than about 1%.

The infectious disease antigen-binding antibody provided herein may further be tested for its ability to bind to infectious disease antigens in vitro. Thus, after producing the infectious disease antigen-binding antibody by expressing the gene expressing it, the infectious disease antigen-binding antibody may be contacted with a recombinant or naturally occurring infectious disease antigen or a standard control (antigen that is not detectably bound by the infectious disease antigen-binding antibody). Thus, in embodiments, the method further includes detecting a level of binding of the infectious disease antigen-binding antibody to an infectious disease antigen relative to a standard control. In embodiments, the infectious disease antigen is an infectious disease antigen expressed by the infectious disease subject. In embodiments, the infectious disease antigen is an infectious disease antigen expressed by a second infectious disease subject. In embodiments, the standard control is an antigen expressed by a non-infectious disease subject. In embodiments, the standard control is an infectious disease antigen expressed by a second infectious disease subject.

In embodiments, the B cell is a differentiated B cell. In embodiments, the B cell expresses CD19. In embodiments, the B cell expresses CD20. In embodiments, the B cell expresses CD27. In embodiments, the B cell expresses CD38. In embodiments, the B cell expresses CD71. In embodiments, the B cell expresses low levels of IgD relative to a standard control. In embodiments, the B cell expressing CD19, CD20, CD27 or CD38. In embodiments, the B cell expressing CD19, CD20, CD27 and CD38. In embodiments, the B cell expressing CD19, CD38, CD71 and low levels of IgD relative to a standard control.

In embodiments, the B cell is isolated from a blood sample of the subject. In embodiments, the B cell is isolated from a lymphoid tissue sample of the subject. In embodiments, the B cell is isolated from a bone marrow sample of the subject. In embodiments, the B cell is isolated from a blood sample, a lymphoid tissue sample or a bone marrow sample of the subject.

The antibodies formed by the methods provided herein including embodiments thereof, may be, inter alia, used for therapeutic or diagnostic purposes. Thus, in an aspect is provided a method of treating cancer in a subject in need thereof. The method includes administering to the subject an effective amount of a cancer antigen-binding antibody formed by the methods provided herein including embodiments thereof. In embodiments, the cancer is renal cancer. In embodiments, the cancer is prostate cancer. In embodiments, the cancer is lung cancer. In embodiments, the cancer is melanoma. In embodiments, the cancer is breast cancer. In embodiments, the cancer is colorectal cancer. In embodiments, the cancer is hepatocellular cancer. In embodiments, the cancer is head and neck cancer. In embodiments, the cancer is lymphoma. In embodiments, the cancer is renal cancer, prostate cancer, lung cancer, melanoma, breast cancer, colorectal cancer, hepatocellular cancer, head and neck cancer or lymphoma.

The methods of producing antibodies provided herein including embodiments thereof, may be used for treatment purposes (e.g., treatment of cancer, infectious disease, viral infection). In embodiments, the antibodies may be administered to the subject they were formed in or they may be administered to another subject. Thus, in an aspect is provided a method of treating cancer in a subject in need thereof. The method includes (i) administering to a first cancer subject an effective amount of an anti-CD73 antibody including an 1E9 CDR L1, an 1E9 CDR L2, an 1E9 CDR L3, an 1E9 CDR H1, an 1E9 CDR H2, and an 1E9 CDR H3, (ii) isolating from the first cancer subject a B cell expressing a cancer antigen-binding antibody, (iii) expressing the gene encoding the cancer antigen-binding antibody, thereby forming an isolated cancer antigen-binding antibody, and (iv) administering the isolated cancer antigen-binding antibody to a second cancer subject, thereby treating cancer in a subject. In embodiments, the gene encoding the cancer antigen-binding antibody is obtained from the isolated B cell in step (ii). In embodiments, the methods comprise isolating the gene from the B cell. In embodiments, the methods comprise isolating the gene from the B cell, and then expressing the gene encoding the cancer antigen-binding antibody. In embodiments, the first cancer subject and the second cancer subject are the same. In embodiments, the first subject and the second cancer subject are different. In embodiments, the antibody is formed in the first subject, isolated from the first subject, and then administered to a different subject expressing the same cancer antigen. In embodiments, the cancer is renal cancer, prostate cancer, lung cancer, melanoma, breast cancer, colorectal cancer, hepatocellular cancer, head and neck cancer or lymphoma.

In another aspect is provided a method of treating an infectious disease in a subject in need thereof. In embodiments, the infectious disease is a viral infection. The method includes (i) administering to a first infectious disease subject an effective amount of an anti-CD73 antibody including an 1E9 CDR L1, an 1E9 CDR L2, an 1E9 CDR L3, an 1E9 CDR H1, an 1E9 CDR H2, and an 1E9 CDR H3. The method further includes (ii) isolating from the first infectious disease subject a B cell expressing an infectious disease-binding antibody, (iii) expressing the gene encoding the infectious disease-binding antibody, thereby forming an isolated infectious disease-binding antibody, and (iv) administering the isolated infectious disease-binding antibody to a second infectious disease subject, thereby treating an infectious disease in a subject. In embodiments, the gene encoding the infectious disease antigen-binding antibody is obtained from the isolated B cell in step (ii). In embodiments, the methods comprise isolating the gene from the B cell. In embodiments, the methods comprise isolating the gene from the B cell, and then expressing the gene encoding the infectious disease antigen-binding antibody. In embodiments, the first infectious disease subject and the second infectious disease subject are the same. In embodiments, the antibody is formed in the first subject, isolated from the first subject, and then administered to a different subject having the same infectious disease. In embodiments, the first infectious disease subject and the second infectious disease subject are different. In embodiments, the viral infection is COVID-19, MERS, bronchiolitis, common cold, croup, influenza, pneumonia, norovirus, rotavirus, adenovirus, astrovirus, measles, mumps, chicken pox, rubella, chickenpox, shingles, roseola, smallpox, fifth disease, chikungunya, hepatitis A, hepatitis B, hepatitis C, hepatitis D, hepatitis E, warts, genital warts, herpes simplex, genital herpes, molluscum contagiosum, Ebola, lassa fever, dengue fever, yellow fever, marburg hemorrhagic fever, Crimean-Congo hemorrhagic fever, polio, viral meningitis, viral encephalitis, rabies, Zika virus, West Nile virus, malaria, or tuberculosis. In embodiments, the viral infection is COVID-19. In embodiments, the viral infection is MERS. In embodiments, the viral infection is SARS-CoV. In embodiments, the viral infection is SARS-CoV-1. In embodiments, the viral infection is SARS-CoV-2. In embodiments, the viral infection is MERS-CoV.

As described above the methods and compositions provided herein may be used, inter alia, for diagnostic purposes. For example, the cancer antigen-binding antibody or the infectious disease antigen-binding antibody may be used to detect a corresponding antigen in a subject in need thereof. Thus, in an aspect is provided a method of detecting a cancer antigen. The method includes (i) contacting a cancer antigen with a cancer antigen-binding antibody formed by methods provided herein including embodiments thereof, and (ii) detecting binding of the cancer antigen-binding antibody to the cancer antigen, thereby detecting a cancer antigen. In another aspect is provided a method of detecting an infectious disease antigen. The method includes (i) contacting an infectious disease antigen with an infectious disease antigen-binding antibody formed by methods provided herein including embodiments thereof, and (ii) detecting binding of the infectious disease antigen-binding antibody to the infectious disease antigen, thereby detecting an infectious disease antigen.

### Pharmaceutical Compositions

The anti-CD73 antibody provided herein including embodiments thereof may be used for vaccination or immunization purposes to cancer or infectious disease subject. In embodiments, the anti-CD73 antibody is an adjuvant. In an aspect is provided a pharmaceutical composition including an effective amount of an anti-CD73 antibody including an 1E9 CDR L1, an 1E9 CDR L2, an 1E9 CDR L3, an 1E9 CDR H1, an 1E9 CDR H2, and an 1E9 CDR H3, an adjuvant and a pharmaceutical excipient. In an aspect is provided a pharmaceutical composition comprising CPI-006.

In an aspect is provided a pharmaceutical composition including an effective amount of an anti-CD73 antibody including an 1E9 CDR L1, an 1E9 CDR L2, an 1E9 CDR L3, an 1E9 CDR H1, an 1E9 CDR H2, and an 1E9 CDR H3, antigenic peptide and a pharmaceutical excipient. In embodiments, the antigenic peptide is a cancer-associated immunogenic lysate, protein, or peptide. In embodiments, the antigenic peptide is a neoantigen peptide. In embodiments, the antigenic peptide is from an infectious agent. In embodiments, the antigenic peptide is live-attenuated, killed or fragment of a pathogenic virus, bacteria, fungus, or parasite. In embodiments, the antigenic peptide is live-attenuated, killed or fragment of a pathogenic virus. In embodiments, the antigenic peptide is live-attenuated, killed or fragment of SARS-CoV. In embodiments, the antigenic peptide is live-attenuated, killed or fragment of SARS-CoV-2. In embodiments, the antigenic peptide is live-attenuated, killed or fragment of MERS-CoV. In embodiments, the antigenic peptide is an influenza virus or a functional fragment thereof. In embodiments, the antigenic peptide is a cancer peptide. A “cancer peptide” as provided herein refers to a peptide derived from a cancer cell. In embodiments, the antigenic peptide is an infectious disease peptide. An “infectious disease peptide” as provided herein refers to a peptide derived from an infectious agent (e.g., a virus, bacteria).

In embodiments, the adjuvant is a toll-like receptor (TLR) agonist. In embodiments, the adjuvant is an agonist of TLR1/2, TLR3, TLR4, TLR5, TLR2/6, TLR7, TLR8, TLR9, or a subset thereof. In embodiments, the adjuvant is a cancer-associated immunogenic lysate, protein, or peptide. In embodiments, the adjuvant is a neoantigen peptide. In embodiments, the adjuvant is from an infectious agent. In embodiments, the adjuvant is live-attenuated, killed or fragment of a pathogenic virus, bacteria, fungus, or parasite. In embodiments, the adjuvant is live-attenuated, killed or fragment of a virus. In embodiments, the adjuvant is an influenza virus or a functional fragment thereof. In embodiments, the adjuvant is SARS-CoV or a functional fragment thereof. In embodiments, the adjuvant is SARS-CoV-2 or a functional fragment thereof. In embodiments, the adjuvant is SARS-CoV-1 or a functional fragment thereof. In embodiments, the adjuvant is MERS-CoV or a functional fragment thereof.

For vaccination or immunization purposes the anti-CD73 antibody provided herein including embodiments thereof, may be formulated and introduced as a vaccine through oral, intradermal, intramuscular, intraperitoneal, intravenous, subcutaneous, intranasal, and via scarification (scratching through the top layers of skin, e.g., using a bifurcated needle) or any other standard route of immunization. Vaccine formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient or any other oral composition as listed above. Alternatively, the vaccines may be administered parenterally as injections (intravenous, intramuscular or subcutaneous). The amount of anti-CD73 antibody used in a vaccine can depend upon a variety of factors including the route of administration, species, and use of booster administration. However, a person of ordinary skill in the art would immediately recognize appropriate and/or equivalent doses looking at dosages of approved whopping cough vaccines for guidance.

The term “adjuvant” refers to a compound that when administered in conjunction with the anti-CD73 antibody provided herein including embodiments thereof augments the immune response to the antigen, but when administered alone does not generate an immune response to the antigen. As described above the anti-CD73 antibody provided herein including embodiments thereof may be used as an adjuvant. Therefore, the term “adjuvant” refers to a compound that when administered in conjunction with a vaccine (e.g., cancer antigen) augments the immune response to the antigen, but when administered alone does not generate an immune response to the antigen. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the anti-CD73 antibody provided herein including embodiments thereof, to elicit an immune response. Preferred adjuvants augment the intrinsic response to an immunogen without causing conformational changes in the immunogen that affect the qualitative form of the response. Other adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, N Y, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

In embodiments, the adjuvant is an aluminum salt (alum), such as alum hydroxide, alum phosphate, alum sulfate. Such adjuvants can be used with or without other specific immunostimulating agents such as MPL or 3-DMP, QS-21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-A1-D-isoglu-L-Ala-dipalmitoxy propylamide (DTP-DPP) theramideTM), or other bacterial cell wall components. Oil-in-water emulsions include (a) MF59, containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton MA), (b) SAF, containing 10% Squalene, 0.4% Tween 80, 5% pluronic-blocked polymer L121, and thr-MDP, either microfluidized into a submicron emulsion or vortexed to generate a larger particle size emulsion, and (c) Ribi™ adjuvant system (RAS), (Ribi ImmunoChem, Hamilton, MT) containing 2% squalene, 0.2% Tween 80, and one or more bacterial cell wall components from the group consisting of monophosphoryllipid A (MPL), trehalose dimycolate (TDM), and cell wall skeleton (CWS), preferably MPL+CWS (Detox™).

Other adjuvants include saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, . . . , IL-2, IL-4, IL-6, IL-12, IL13, and IL-15), macrophage colony stimulating factor (M-CSF), granulocyte-macrophage colony stimulating factor (GM-CSF), tumor necrosis factor (TNF), chemokines, such as MIP1α and β RANTES. Another class of adjuvants is glycolipid analogues including N-glycosylamides, N-glycosylureas and N-glycosylcarbamates, each of which is substituted in the sugar residue by an amino acid, as immuno-modulators or adjuvants. Heat shock proteins, e.g., HSP70 and HSP90, may also be used as adjuvants.

An adjuvant can be administered with an immunogen as a single composition, or can be administered before, concurrent with or after administration of the immunogen. Immunogen and adjuvant can be packaged and supplied in the same vial or can be packaged in separate vials and mixed before use. Immunogen and adjuvant are typically packaged with a label indicating the intended therapeutic application. If immunogen and adjuvant are packaged separately, the packaging typically includes instructions for mixing before use. The choice of an adjuvant and/or carrier depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Alum, MPL and QS-21 are preferred. Optionally, two or more different adjuvants can be used simultaneously. Preferred combinations include alum with MPL, alum with QS-21, MPL with QS-21, MPL or RC-529 with GM-CSF, and alum, QS-21 and MPL together. Also, Incomplete Freund's adjuvant can be used (Chang et al., Advanced Drug Delivery Reviews 32, 173-186 (1998)), optionally in combination with any of alum, QS-21, and MPL and all combinations thereof.

Agents for inducing an immune response can be administered by parenteral, topical, intravenous, oral, subcutaneous, intraarterial, intracranial, intraperitoneal, intranasal or intramuscular means for prophylactic and/or therapeutic treatment. The most typical route of administration of an immunogenic agent is subcutaneous although other routes can be equally effective. The next most common route is intramuscular injection. This type of injection is most typically performed in the arm or leg muscles.

For parenteral administration, antibodies described herein can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/ml, formulated in aqueous buffer consisting of 50 mM L-histidine, 150 mM NaCl, adjusted to pH 6.0 with HCl. Composition for parenteral administration are typically substantially sterile, isotonic and manufactured under GMP conditions of the FDA or similar body.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). The antibodies described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein. Alternatively, transdermal delivery can be achieved using a skin path or using transferosomes (Paul et al., Eur. J. Immunol. 25, 3521-24 (1995); Cevc et al., Biochem. Biophys. Acta 1368, 201-15 (1998)).

Pharmaceutical compositions include compositions wherein the active ingredient (e.g. described herein and compositions described herein, including embodiments or examples) is contained in a therapeutically effective amount, i.e., in an amount effective to achieve its intended purpose. The actual amount effective for a particular application will depend, inter alia, on the condition being treated and the desired result, e.g., modulating the activity of a target molecule, and/or reducing, eliminating, or slowing the progression of disease symptoms. Determination of a therapeutically effective amount of the antibodies described herein is well within the capabilities of those skilled in the art, especially in light of the detailed disclosure herein.

The compositions described herein can include a single agent or more than one agent. The compositions for administration will commonly include an antibody as described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the subject's needs.

Solutions of the antibodies as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.10% of active compound. The percentage of the compositions and preparations may, of course, be varied and may conveniently be between about 2 to about 75% of the weight of the unit, or preferably between 25-60%. The amount of active compounds in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds or constructs in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Solvents (e.g., dimethyl sulfoxide) can be used for rapid penetration, delivering high concentrations of the active agents to a small area.

The compositions of antibodies can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods and antibodies described herein. Adjustment and manipulation of established dosages (e.g., frequency and duration) are well within the ability of those skilled in the art.

For any composition (e.g., the anti-CD73 antibody provided) described herein, the therapeutically effective amount can be initially determined from cell culture assays. Target concentrations will be those concentrations of active compound(s) that are capable of achieving the methods described herein, as measured using the methods described herein or known in the art. As is well known in the art, effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects.

In embodiments, the anti-CD73 inhibitor is administered to subjects in an effective amount to treat the disease in the subject. In embodiments, the effective amount is from about 0.1 mg/kg to about 10.0 mg/kg. In embodiments, the effective amount is from about 0.2 mg/kg to about 6 mg/kg. In embodiments, the effective amount is from about 0.25 mg/kg to about 5.5 mg/kg. In embodiments, the effective amount is from about 0.3 mg/kg to about 5.0 mg/kg. In embodiments, the effective amount is from about 0.25 mg/kg to about 0.35 mg/kg. In embodiments, the effective amount is 0.3 mg/kg. In embodiments, the effective amount is from about 0.5 mg/kg to about 1.5 mg/kg. In embodiments, the effective amount is 1.0 mg/kg. In embodiments, the effective amount is from about 2 mg/kg to about 4 mg/kg. In embodiments, the effective amount is 3.0 mg/kg. In embodiments, the effective amount is from about 4 mg/kg to about 6 mg/kg. In embodiments, the effective amount is 5.0 mg/kg.

In embodiments, the effective amount is about 1 mg/kg, 3 mg/kg, 6 mg/kg, 10 mg/kg, 30 mg/kg, 40 mg/kg, or 120 mg/kg. In embodiments, the effective amount is about 0.05 mg/kg, 0.1 mg/kg, 0.3 mg/kg, 1 mg/kg, 3 mg/kg, 6 mg/kg, 10 mg/kg, 30 mg/kg, 40 mg/kg, or 120 mg/kg. In embodiments, the effective amount is about 0.05 mg/kg. In embodiments, the effective amount is 0.05 mg/kg. In embodiments, the effective amount is about 0.1 mg/kg. In embodiments, the effective amount is 0.1 mg/kg. In embodiments, the effective amount is about 0.3 mg/kg. In embodiments, the effective amount is 0.3 mg/kg. In embodiments, the effective amount is about 1 mg/kg. In embodiments, the effective amount is 1 mg/kg. In embodiments, the effective amount is about 3 mg/kg. In embodiments, the effective amount is 3 mg/kg. In embodiments, the effective amount is about 6 mg/kg. In embodiments, the effective amount is 6 mg/kg. In embodiments, the effective amount is about 10 mg/kg. In embodiments, the effective amount is 10 mg/kg. In embodiments, the effective amount is about 12 mg/kg. In embodiments, the effective amount is 12 mg/kg. In embodiments, the effective amount is about 30 mg/kg. In embodiments, the effective amount is 30 mg/kg. In embodiments, the effective amount is about 40 mg/kg. In embodiments, the effective amount is 40 mg/kg. In embodiments, the effective amount is about 120 mg/kg. In embodiments, the effective amount is 120 mg/kg. In embodiments, the effective amount administered results in serum levels of the antibody of about 10 μg/ml.

In embodiments, the anti-CD73 antibody is administered to a subject in an effective amount of at least 1 mg/kg. In embodiments, the effective amount is at least 2 mg/kg. In embodiments, the effective amount is at least 2 mg/kg. In embodiments, the effective amount is at least 3 mg/kg. In embodiments, the effective amount is at least 4 mg/kg. In embodiments, the effective amount is at least 5 mg/kg. In embodiments, the effective amount is at least 6 mg/kg. In embodiments, the effective amount is at least 7 mg/kg. In embodiments, the effective amount is at least 8 mg/kg. In embodiments, the effective amount is at least 9 mg/kg. In embodiments, the effective amount is at least 10 mg/kg. In embodiments, the effective amount is at least 11 mg/kg. In embodiments, the effective amount is at least 12 mg/kg. In embodiments, the effective amount is at least 13 mg/kg. In embodiments, the effective amount is at least 14 mg/kg. In embodiments, the effective amount is at least 15 mg/kg. In embodiments, the effective amount is from about 1 mg/kg to about 100 mg/kg. In embodiments, the effective amount is from about 2 mg/kg to about 90 mg/kg. In embodiments, the effective amount is from about 3 mg/kg to about 80 mg/kg. In embodiments, the effective amount is from about 4 mg/kg to about 70 mg/kg. In embodiments, the effective amount is from about 5 mg/kg to about 60 mg/kg. In embodiments, the effective amount is from about 6 mg/kg to about 50 mg/kg. In embodiments, the effective amount is from about 4 mg/kg to about 25 mg/kg. In embodiments, the effective amount is from about 5 mg/kg to about 25 mg/kg. In embodiments, the effective amount is from about 6 mg/kg to about 25 mg/kg. In embodiments, the effective amount is from about 7 mg/kg to about 25 mg/kg. In embodiments, the effective amount is from about 8 mg/kg to about 25 mg/kg. In embodiments, the effective amount is from about 9 mg/kg to about 25 mg/kg. In embodiments, the effective amount is from about 10 mg/kg to about 25 mg/kg. In embodiments, the effective amount is from about 5 mg/kg to about 15 mg/kg. In embodiments, the effective amount is from about 6 mg/kg to about 12 mg/kg. In embodiments, the effective amount is about 4 mg/kg. In embodiments, the effective amount is about 5 mg/kg. In embodiments, the effective amount is about 6 mg/kg. In embodiments, the effective amount is about 7 mg/kg. In embodiments, the effective amount is about 8 mg/kg. In embodiments, the effective amount is about 9 mg/kg. In embodiments, the effective amount is about 10 mg/kg. In embodiments, the effective amount is about 11 mg/kg. In embodiments, the effective amount is about 12 mg/kg. In embodiments, the effective amount is about 13 mg/kg. In embodiments, the effective amount is about 14 mg/kg. In embodiments, the effective amount is about 15 mg/kg. In embodiments, the effective amount is about 16 mg/kg. In embodiments, the effective amount is about 17 mg/kg. In embodiments, the effective amount is about 18 mg/kg. In embodiments, the effective amount is about 19 mg/kg. In embodiments, the effective amount is about 20 mg/kg. In embodiments, the effective amount is about 21 mg/kg. In embodiments, the effective amount is about 22 mg/kg. In embodiments, the effective amount is about 23 mg/kg. In embodiments, the effective amount is about 24 mg/kg. In embodiments, the effective amount is about 25 mg/kg.

In embodiments, the anti-CD73 antibody is administered by parenteral injection. In embodiments, the injection is a bolus injection. In embodiments, the injection is an infusion (e.g., over the course of 5 minutes to 2 hours; or from about 30 minutes to about 90 minutes). In embodiments, the anti-CD73 antibody is administered once per week (i.e., once every 7 days), once every two weeks (e.g., once every 14 days), once every three weeks (e.g., once every 21 days), or once per month (e.g., once every 28 days).

In embodiments for the methods provided herein the anti-CD73 antibody may be administered at a half maximal effective concentration (EC₅₀) of at least 100 nM (e.g., 100, 105, 110, 115, 120, 125, 130, 135, 140, 145, 150, 155, 160, 165, 170, 175, 180, 185, 190, 195, 200, 210, 220, 230, 240, 250 nM). In embodiments, the anti-CD73 antibody is administered at a half maximal effective concentration (EC₅₀) of at least 100 nM. In embodiments, the antibody is administered at a half maximal effective concentration (EC₅₀) of 110 nM. In embodiments, the antibody is administered at a half maximal effective concentration (EC₅₀) of 115 nM. In embodiments, the antibody is administered at a half maximal effective concentration (EC₅₀) of 120 nM. In embodiments, the antibody is administered at a half maximal effective concentration (EC₅₀) of 125 nM. In embodiments, the antibody is administered at a half maximal effective concentration (EC₅₀) of 130 nM. In embodiments, the antibody is administered at a half maximal effective concentration (EC₅₀) of 135 nM. In embodiments, the antibody is administered at a half maximal effective concentration (EC₅₀) of 140 nM. In embodiments, the antibody is administered at a half maximal effective concentration (EC₅₀) of 145 nM. In embodiments, the antibody is administered at a half maximal effective concentration (EC₅₀) of 150 nM. In embodiments, the antibody is administered at a half maximal effective concentration (EC₅₀) of 155 nM. In embodiments, the antibody is administered at a half maximal effective concentration (EC₅₀) of 160 nM. In embodiments, the antibody is administered at a half maximal effective concentration (EC₅₀) of 165 nM. In embodiments, the antibody is administered at a half maximal effective concentration (EC₅₀) of 170 nM. In embodiments, the antibody is administered at a half maximal effective concentration (EC₅₀) of 175 nM. In embodiments, the antibody is administered at a half maximal effective concentration (EC₅₀) of 180 nM. In embodiments, the antibody is administered at a half maximal effective concentration (EC₅₀) of 185 nM. In embodiments, the antibody is administered at a half maximal effective concentration (EC₅₀) of 190 nM. In embodiments, the antibody is administered at a half maximal effective concentration (EC₅₀) of 195 nM. In embodiments, the antibody is administered at a half maximal effective concentration (EC₅₀) of 200 nM. In embodiments, the antibody is administered at a half maximal effective concentration (EC₅₀) of 210 nM. In embodiments, the antibody is administered at a half maximal effective concentration (EC₅₀) of 220 nM. In embodiments, the antibody is administered at a half maximal effective concentration (EC₅₀) of 230 nM. In embodiments, the antibody is administered at a half maximal effective concentration (EC₅₀) of 240 nM. In embodiments, the antibody is administered at a half maximal effective concentration (EC₅₀) of 250 nM.

In embodiments, the antibody is administered at an EC₅₀ of about 137 nM. In embodiments, the antibody is administered at an EC₅₀ of 137 nM. In embodiments, the antibody is administered at an EC₅₀ of about 189 nM. In embodiments, the antibody is administered at an EC₅₀ of 189 nM.

“Pharmaceutically acceptable excipient” and “pharmaceutically acceptable carrier” refer to a substance that aids the administration of an active agent to and absorption by a subject and can be included in pharmaceutical compositions without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like. One of skill in the art will recognize that other pharmaceutical excipients can be used in the pharmaceutical compositions described herein.

In embodiments, the pharmaceutically acceptable carrier is an immunological adjuvant. In some examples, the immunological adjuvant can include, but is not limited to, agonists of Toll-like Receptors (TLRs), agonists of the STING pathway, agonistic antibodies against CD40, OX40, CTLA4, PD1, or PD1-L, Freund's adjuvant, bryostatins and ligands for CD40, OX40, CD137, PD1, CTLA4 and any combinations thereof. In embodiments, the adjuvant can increase immunogenicity that is induced when a cell-penetrating complex by co-administered with the complex to a subject.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

### Embodiments 1-41

Embodiment 1. A method of treating COVID-19 in a subject in need thereof, the method comprising administering to the subject an effective amount of an anti-CD73 antibody.

Embodiment 2. A method of treating severe acute respiratory syndrome (SARS) in a subject in need thereof, the method comprising administering to the subject an effective amount of an anti-CD73 antibody.

Embodiment 3. A method of improving humoral immune response to a severe acute respiratory syndrome (SARS) in a subject in need thereof, the method comprising administering to the subject an effective amount of an anti-CD73 antibody.

Embodiment 4. A method of increasing serum and/or plasma immunoglobulin anti-severe acute respiratory syndrome (SARS) levels in a subject in need thereof, the method comprising administering to the subject an effective amount of an anti-CD73 antibody.

Embodiment 5. The method of any one of Embodiments 2 to 4, wherein the SARS is a SARS-coronavirus.

Embodiment 6. The method of Embodiment 5, wherein the SARS-coronavirus is SARS-coronavirus 2.

Embodiment 7. The method of Embodiment 5, wherein the SARS-coronavirus is SARS-coronavirus 2.

Embodiment 8. The method of Embodiment 5, wherein the SARS-coronavirus is SARS-coronavirus 2.

Embodiment 9. The method of any one of Embodiments 1 to 8, wherein the anti-CD73 antibody is a humanized FcγR binding-deficient monoclonal antibody.

Embodiment 10. The method of any one of Embodiments 1 to 8, wherein the anti-CD73 antibody comprises 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 11. The method of any one of Embodiments 1 to 8, wherein the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and the CDR H3 has a sequence of SEQ ID NO:6.

Embodiment 12. The method of any one of Embodiments 1 to 11, wherein the anti-CD73 antibody comprises a humanized light chain variable region and a humanized heavy chain variable region, wherein the humanized light chain variable region comprises a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87; and wherein the humanized heavy chain variable region comprises an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

Embodiment 13. The method of Embodiment 12, wherein the humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

Embodiment 14. The method of Embodiment 12 or 13, wherein the humanized heavy chain variable region comprises a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

Embodiment 15. The method of any one of Embodiments 1 to 8, wherein the anti-CD73 antibody comprises a light chain variable region having SEQ ID NO:8 and a heavy chain variable region having SEQ ID NO:7.

Embodiment 16. The method of any one of Embodiments 1 to 8, wherein the anti-CD73 antibody comprises a light chain having SEQ ID NO:10 and a heavy chain having SEQ ID NO:9.

Embodiment 17. The method of any one of Embodiments 1 to 16, wherein the anti-CD73 antibody is an IgG.

Embodiment 18. The method of any one of Embodiments 1 to 16, wherein the anti-CD73 antibody is an IgG1.

Embodiment 19. The method of any one of Embodiments 1 to 16, wherein the anti-CD73 antibody is an IgG4.

Embodiment 20. The method of any one of Embodiments 1 to 19, wherein the anti-CD73 antibody is a Fab′ fragment.

Embodiment 21. The method of any one of Embodiments 1 to 19, wherein the anti-CD73 antibody is a single chain antibody (scFv).

Embodiment 22. The method of any one of Embodiments 1 to 21, wherein the anti-CD73 antibody is capable of binding a CD73 antigen with an equilibrium dissociation constant (K_{D}) from about 0.3 to about 25 nM.

Embodiment 23. The method of Embodiment 22, wherein the anti-CD73 antibody is capable of binding a CD73 antigen with an equilibrium dissociation constant (K_{D}) of about 0.64 nM.

Embodiment 24. The method of any one of Embodiments 1 to 23, wherein the anti-CD73 antibody is capable of binding a CD73 antigen at a pH of less than about 7.5.

Embodiment 25. The method of Embodiment 24, wherein the anti anti-CD73 antibody body is capable of binding a CD73 antigen at a pH from about 6.0 to about 7.0.

Embodiment 26. The method of Embodiments 25, wherein the anti-CD73 antibody is capable of binding a CD73 antigen at a pH of about 6.3.

Embodiment 27. The method of any one of Embodiments 1 to 26, wherein the anti-CD73 antibody further comprises a glutamine at a position corresponding to Kabat position 297.

Embodiment 28. The method of any one of Embodiments 1 to 8, wherein the anti-CD73 antibody is oleclumab, BMS-986179, IPH5301, or AD2.

Embodiment 29. The method of any one of Embodiments 1 to 28, wherein the anti-CD73 antibody is bound to a CD73 antigen.

Embodiment 30. The method of Embodiment 29, wherein the CD73 antigen forms part of a cell.

Embodiment 31. The method of Embodiment 30, wherein the cell is a lymphoid cell.

Embodiment 32. The method of Embodiment 30, wherein the cell is a B cell.

Embodiment. 33. The method of any one of Embodiments 1 to 32, comprising parenterally administering the anti-CD73 antibody.

Embodiment. 34. The method of any one of Embodiments 1 to 32, comprising intravenously administering the anti-CD73 antibody.

Embodiment. 35. The method of any one of Embodiments 1 to 32, comprising subcutaneously administering the anti-CD73 antibody.

Embodiment 36. The method of any one of Embodiments 1 to 35, wherein the subject is asymptomatic.

Embodiment 37. The method of any one of Embodiments 1 to 35, wherein the subject is mildly symptomatic.

Embodiment 38. The method of any one of Embodiments 1 to 35, wherein the subject is moderately symptomatic.

Embodiment 39. The method of any one of Embodiments 1 to 35, wherein the subject is severely symptomatic.

Embodiment 40. The method of any one of Embodiments 1 to 39, further comprising administering a vaccine, an anti-viral agent or a combination thereof to the subject.

Embodiment 41. The method of any one of Embodiments 1 to 40, wherein the effective amount is from about 0.1 mg/kg to about 10 mg/kg.

### Embodiments P1 to P56

Embodiment P1. A method of producing a cancer antigen-binding antibody, the method comprising: (i) administering to a cancer subject an effective amount of an anti-CD73 antibody comprising a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3; (ii) isolating from the cancer subject a B cell expressing a cancer antigen-binding antibody; and (iii) expressing the gene encoding the cancer antigen-binding antibody, thereby producing a cancer antigen-binding antibody.

Embodiment P2. The method of Embodiment P1, wherein the isolating of step (ii) further comprises isolating a plurality of B cells from the cancer subject.

Embodiment P3. The method of Embodiment P2, further comprising isolating from the plurality of B cells a group of B cells expressing CD19, CD20, CD27 and/or CD38

Embodiment P4. The method of Embodiment P3, further comprising isolating from the group of B cells a differentiated B cell expressing a cancer antigen-binding antibody.

Embodiment P5. The method of any one of Embodiments P1-P4, further comprising detecting a level of binding of the cancer antigen-binding antibody to a cancer antigen relative to a standard control.

Embodiment P6. The method of Embodiment P5, wherein the cancer antigen is a cancer antigen expressed by the cancer subject

Embodiment P7. The method of Embodiment P5, wherein the cancer antigen is a cancer antigen expressed by a second cancer subject.

Embodiment P8. The method of any one of Embodiments P5-P8, wherein the standard control is an antigen expressed by a non-cancer subject.

Embodiment P9. The method of any one of Embodiments P5-P8, wherein the standard control is a cancer antigen expressed by a second cancer subject.

Embodiment P10. A method of producing an infectious disease antigen-binding antibody, the method comprising: (i) administering to an infectious disease subject an effective amount of an anti-CD73 antibody comprising a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3; (ii) isolating from the infectious disease subject a B cell expressing an infectious disease antigen-binding antibody; and (iii) expressing the gene encoding the infectious disease antigen-binding antibody, thereby producing an infectious disease antigen-binding antibody.

Embodiment P11. The method of Embodiment P10, wherein the isolating of step (ii) further comprises isolating a plurality of B cells from the infectious disease subject.

Embodiment P12. The method of Embodiment P11, further comprising isolating from the plurality of B cells a group of B cells expressing CD19, CD20, CD27 and/or CD38.

Embodiment P13. The method of Embodiment P12, further comprising isolating from the group of B cells a differentiated B cell expressing an infectious disease antigen-binding antibody.

Embodiment P14. The method of any one of Embodiments P10-P13, further comprising detecting a level of binding of the infectious disease antigen-binding antibody to an infectious disease antigen relative to a standard control.

Embodiment P15. The method of Embodiment P14, wherein the infectious disease antigen is an infectious disease antigen expressed by the infectious disease subject.

Embodiment P16. The method of Embodiment P14, wherein the infectious disease antigen is an infectious disease antigen expressed by a second infectious disease subject.

Embodiment P17. The method of any one of Embodiments P14-P16, wherein the standard control is an antigen expressed by a non-infectious disease subject.

Embodiment P18. The method of any one of Embodiments P14-P16, wherein the standard control is an infectious disease antigen expressed by a second infectious disease subject.

Embodiment P19. The method of any one of Embodiments P1-P18, wherein the B cell is a differentiated B cell.

Embodiment P20. The method of any one of Embodiments P1-P19, wherein the B cell expresses CD19, CD20, CD27 or CD38.

Embodiment P21. The method of any one of Embodiments P1-P20, wherein the B cell expresses CD19, CD20, CD27 and CD38.

Embodiment P22. The method of any one of Embodiments P1-P21, wherein the B cell is isolated from a blood sample, a lymphoid tissue sample or a bone marrow sample of the subject.

Embodiment P23. A method of treating cancer in a subject in need thereof, the method comprising, administering to the subject an effective amount of a cancer antigen-binding antibody formed by the method of any one of Embodiments P1-P9

Embodiment P24. The method of Embodiment P23, wherein the cancer is renal cancer, prostate cancer, lung cancer, melanoma, breast cancer, colorectal cancer, hepatocellular cancer, head and neck cancer or lymphoma.

Embodiment P25. A method of treating an infectious disease in a subject in need thereof, the method comprising administering to the subject an effective amount of an infectious disease antigen-binding antibody formed by the method of any one of Embodiments P10-P22.

Embodiment P26. The method of Embodiment P25, wherein the infectious disease is caused by Epstein Barr virus (EBV), Hepatitis C virus (HCV), Hepatitis B virus (HBV), Ebola virus, herpes simplex virus (HSV), cytomegalovirus (CMV), chikungunya virus, dengue virus, plasmodium or mycobacterium. The method of Embodiment P25, wherein the infectious disease is COVID-19, MERS, bronchiolitis, common cold, croup, influenza, penumonia, norovirus, rotavirus, adenovirus, astrovirus, measles, chicken pox, rubella, chickenpox, shingles, roseola, smallpox, fifth disease, chikungunya, hepatitis A, hepatitis B, hepatitis C, hepatitis D, hepatitis E, warts, genital warts, herpes simplex, genital herpes, molluscum contagiosum, Ebola, lassa fever, dengue fever, yellow fever, marburg hemorrhagic fever, Crimean-Congo hemorrhagic fever, polio, viral meningitis, viral encephalitis, rabies, a Zika virus, West Nile virus, cytomegalovirus malaria, or tuberculosis. The method of Embodiment P25, wherein the infectious disease is mumps, cytomegalovirus, rubella, measles, or herpes simplex-1.

Embodiment P27. A method of treating cancer in a subject in need thereof, the method comprising: (i) administering to a first cancer subject an effective amount of an anti-CD73 antibody comprising a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3; (ii) isolating from the first cancer subject a B cell expressing a cancer antigen-binding antibody; (iii) expressing the gene encoding the cancer antigen-binding antibody, thereby forming an isolated cancer antigen-binding antibody; and (iv) administering the isolated cancer antigen-binding antibody to a second cancer subject, thereby treating cancer in a subject.

Embodiment P28. The method of Embodiment P27, wherein the first cancer subject and the second cancer subject are the same.

Embodiment P29. The method of Embodiment P27, wherein the first subject and the second cancer subject are different.

Embodiment P30. The method of Embodiment P27, wherein the cancer is renal cancer, prostate cancer, lung cancer, melanoma, breast cancer, colorectal cancer, hepatocellular cancer, head and neck cancer or lymphoma.

Embodiment P31. A method of treating an infectious disease in a subject in need thereof, the method comprising: (i) administering to a first infectious disease subject an effective amount of an anti-CD73 antibody comprising a 1E9 antibody CDR L1, a 1E9 CDR L2, a 1E9 CDR L3, a 1E9 CDR H1, a 1E9 CDR H2, and a 1E9 CDR H3; (ii) isolating from the first infectious disease subject a B cell expressing an infectious disease-binding antibody; (iii) expressing the gene encoding the infectious disease-binding antibody, thereby forming an isolated infectious disease-binding antibody; and (iv) administering the isolated infectious disease-binding antibody to a second infectious disease subject, thereby treating an infectious disease in a subject.

Embodiment P32. The method of Embodiment P31, wherein the first infectious disease subject and the second infectious disease subject are the same.

Embodiment P33. The method of Embodiment P31, wherein the first infectious disease subject and the second infectious disease subject are different.

Embodiment P34. The method of Embodiment P31, wherein the infectious disease is caused by Epstein Barr virus (EBV), Hepatitis C virus (HCV), Hepatitis B virus (HBV), Ebola virus, herpes simplex virus (HSV), cytomegalovirus (CMV), chikungunya virus, dengue virus, plasmodium or mycobacterium. The method of Embodiment P31, wherein the infectious disease is COVID-19, MERS, bronchiolitis, common cold, croup, influenza, penumonia, norovirus, rotavirus, adenovirus, astrovirus, measles, chicken pox, rubella, chickenpox, shingles, roseola, smallpox, fifth disease, chikungunya, hepatitis A, hepatitis B, hepatitis C, hepatitis D, hepatitis E, warts, genital warts, herpes simplex, genital herpes, molluscum contagiosum, Ebola, lassa fever, dengue fever, yellow fever, marburg hemorrhagic fever, Crimean-Congo hemorrhagic fever, polio, viral meningitis, viral encephalitis, rabies, a Zika virus, malaria, or tuberculosis.

Embodiment P35. A method of detecting a cancer antigen, the method comprising: (i) contacting a cancer antigen with a cancer antigen-binding antibody formed by the method of any one of Embodiments P1-P9; and (ii) detecting binding of the cancer antigen-binding antibody to the cancer antigen, thereby detecting a cancer antigen.

Embodiment P36. A method of detecting an infectious disease antigen, the method comprising: (i) contacting an infectious disease antigen with an infectious disease antigen-binding antibody formed by the method of any one of Embodiments P10-P22; and (ii) detecting binding of the infectious disease antigen-binding antibody to the infectious disease antigen, thereby detecting an infectious disease antigen

Embodiment P37. The method of any one of Embodiments P1-P36, wherein the anti-CD73 antibody is administered at a half maximal effective concentration (EC₅₀) of at least 100 nM.

Embodiment P38. The method of any one of Embodiments P1-P37, wherein the anti-CD73 antibody is administered at an EC₅₀ of about 137 nM.

Embodiment P39. The method of any one of Embodiments P1-P37, wherein the anti-CD73 antibody is administered at an EC₅₀ of about 189 nM.

Embodiment P40. The method of any one of Embodiments P1-P39, wherein the effective amount is about 1 mg/kg, 3 mg/kg, 6 mg/kg, 10 mg/kg, 30 mg/kg, 40 mg/kg, or 120 mg/kg; or wherein the effective amount is from about 0.1 mg/kg to about 120 mg/kg.

Embodiment P41. The method of any one of Embodiments P1-P40, wherein the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO: 4, the CDR H2 has a sequence of SEQ ID NO:5, and the CDR H3 has a sequence of SEQ ID NO: 6.

Embodiment P42. The method of any one of Embodiments P1-P41, wherein the humanized heavy chain variable region comprises the sequence of SEQ ID NO:7.

Embodiment P43. The method of any one of Embodiments P1-P42, wherein the humanized light chain variable region comprises the sequence of SEQ ID NO:8.

Embodiment P44. The method of any one of Embodiments P1-P43, wherein the anti-CD73 antibody is an IgG.

Embodiment P45. The method of any one of Embodiments P1-P44, wherein the anti-CD73 antibody is an IgG1.

Embodiment P46. The method of any one of Embodiments P1-P44, wherein the anti-CD73 antibody is an IgG4.

Embodiment P47. The method of any one of Embodiments P1-P46, wherein the anti-CD73 antibody is a Fab′ fragment.

Embodiment P48. The method of any one of Embodiments P1-P46, wherein the anti-CD73 antibody is a single chain antibody (scFv).

Embodiment P49. The method of any one of Embodiments P1-P48, wherein the anti-CD73 antibody is capable of binding a CD73 antigen with an equilibrium dissociation constant (K_{D}) from about 0.3 to about 25 nM.

Embodiment P50. The method of any one of Embodiments P1-P49, wherein the anti-CD73 antibody is capable of binding a CD73 antigen with an equilibrium dissociation constant (K_{D}) of about 0.64 nM.

Embodiment P51. The method of any one of Embodiments P1-P50, wherein the anti-CD73 antibody is capable of binding a CD73 antigen at a pH of less than about 7.5.

Embodiment P52. The method of any one of Embodiments P1-P51, wherein the anti anti-CD73 antibody body is capable of binding a CD73 antigen at a pH from about 6.0 to about 7.0.

Embodiment P53. The method of any one of Embodiments P1-P52, wherein the anti-CD73 antibody is capable of binding a CD73 antigen at a pH of about 6.3.

Embodiment P54. A pharmaceutical composition comprising an effective amount of an anti-CD73 antibody comprising a 1E9 CDR L1, a 1E9 CDR L2, a 1E9 CDR L3, a 1E9 CDR H1, a 1E9 CDR H2, and a 1E9 CDR H3, an adjuvant and a pharmaceutical excipient.

Embodiment P55. The pharmaceutical composition of Embodiment P54, wherein the adjuvant is a neoantigen peptide.

Embodiment P56. The pharmaceutical composition of Embodiment P54, wherein the adjuvant is an influenza virus or a functional fragment thereof.

Embodiment P57. The pharmaceutical composition of Embodiment P54, wherein the adjuvant is SARS-CoV or a functional fragment thereof.

### EXAMPLES

The following examples are for purpose of illustration only and are not intended to limit the spirit or scope of the disclosure or claims.

### Example 1

B cells are isolated from peripheral blood or resected tissue specimen before and/or after CPI-006 treatment. B cells are isolated by labeling unwanted cells with antibody complexes and magnetic particles (negative selection, e.g., Stem Cell Technologies EasySep Human B Cell Isolation Kit) or through positive selection using fluorescence-activated cell sorting (FACS). Fluorochrome conjugated human antibody cocktails to identify B cells include anti-human IgG, anti-human IgM, anti-human IgD, anti-human CD38, anti-human CD138, anti-human CD19, anti-human CD20, anti-human CD4, anti-human CD71. In all cases, B cell populations are enriched using a cocktail of antibodies to exclude granulocyte, monocyte/macrophage, dendritic, NK, and CD8 cell populations. Specifically, mature B cells that had undergone class switching (e.g., CD38Hi, IgGPos, IgDLow, CD71Hi) are the target cell type to be isolated. Single cell sequencing is performed to determine and pair heavy and light chain immunoglobulin (Ig) sequences from individual B cells. Heavy and light chain sequences or complementarity-determining regions (CDRs) are cloned or synthesized and cloned into mammalian expression vector. In embodiments, antibody sequences that are enriched or novel after CPI-006 treatment are cloned or synthesized. This approach helps to screen out antibodies already present in the pre-treatment samples and therefore not a candidate anti-tumor antibody induced by CPI-006. https://www.genscript.com/transient-vs-stable-expression.html

Examples of expression vectors are pCEP4, (http://wolfson.huji.ac.il/purification/PDF/Literature/VazquezLombardi2017.pdf) or pFUSE/pTRIOZ (https://www.invivogen.com/antibody-generation) for transient or stable production of antibodies. Mammalian cell lines (e.g., CHO, HEK293) are transfected with expression vectors encoding antibody heavy and light chains. The culture supernatant is collected after culturing for days or weeks. The recombinant antibody is purified from culture supernatant using Protein A/G, for example. Recombinant antibodies are screened for binding to tumor associated antigens. For example, a human cancer cell is used that a candidate antibody binds to. The target antigen is then be identified by: Immunoprecipitation followed by mass spectrometry (either LC-MS or LC-MS/MS, see https://www.ncbi.nlm.nih.gov/pmc/articles/PMC5424237/); CRISPR based genetic screen; genes are randomly deleted from the genome, cells that the antibody can no longer bind to are identified. NGS is used to determine which gene was deleted by CRISPR, the gene will be the target of the candidate antibody. Approach 1: The human cell can be a human cancer cell line, a primary cancer cell (from fresh tumor suspension or formalin fixed paraffin embedded tissue specimen), a cancer organoid culture, or human cell perpetuated as a xenograft in immunocompromised mice. Approach 2: Whole exome sequencing is performed on tumor cells isolated from autologous tissue specimen collected from a patient. Recombinant mutant proteins/peptides predicted by any of the multitude of computer algorithms that currently exist are identified and synthesized. Then test if candidate antibody binds to mutant proteins/peptides using ELISA, flow cytometry, or other appropriate methods known in the art.

### Example 2

### CPI-006 Inhibits CD73 Enzyme Function and Reverses Adenosine-Mediated Immunosuppression

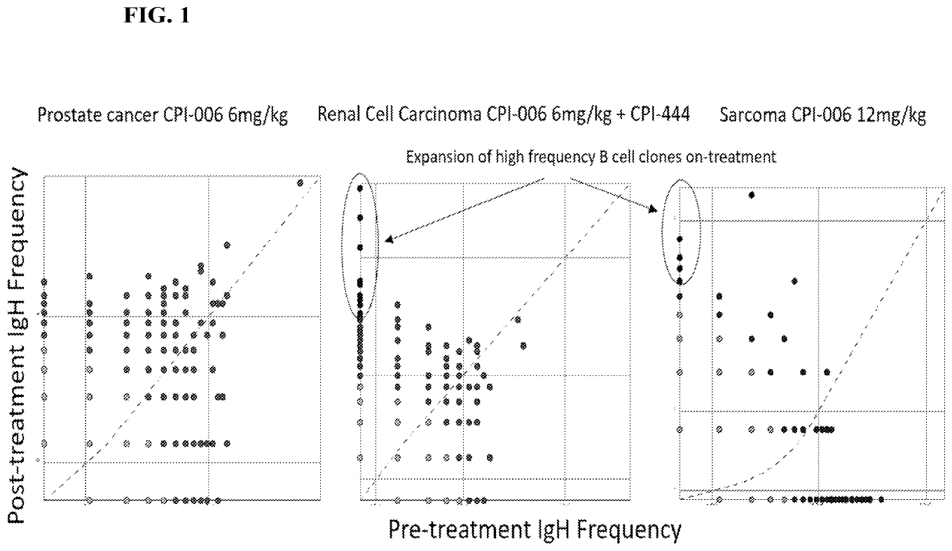

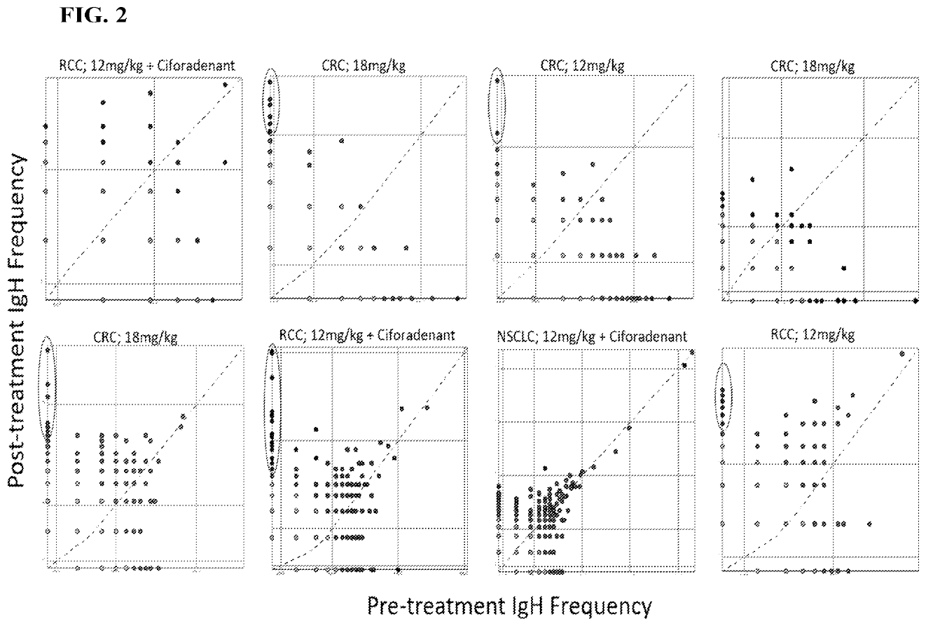

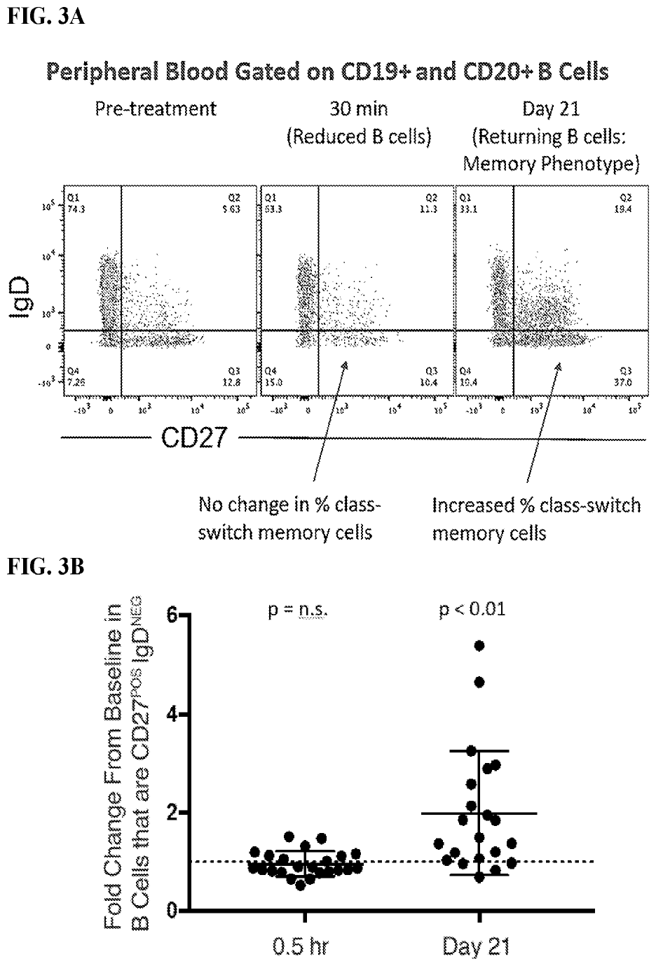

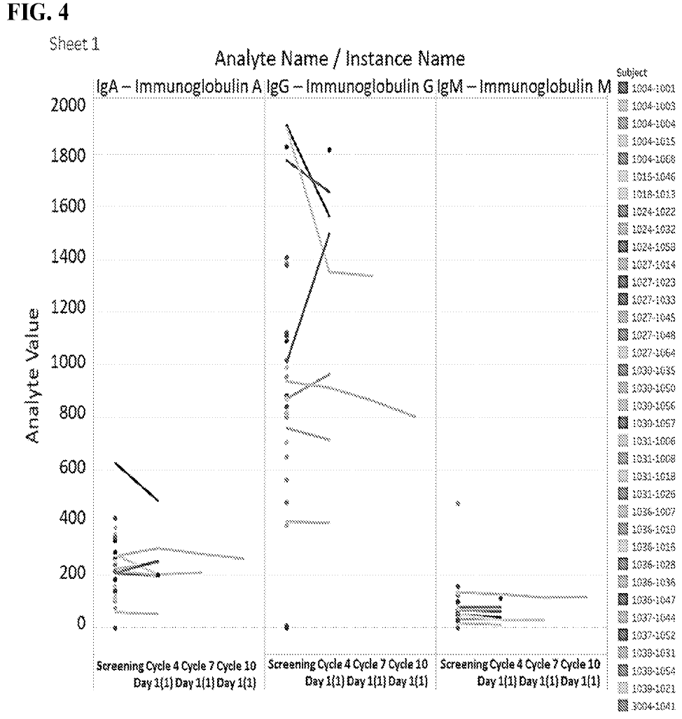

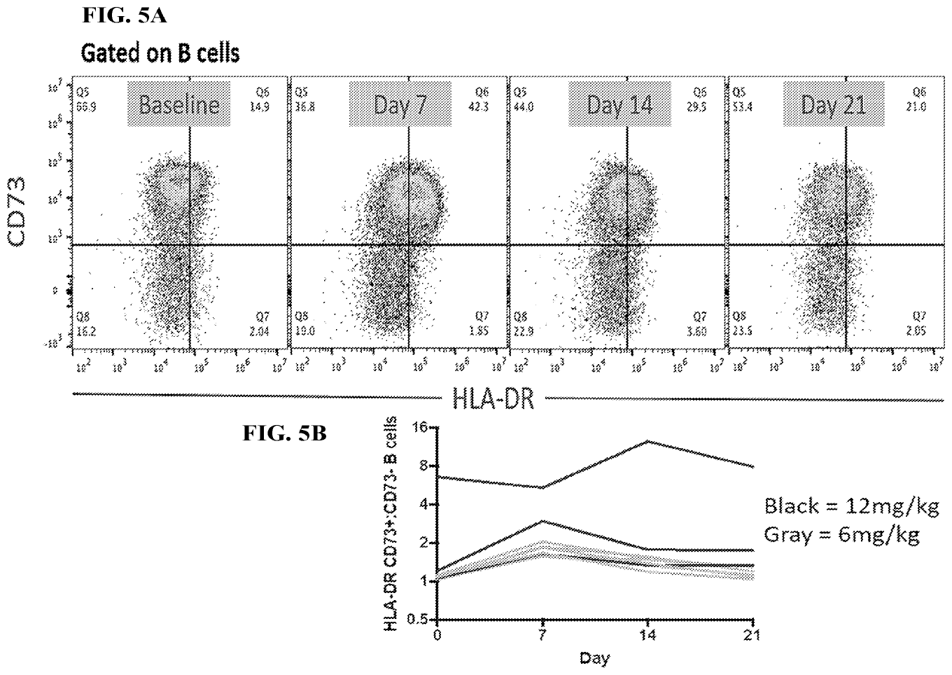

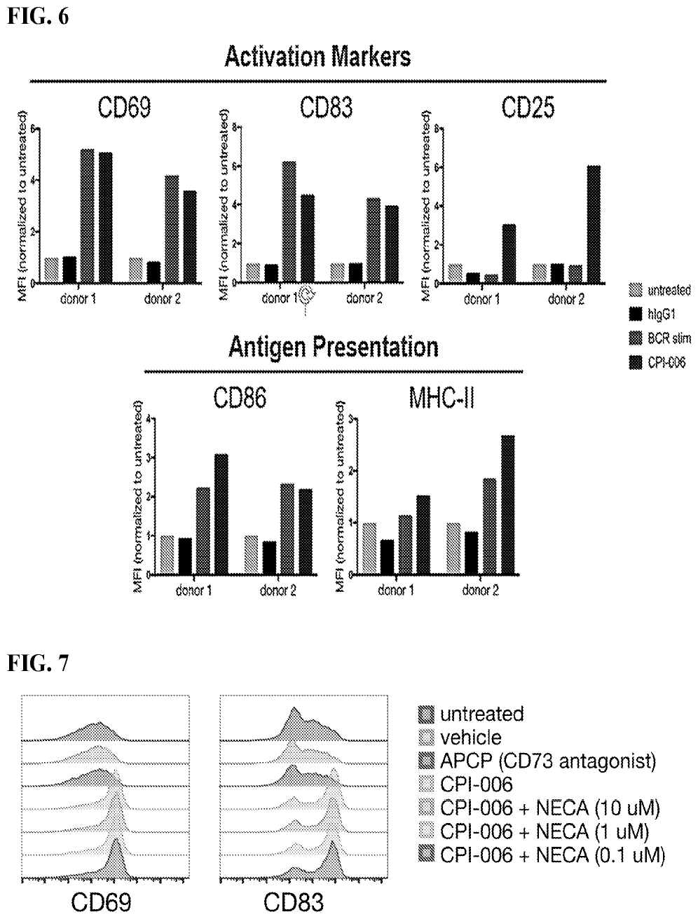

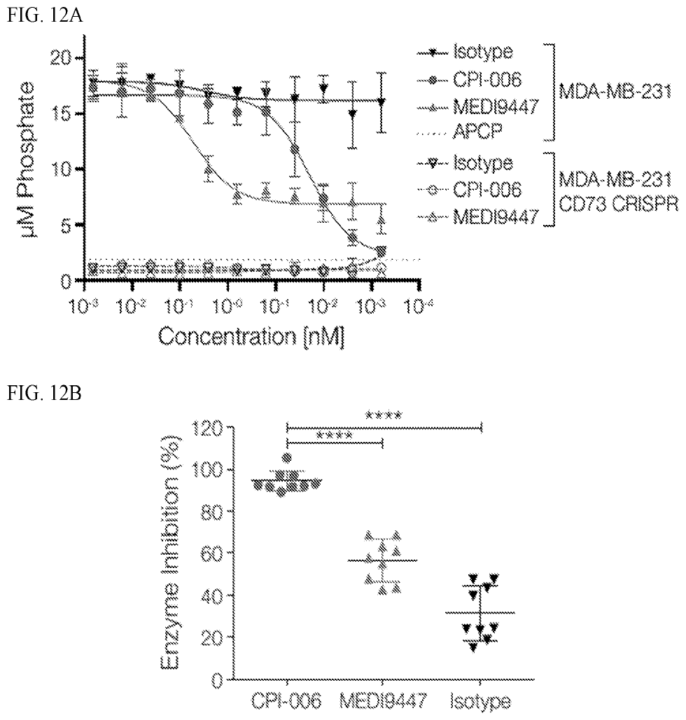

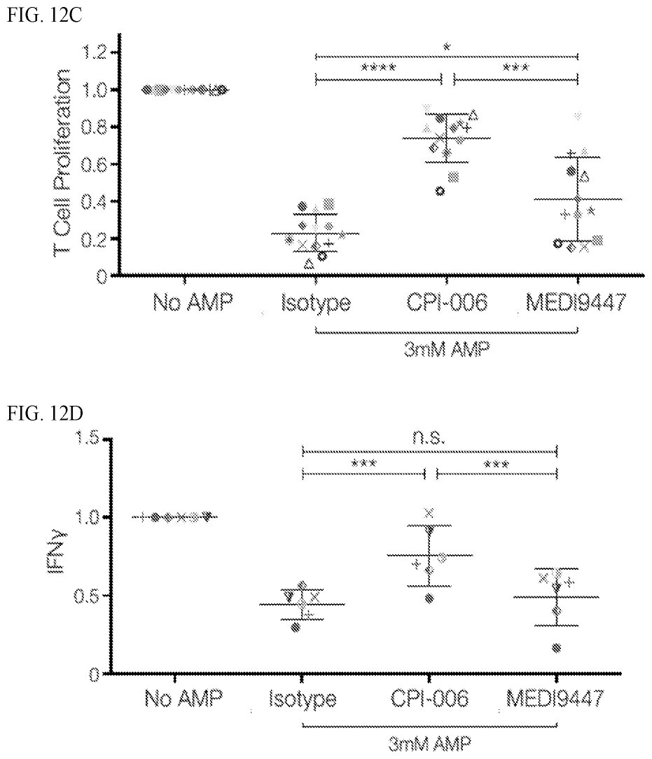

CPI-006 is a humanized anti-CD73 IgG1k antibody engineered with an N297Q mutation in CH2 of the heavy chain to eliminate Fc effector functions such as the ability to fix complement and initiate antibody dependent cellular cytotoxicity (ADCC, Supplemental FIG. 1A in Willingham et al, medRxiv, Sep. 15, 2020 at https://www.medrxiv.org/content/10.1101/2020.09.10.20191486v1). CPI-006 binds human CD73 with a KD of 200 pM measured using bio-layer interferometry (Supplemental FIG. 1B in Willingham et al, medRxiv, Sep. 15, 2020). The ability of this antibody to inhibit the enzymatic activity of cellular CD73 was evaluated by directly measuring free phosphate levels generated upon conversion of AMP to adenosine. CPI-006 inhibited CD73 activity to baseline levels that were established using the CRISPR CD73 knockout cell line and comparison to treatment with saturating amounts of APCP (adenosine 5′-(a,b-methylene) diphosphate), a small molecule inhibitor of CD73 enzymatic activity (FIG. 12A). In contrast, oleclumab, another anti-CD73 antibody that binds a non-overlapping epitope, only partially reduced CD73 activity. [19] CPI-006 also eliminated the enzymatic activity of CD73 expressed on primary human peripheral blood mononuclear cells (PBMCs) while oleclumab demonstrated a partial effect on enzymatic activity (FIG. 12B). Collectively, these data show that CPI-006 and oleclumab inhibit catalytic activity of cellular CD73 expressed by tumor cells and lymphocytes, but only CPI-006 completely abolishes CD73 enzymatic activity.

To model the immunosuppressive effects of adenosine on T cells, human PBMCs were obtained from healthy donors and cultured under T cell activating conditions. Addition of AMP resulted in decreased T cell proliferation and cytokine secretion. Addition of CPI-006 restored T cell proliferation in all donors tested, consistent with the antibody preventing conversion of AMP into immunosuppressive adenosine (FIG. 12C). Oleclumab restored T cell proliferation in a subset of donors but did not reach the magnitude of the CPI-006 response (FIG. 12C). Interferon-gamma (IFNg) production was evaluated as an additional readout for T cell response and similar results were observed (FIG. 12D). These data demonstrate that antibody-mediated inhibition of CD73 activity blocks the suppressive effects of adenosine on T cell proliferation and cytokine secretion.

### Example 3

### CPI-006 Directly Activates Human B Cells In Vitro

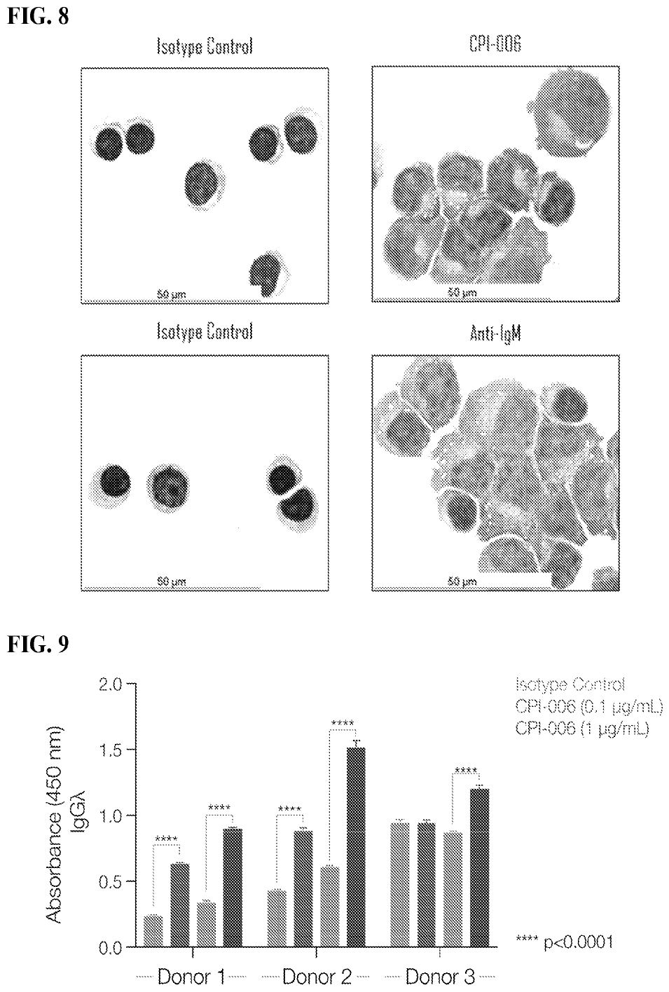

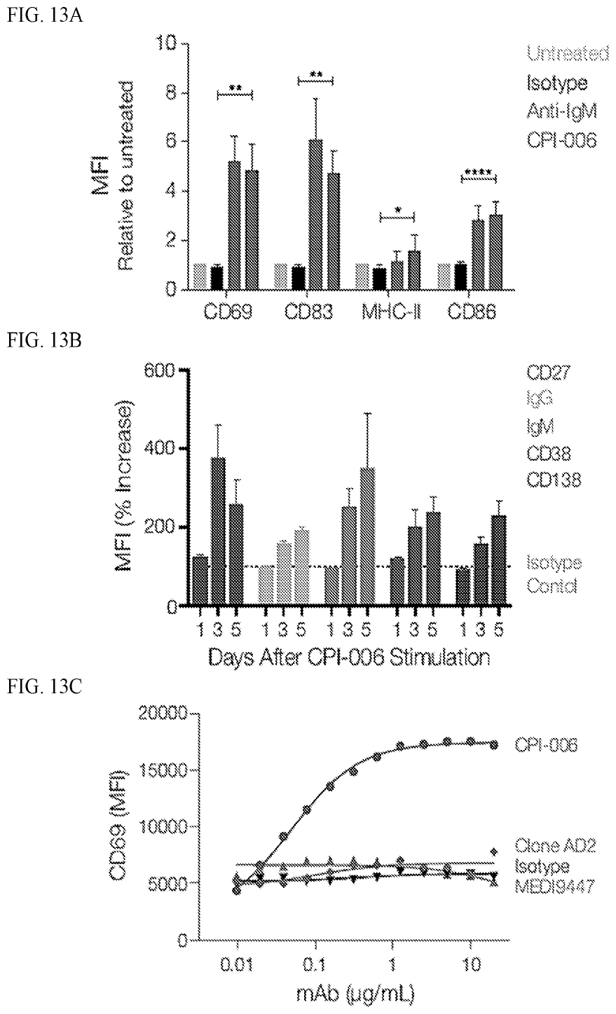

CD73 is expressed on subsets of human hematopoietic cells and had previously been implicated to play a role in lymphocyte activation and adhesion [12, 13, 20, 21]. The inventors performed a flow cytometry-based screen to identify differentially expressed cell surface markers on immune cells following in vitro treatment with CPI-006. CPI-006 strikingly activated B lymphocytes, resulting in the upregulation of activation markers (CD69 and CD83) and antigen presentation machinery (CD86 and MHC-II) to similar levels achieved with the positive control of BCR crosslinking via anti-IgM (FIG. 13A). CPI-006 mediated B cell activation also resulted in increased cell surface expression of CD27, IgG, CD38, and CD138, all markers consistent with induction of B cell maturation (FIG. 13B). CPI-006 induced activation was dose-dependent with concentrations of 1 μg/mL achieving near maximal induction of CD69 in vitro (FIG. 13C). B cell activation has not previously been described in relation to CD73 signaling and is seemingly unique to CPI-006, as both oleclumab and clone AD2, two anti-CD73 antibodies that do not cross-block CPI-006, fail to induce activation (FIG. 2C). Naïve B cells stimulated with CPI-006 in vitro underwent morphological changes consistent with differentiation into plasmablasts (FIG. 8). CPI-006 mediated B cell activation was restricted to CD73^{POS} B cells and required bivalent binding as CPI-006 immunoglobulin Fab fragments had minimal effect on expression levels of CD69 (FIG. 2D). CPI-006 induction of CD69 was blocked with ibrutinib, a covalent BTK inhibitor (FIG. 2E), demonstrating that CPI-006 directly activates B lymphocytes by invoking canonical B cell signaling pathways. B cell activation was not a consequence of the concentration of extracellular adenosine as addition of NECA (5′-(N-ethylcarboxamido)adenosine), a potent and stable analogue of adenosine, did not block the induction of activation markers by CPI-006 (FIG. 7).[22]

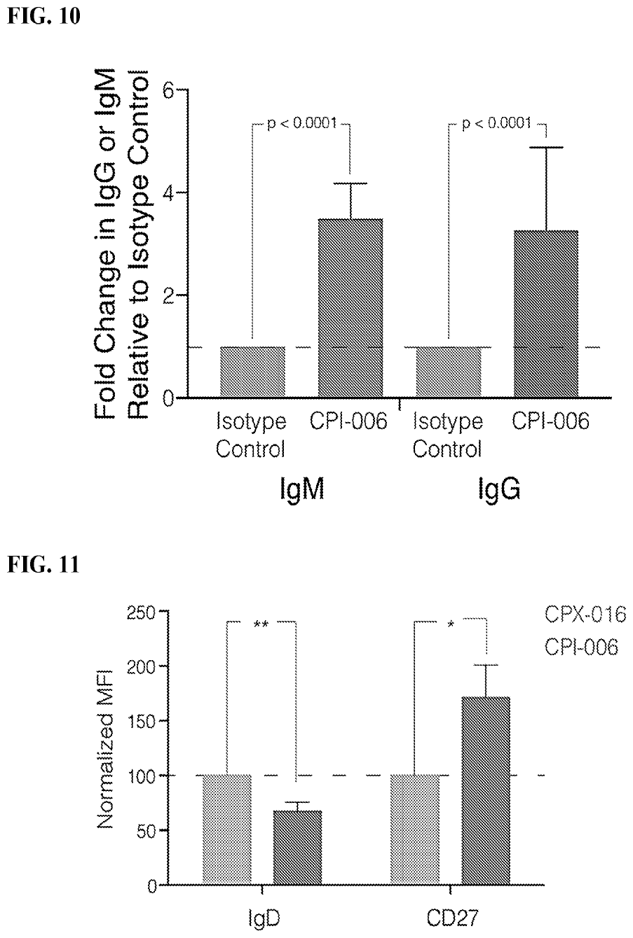

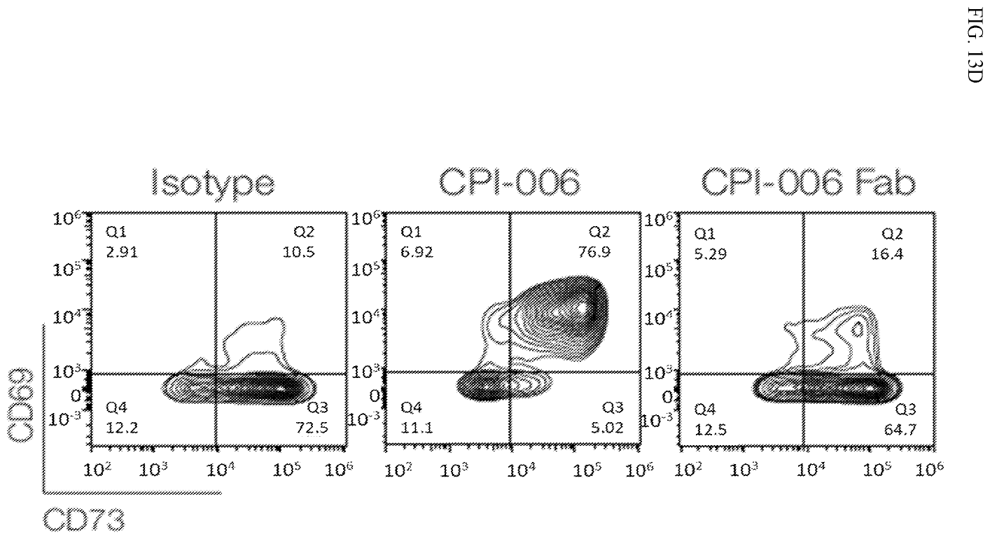

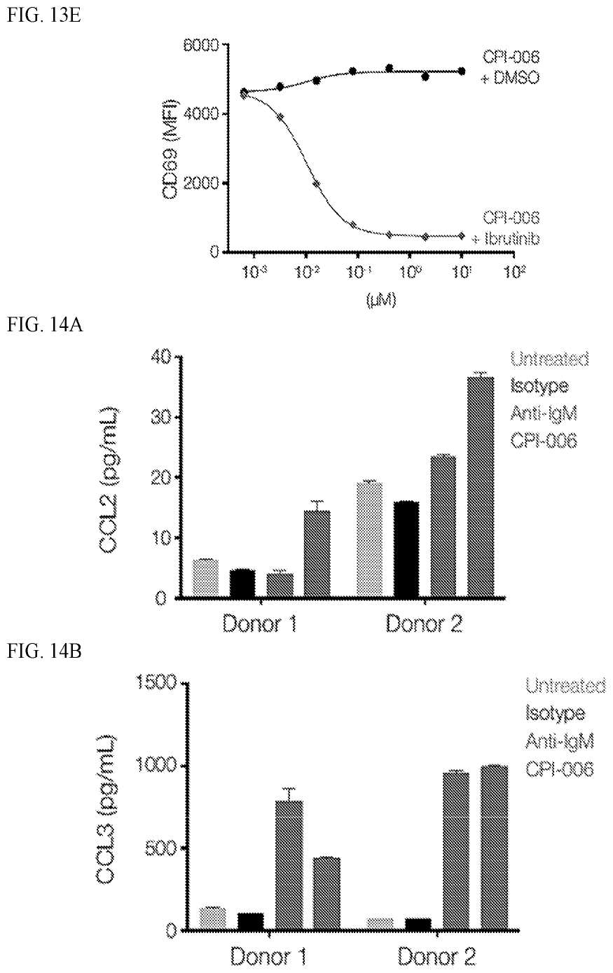

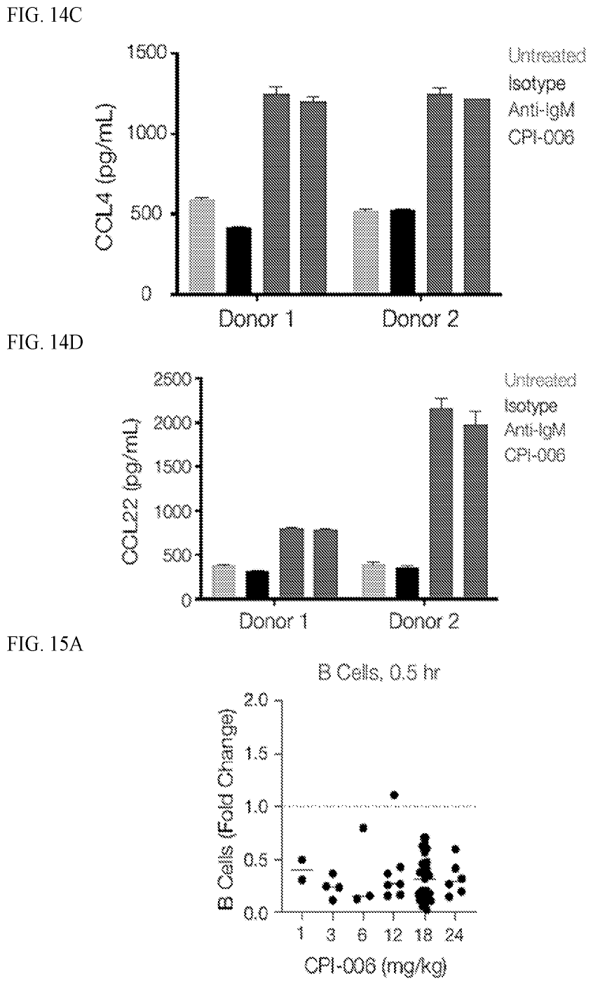

To assess functional consequences of B cell activation with CPI-006, the inventors first measured the concentration of IgG and IgM secreted by healthy donor PBMCs into the culture supernatant six days after in vitro treatment. Addition of CPI-006 resulted in a 3-fold increase in IgM and IgG1 levels (IgGk not measured) relative to an isotype control, demonstrating that CPI-006 stimulates antibody secretion and possibly class switching to IgG (FIG. 10). CPI-006 also induced production of B cell cytokines such as CCL3, CCL4, CCL2, and CCL22 (FIGS. 14A-14D), but did not induce IFNg, IL-2, IL-6, IL-10, or TNFa from healthy donor PBMCs (data not shown). CPI-006 would therefore not be expected to increase the potential for inflammatory cytokine release reported in some COVID-19 patients. [23-25] Collectively, these experiments demonstrated that CPI-006 activated B lymphocytes, resulting in morphological and immunological changes consistent with B cell differentiation and antibody production.

Furthermore, this property is unique to CPI-006 and is independent of adenosine-modulatory activity.

### Example 4

### Immunologic Effects of CPI-006 in a Phase 1 Trial in Cancer Patients

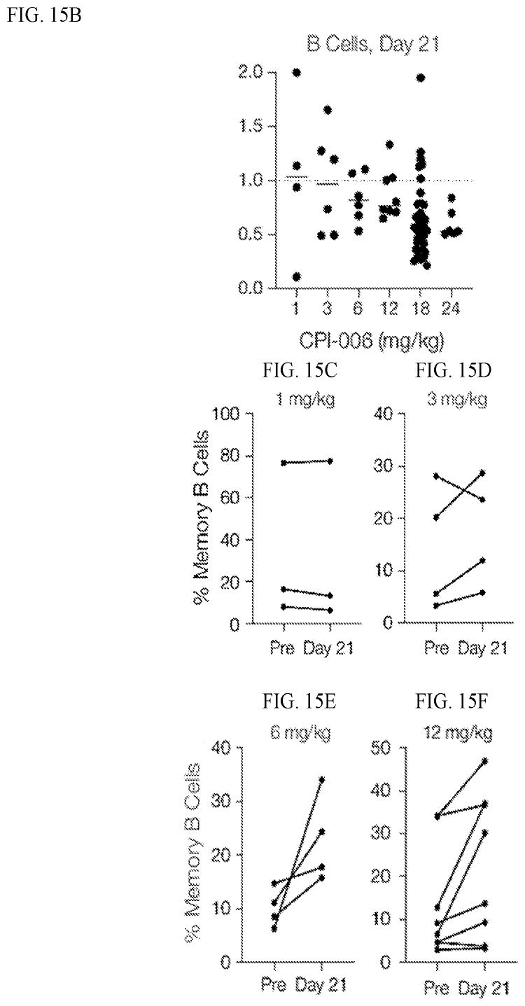

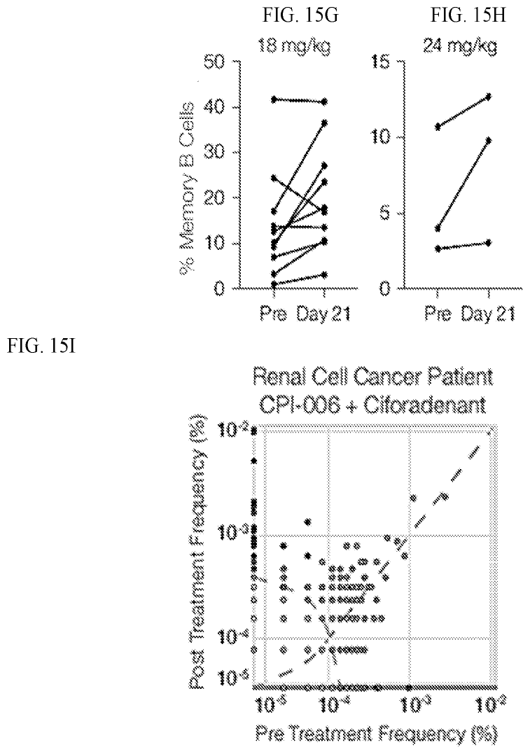

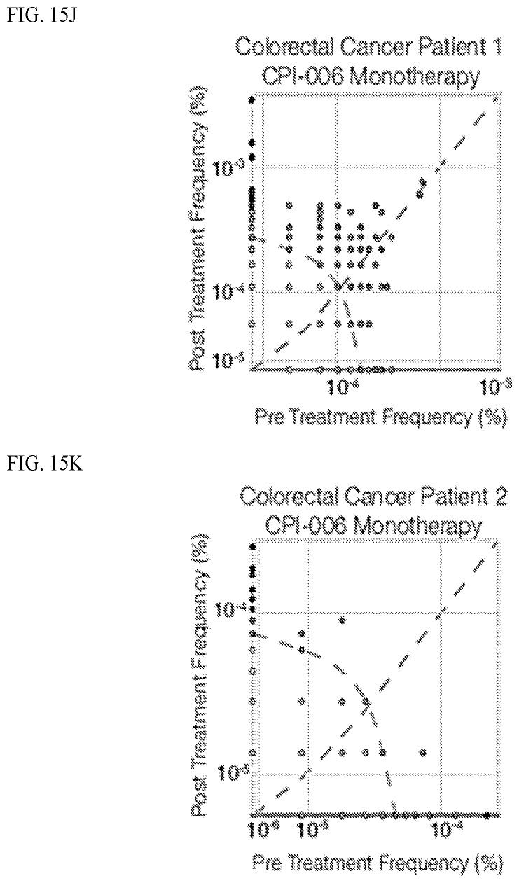

CPI-006 is being evaluated as an immunotherapy for cancer in an ongoing phase 1 study (NCT03454451). The cancers being evaluated in the study include non-small cell lung cancer, renal cell cancer, colorectal cancer, triple negative breast cancer, cervical cancer, ovarian cancer, pancreatic cancer, endometrial cancer, sarcoma, squamous cell carcinoma of the head and neck, bladder cancer, metastatic castration resistant prostate cancer, and non-Hodgkin lymphoma. In this dose escalation, repeat dose (21-day cycle) study, the inventors observed dramatic decreases in circulating CD73^{POS} B cells at all CPI-006 dose levels (1-24 mg/kg), 30 minutes after antibody infusion (FIGS. 15A-15B). CPI-006 does not induce B cell death or initiate ADCC (Supplemental FIG. 1A in Willingham et al, medRxiv, Sep. 15, 2020 at https://www.medrxiv.org/content/10.1101/2020.09.10.20191486v1) so this result is due to the redistribution of activated B cells into lymphoid tissues. B cells returned to circulation at levels similar to baseline by day 21. Returning B cells were enriched in CD27^{POS} IgD^{NEG} class switched memory B cells in the majority of subjects receiving ≥3 mg/kg CPI-006 (FIGS. 15C-15H). Memory B cells are essential for both acute and long-term immunity as they have undergone immunoglobulin rearrangement and somatic hypermutation in order to produce high affinity, antigen-specific antibody upon exposure to antigen. BCR repertoire analysis in a subset of patients revealed the emergence of novel B cell clones that were not present in the peripheral blood prior to treatment (FIG. 15I-15K). These new B cell clones were limited to 2-40 clones per patient and appeared with frequencies as high as 1:100 B cells, consistent with a robust clonal expansion following an antigen-specific response (FIG. 15I-15K). The cognate antigens recognized by these new B cell clones are currently under characterization. Representative CPI-006 treated patients (n=10) with robust diversification of BCR repertoire demonstrate unaltered antigen-specific IgG response against 5 common viruses, including mumps, cytomegalovirus (CMV), rubella, measles, and herpes simplex virus-1 (HSV1), at 21 days post treatment compared to baseline. This result demonstrated that CPI-006 does not simply induce polyclonal B cell activation and shows a requirement for concomitant antigen exposure in order for CPI-006 to induce an antigen-specific response (Supplemental FIG. 2 in Willingham et al, medRxiv, Sep. 15, 2020).

### Example 5

A randomized control study will be conducted to evaluate the improvement in anti-SARS CoV-2 antibody production and clinical benefit in patients with mild to moderately severe COVID-19 treated with CPI-006. The study will demonstrate that CPI-006 will enhance humoral immune response to SARS-CoV-2; will provide long-term immunity to SARS-CoV-2 (and related SARS viruses); and will produce neutralizing antibodies SARS-CoV-2 (and related SARS viruses), and will shorten the duration of COVID-19 and improve clinical outcomes. The results will provide evidence that CPI-006 will be useful for other SARS viruses (e.g., SARS-CoV).

This Phase 1b/2 study is an open label, prospective, randomized trial evaluating CPI-006 as immunotherapy for mild or moderately symptomatic COVID-19 patients. Randomization will be 2:1 with experimental and control patients both receiving the standard of care. COVID-19 disease will be confirmed by reverse transcriptase polymerase chain reaction (PCR) testing of nasal swabs. There will be no placebo administered to the control group. Patients may be enrolled who are managed as inpatients in the hospital or as outpatients and must have blood oxygen saturation of at least 94% on room air. Patients meeting study entry criteria will receive a single 10-30 minute intravenous infusion of 200 mg of CPI-006 given on Day 1 of study. Control group will not receive treatment. Treatment and control groups will be followed up to six months for production of anti-SARS-CoV-2 antibodies. Safety and other disease assessments will be conducted at 14 and 28 days and at 2-3 month intervals thereafter for up to six months.

Study Assessments. Day 14, 28, 56, 84, 160 serum antibody titer to SARS-CoV-2 virus. The assays will measure IgM and IgG using ELISA method. Antibodies to the viral spike protein will be quantitated. Antibody response to the spike protein receptor binding domain (RBD) also will be measured. Other disease assessments include Day 14 nasal swab PCR test, time to requiring hospitalization, or time to discharge and oxygen saturation. Safety to changes from baseline in labs, physical exam will be assessed.

Serum at baseline and at specified times will be stored for use in various serology studies which include: anti-SARS-CoV-2 receptor binding domain antibodies; anti-SARS-CoV-2 antibodies binding to total spike protein; neutralizing antibodies that block viral infection in vitro; and cross-reactive antibodies to other coronaviruses. Peripheral blood will be evaluated for treatment effects on peripheral blood mononuclear cells by immunophenotyping using flow cytometry. B cells will be isolated and analyzed for expression of BCR in order to determine clonal distribution of B cells and changes with treatment.

The primary efficacy endpoint is the titer of anti-SARS-CoV-2 antibody response at Day 28 based on the Efficacy Evaluable Population. The change of the titer of anti-SARS-CoV-2 from baseline of each participant will be calculated. The two treatment arms will be compared by the change of anti-SARS-CoV-2 antibody from baseline. The antibody titer of serum collected from trial subjects will be measured using an ELISA assay. The method is based on reactivity to the immunogenic spike protein of the virus. A recombinant form of the spike protein will be absorbed to the surface of a 96-well microtiter plate. A serial dilution of patient serum will be dispensed to the wells and will include a negative control to establish the baseline value. Antibodies recognizing the viral antigen will be allowed to react before removal of unbound immunoglobulin from the wells. Anti-SARS-CoV-2 IgG and IgM bound to the spike protein will be detected using standard ELISA techniques. The assay typically provides a sigmoidal curve of positive antibody reactivity that diminishes as a function of the dilution. The endpoint titer for each subject will be equal to the highest dilution of serum that yields a value above the baseline provided by the control.

All the secondary efficacy endpoints will be analyzed based on the Efficacy Evaluable population. Viral PCR test by nasal swab at Day 14: proportion of participants with nasal swab PCR negative by day 14 will be compared between the two treatment arms. Area under the titer of anti-SARS CoV 2 antibody response curve: the area under the titer of anti-SARS-CoV-2 antibody response curve versus the control at days 14, 28, 56, 84 and 160 will be calculated by the trapezoidal rule, and the area under the curve will be compared between the two arms. For outpatients, the proportion of participants and their time to hospitalization will be compared between the two treatment arms. For in patients, the proportion of participants and their time to discharge from the hospital will be compared between the two treatment arms. Safety analyses will be performed based on the Safety Population.

The study results will demonstrate that CPI-006 meets the primary and secondary endpoints described herein, such that CPI-006 will successfully treat COVID-19. CPI-006 will improve clinical outcomes, such as reducing the need for hospitalization, reducing the duration of hospitalization, reducing the need for a ventilator and/or admission to ICU, and reducing the incidence of death.

### Example 6

CPI-006-002 is a Phase 1 open label, dose-escalation trial evaluating the safety and immunologic effects of a single dose of CPI-006 as immunotherapy for hospitalized patients with mild to moderately severe COVID-19 (NCT04464395). SARS-CoV-2 infection was confirmed by RT-qPCR testing of nasal swabs and eligible patients had blood oxygen saturation of at least 92% on ≤5 L/min supplemental oxygen. Five patients per cohort received doses of 0.3 mg/kg, 1.0 mg/kg, 3.0 mg/kg or 5.0 mg/kg delivered by intravenous infusion over 10-30 minutes. Patients were allowed to receive standard care for COVID-19, including remdesivir and steroids. Use of CCP or passively administered monoclonal anti-SARS-CoV-2 antibodies were not permitted. Safety and other disease assessments along with PBMC and serum collection were/will be conducted at 7, 14, and 28 days, and at 2 months, 3 months and 6 months after receiving CPI-006.

### Patient Characteristics and Safety

Five patients have been treated in the 0.3 mg/kg and five in the 1.0 mg/kg cohorts (Table 1). The median age was 64 years (range 28-76) and all had comorbidities including diabetes (4), hypertension (2), obesity (7, and/or cancer (2). The median duration from presentation of symptoms (POS) to CPI-006 administration was 8 days (range 1-21 days). No infusion related reactions or other treatment related adverse events have been observed. All patients recovered with improvement of inflammatory markers and symptoms and were discharged at a median of 4 days after hospitalization.

TABLE 1

| | | Onset of Symptoms | | | |
|---|---|---|---|---|---|
| CPI-006 | Age, Gender, | until CPI-006 | | Baseline | Day |
| Dose | Race, BMI | Treatment (Days) | Comorbidities | ALC K/mm³ | Discharged |
| 0.3 | 70-80; M; W; | 1 | Diabetes, CAD, | 0.9 | 4 |
| | 32.1 | | HTN | | |
| 0.3 | 20-30; M; AA; | 4 | Diabetes | 1.5 | 6 |
| | 29.9 | | | | |
| 0.3 | 40-50; M; AA; | 8 | Asthma | 1.3 | 4 |
| | 32.3 | | | | |
| 0.3 | 60-70; F; AA; | 4 | Breast cancer, | 1.3 | 5 |
| | 30.3 | | HTN | | |
| 0.3 | 20-30; F; AA; | 5 | Asthma | 0.8 | 4 |
| | 33.7 | | | | |
| 1.0 | 60-70; M; AA; | 15 | HTN, CAD, | 1.1 | 7 |
| | 16.5 | | COPD | | |
| 1.0 | 70-80; F; AA; | 4 | Diabetes, HTN | 0.6 | 6 |
| | 35.1 | | | | |
| 1.0 | 60-70; M; A; | 8 | BPH | 0.9 | 4 |
| | 28.9 | | | | |
| 1.0 | 60-70; M; H; | >21 | Sleep apnea, HTN, | 0.9 | 6 |
| | 33.2 | | diabetes | | |
| 1.0 | 30-40; F; O; | 3 | — | 1.2 | 3 |
| | 32.1 | | | | |

In Table 1, “F” is female; “M” is male; “W” is white; “AA” is African American; “A” is Asian; “H” is Hispanic; “O” is other; “CAD” is coronary artery disease; “HTN” is hypertension; “COPD” is chronic obstructive pulmonary disease; “BPH” is benign prostatic hyperplasia.

### Immune Responses in CPI-006 Treated Patients

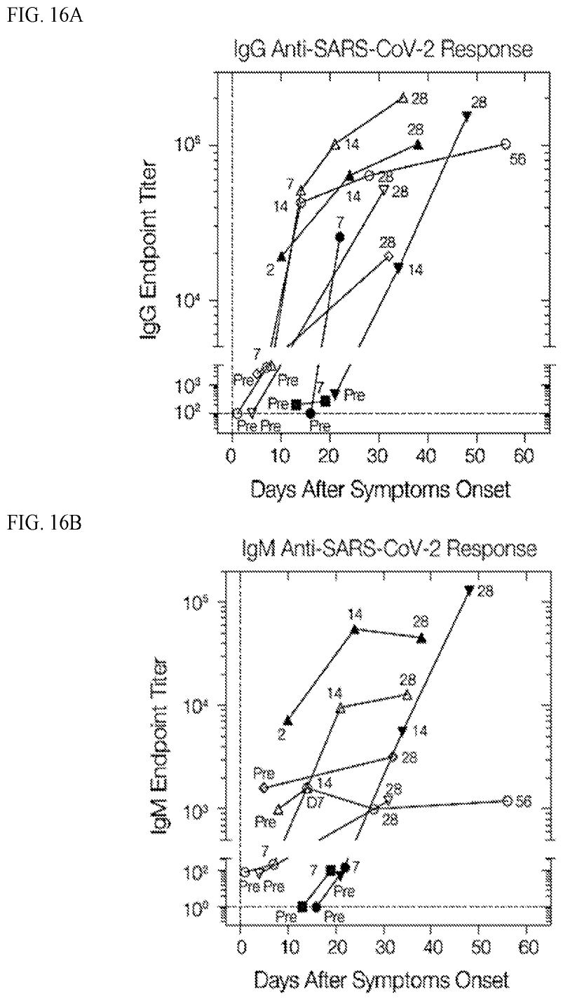

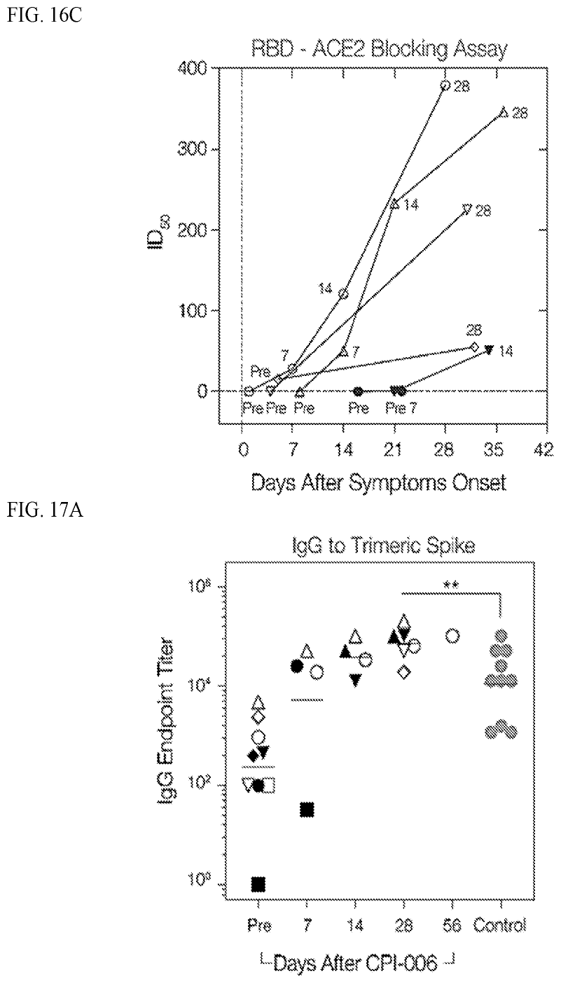

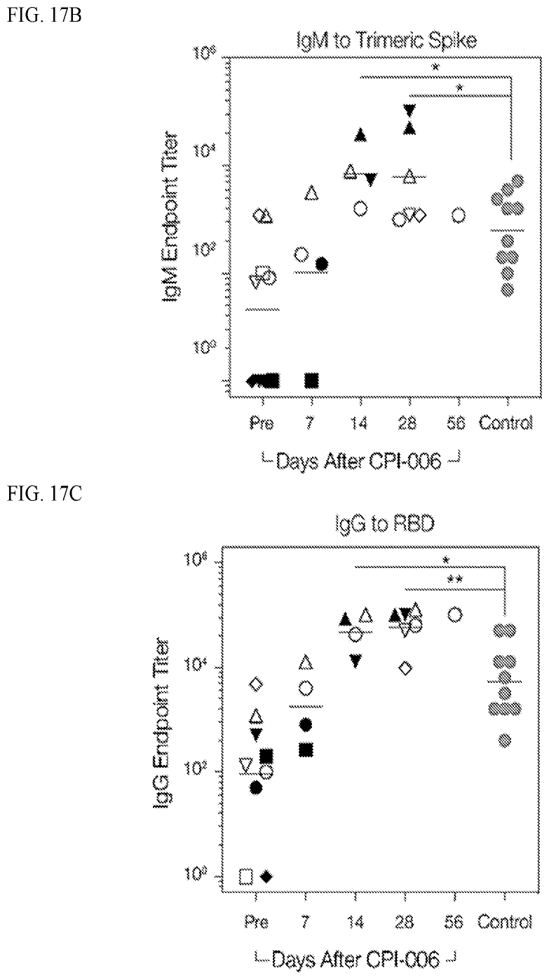

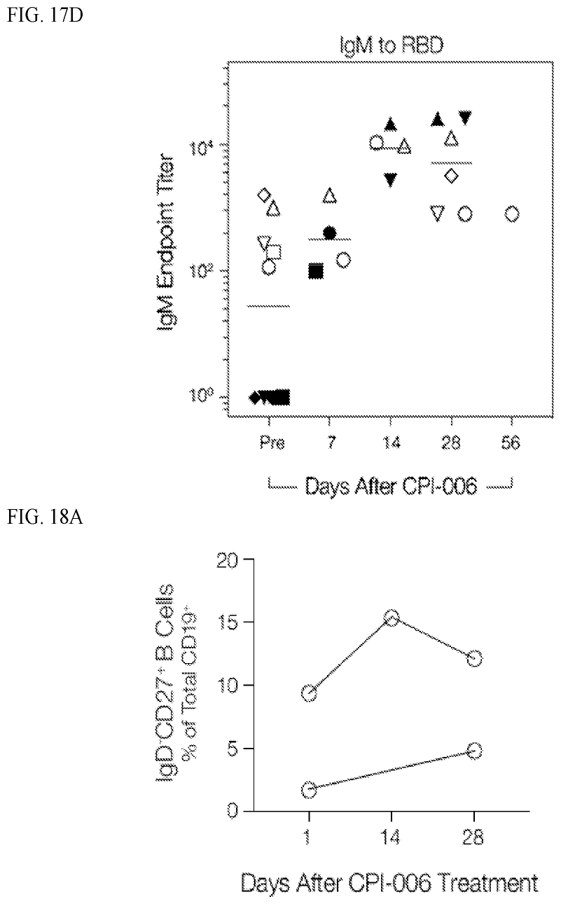

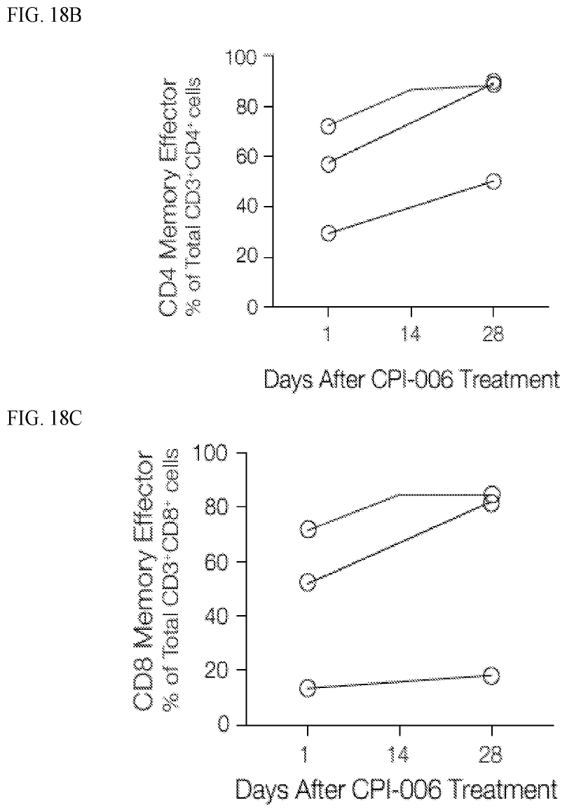

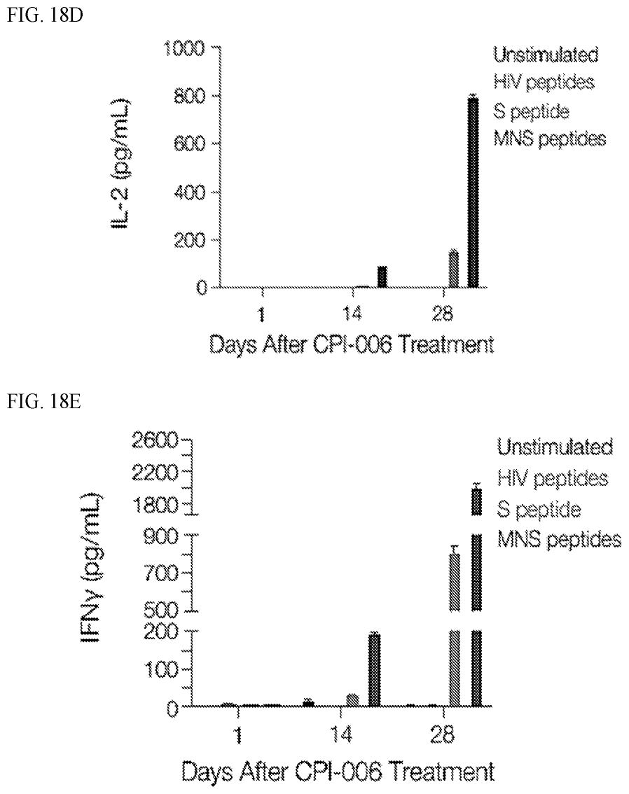

IgG and IgM antibody titers against the SARS-CoV-2 TS and/or RBD rapidly increased in 8/8 evaluable patients within 7 days of a single infusion of low doses of CPI-006 (FIGS. 16A-16B). One patient did not have a pre-treatment sample available, but had a sample collected 24 hours after CPI-006 administration and this sample exhibited a high antibody titer. Similar results were observed in anti-SARS-CoV-2 IgA titers (data not shown). No correlation between time after POS and pre-treatment serum antibody levels has been observed as 9/9 evaluable patients had low pre-treatment titers despite relatively long durations of symptoms. For example, one patient had low titers at 21 days after onset of symptoms, but produced IgG and IgM titers rapidly rising to >100,000 and extending 28 days after administration of CPI-006, equivalent to 49 days POS. A corresponding increase in neutralizing antibody levels, measured by the ability to block recombinant RBD binding to human ACE2, was also observed (FIG. 16C). Results in this assay have been shown by others to correlate with results in the live viral plaque reduction neutralization assay test (PRNTs).[26] Continually increasing IgG and IgM levels to viral TS or RBD antigens and neutralization antibodies were observed out to 28 days post treatment. Prolonged high titer humoral response is possible as one patient has escalating levels out to 56 days. Antibody levels reached higher titers compared to convalescent sera from recovered patients with more favorable clinical characteristics (convalescent sera collected at 4-6 weeks POS, no comorbidities, and median age of 40) (FIGS. 17A-17C). Immunophenotyping of PBMCs at baseline and 14- or 28-days after treatment provided evidence that CPI-006 increased the frequency of memory B cells in two of two evaluated patients treated with 0.3 mg/kg (FIG. 18A). An increased frequency of memory/effector CD4^{POS} and CD8^{POS} T cells was also observed (FIGS. 18B-18C) in three out of three evaluated patients. PBMCs stimulated with peptides derived from SARS-CoV-2 membrane (M), nucleocapsid (N), or spike (S) produced Th1 cytokines such as IFNg and IL-2 following CPI-006 treatment in one patient evaluated thus far (FIGS. 18D-18E).

### DISCUSSION

CPI-006 is a humanized FcgR binding-deficient anti-CD73 mAb that directly activates CD73^{POS} B cells, thereby inducing their trafficking to lymphoid tissues and promoting antibody production and differentiation into memory B cells. CPI-006 induces markers of B cell activation (CD69), maturation (CD138, CD28), and antigen presentation (CD86, CD83, MHC-II) in vitro along with a corresponding morphologic transformation into antibody secreting plasmablasts.

Studies in cancer patients demonstrated diversification of the BCR repertoire by stimulating the emergence and expansion of novel B cell clones. These findings indicate that CPI-006 can activate CD73^{POS} B cells to elicit antigen specific humoral and memory responses that display functional hallmarks associated with protective immunity. These effects are independent of CD73 enzymatic activity as addition of adenosine analogs or blockade of CD73 enzymatic activity alone has no effect on B cell activation in vitro. To our knowledge, CPI-006 is the only anti-CD73 antibody or small molecule inhibitor in development with the ability to directly activate B or T cells.

CD73 was originally characterized as a costimulatory molecule for T cells, but our results demonstrate that CPI-006 predominantly activates B cells.[11] Human CD73 is expressed on IgD^{POS}IgM^{DIM/NEG} naïve B cells, and CD27^{POS} memory B cells expressing IgG or IgA.[14] CPI-006 induces the expression of CD69, an activation marker that negatively regulates S1PR1 function, resulting in the prolonged retention of activated B cells in lymphoid organs and thymus.[27] This increased lymphoid residence time provides time to complete B cell activation and interact with CD4^{POS} T follicular helper cells to shape downstream immune responses. While B cell activation with CPI-006 is independent of adenosine blockade in vitro, the CD73 enzymatic blockade may be complementary in vivo as adenosine has been shown to restrict lymphocyte migration into lymph nodes in preclinical animal models.[28]

This study demonstrated that antibody titers to TS and RBD with neutralizing activity toward RBD increased within 7 days in patients treated with a single, low dose of CPI-006. The single, low doses evaluated in the study are noteworthy as the maximal concentrations of CPI-006 in plasma are modeled to exceed the 1 μg/ml threshold needed for maximal B cell activation yet low enough to be rapidly cleared. Although CPI-006 was delivered intravenously in this study, the effects seen with low doses indicate that alternative routes of delivery such as subcutaneous or intramuscular administration are feasible.

Most patients with COVID-19 become seropositive for IgG/IgM/IgA within 2-3 weeks following onset of symptoms.[29] However, all of the patients on this trial had low levels of anti-SARS-CoV2 antibodies at the time of hospitalization despite a wide range of duration of prior symptoms from 1-21 days. The lack of response in these patients may be related to host factors reducing the ability to mount a humoral response or other unknown immunosuppressive effects of viral infection. CPI-006 overcomes this apparent immunodeficiency as we observed robust anti-SARS-CoV-2 antibody responses induced by CPI-006 in patients with long POS and low pre-treatment titers. The kinetics following seroconversion are still being clarified, but others have reported that antibody titers in COVID-19 patients plateau approximately 6 days after seroconversion before steadily declining in the following weeks (IgM/IgA) to months (IgG).[29] Our results show that anti-SARS-CoV-2 titers continue to rise without plateauing in the weeks following treatment, demonstrating a robust and durable humoral response that improves clinical outcomes in COVID-19 patients. These effects also serve to reduce viral transmission and expand the pool of qualified convalescent plasma donors.

Temporary protection against SARS-CoV-2 can be imparted by circulating neutralizing antibodies, but the key to long term immunity lies in the production of antigen-specific memory B and T cells capable of recognizing and eliminating any potential re-infection. Indeed, CD4^{POS} and CD8^{POS} T cells able to cross-react with SARS-CoV-2 were detected in patients 6 years after recovering from the original 2003 SARS-CoV outbreak.[30] The increased frequency in memory B cells we observed following CPI-006 treatment in cancer patients has thus far been measured in a number of COVID-19 patients. Additional studies are underway to evaluate the antigen specificity of these memory B cells. An increase in the frequency of memory/effector T cells were also observed. Serial assessments of these populations will be required to determine the durability of immunological memory and whether a dose response emerges as we continue to escalate the CPI-006 dose. We note that imbalanced B and T cell responses have been reported in COVID-19 patients, including protective T cell responses without detectable seroconversion and, conversely, impaired CD4^{POS} T cell responses in critically ill patients with high IgG antibody titers. B cell recruitment and responses in peri-tumoral tertiary lymphoid structures has been associated with response to immunotherapy; thus, CPI-006 could help restore coordinated B and T cell responses in COVID-19 patients. [31-33]

Multiple viral and mRNA-based vaccines in development have demonstrated the potential to induce humoral and cellular responses to SARS-CoV-2 antigens in vaccinated normal subjects.[34, 35] While encouraging, rapidly diminishing antibody titers approximately two weeks after vaccination have necessitated a second booster vaccination in some instances.[34]

CPI-006 is being developed as a therapeutic, but it is important to note that it could also be used as a vaccine adjuvant to boost the titer, diversity, and duration of antibody responses and potentiate the development of long term immunity by generating memory B and effector T cells. This combination approach may be useful to enable successful immunization with a single vaccination and particularly effective in vulnerable populations such as immunosuppressed and elderly patients who do not typically respond well to vaccines.

The use of CPI-006 in COVID-19 patients has several advantages over other therapeutic approaches. CPI-006 may stimulate enhanced production of anti-viral antibodies in SARS-CoV-2 infected patients to shorten both the disease duration and time to viral clearance. Such antibody responses are also likely to be polyclonal, generating antibodies reacting with multiple viral antigenic determinants, not only on TS but other viral proteins as well, that would minimize the probability of developing resistance through natural selection of epitope loss variants. The increase in memory B and T cells can also provide longer lasting immunity and prevention of re-infection and transmission. Other approaches such as anti-viral drugs or passively administered monoclonal antibodies can be compromised by the evolution of viral mutations able to escape therapeutic targeting. Monoclonal antibodies against SARS-CoV-2 will also require significant quantities of infused immunoglobulin(s) and may need repeated infusions in order to maintain concentrations required for passive immunity. In contrast, low doses of CPI-006 can be immediately deployed in order to induce a diverse repertoire of high titer anti-viral antibodies in patients exposed to SARS-CoV-2, and other infectious disease. We note that diversifying the repertoire of antigen specific B cells would also facilitate the development of a broader cocktail of therapeutic mAbs when needed as these cells can be directly isolated, sequenced, and screened for antibodies targeting unique viral epitopes.[36]

These results demonstrate an atypically robust and durable antibody response following CPI-006 treatment, and are consistent with our hypothesized biological mechanism. We expect that longitudinal assessments of neutralizing antibody titers and β and T cell functional assays will demonstrate the contribution of CPI-006 to humoral and cellular immune responses.

The methods are described herein. Additional methods are set forth in the Supplemental Materials in Willingham et al, medRxiv, Sep. 15, 2020 at https://www.medrxiv.org/content/10.1101/2020.09.10.20191486v1 for additional details.

### Antibodies

CPI-006 was engineered by isolating VH and VL regions from the parental hybridoma generated by immunizing mice with human CD73 and screening for inhibition of CD73 activity. Humanization was performed by inserting CDRs isolated from the hybridoma into a human framework and the antibody was expressed as a human kappa/IgG1 antibody with the N297Q mutation introduced into the CH2 region to eliminate FcgR binding. Oleclumab was cloned using the VH and VL chain sequences published in WO 2016/075099 A1 application patent and was expressed as a human lambda/IgG1-TM antibody. Both antibodies were expressed in Expi-293 cells (Thermo Fisher Scientific) and purified by Protein A chromatography (HiTrap Protein A, GE Healthcare Life Sciences). For flow cytometry analysis, antibodies were labeled with AlexaFluor 647 (Life Technologies). Human IgG1 isotype control used for in vitro and vivo studies was purchased from Sigma-Aldrich and BioXCell, respectively. Fab fragments were prepared using the Pierce Fab Preparation kit (Thermo Fisher Scientific). Anti-CD73 antibody clone AD2 was purchased from Abcam.

### Affinity Measurements

Binding experiments were performed on Octet HTX at 25° C. Purified antibodies (2 μg/mL) were loaded onto Anti-Human IgG Fc Capture biosensors. Loaded sensors were dipped into a threefold dilution series of antigen (starting at 300 nM). Kinetic constants were calculated using a monovalent (1:1) binding model.

### Anti-SARS-CoV-2 Antibody ELISA Assays

ELISA was performed to measure the IgG, IgM, IgA antibody titer to the receptor-binding domain (RBD) of the spike protein and full-length spike trimer of the SARS-CoV-2 virus. Purified recombinant SARS-CoV-2 RBD and full-length spike protein were obtained from LakePharma. Briefly, ELISA plates were coated with RBD or spike protein (2 μg/mL in PBS) overnight at 4° C. then blocked with 3% BSA in PBS for 1 hr. at room temperature (RT) after three washes with PBST. Serial dilutions of serum were prepared in PBST containing 1% BSA, then dispensed to the wells of the coated microtiter plate and incubated for 2 hr at RT. After three washes, the bound antibody was detected using anti-human IgG-horseradish peroxidase (HRP) conjugated secondary antibody (1:3000, Sigma-Aldrich, A0170) or anti-human IgM HPR secondary antibody (1:3000, Sigma-Aldrich, A0420), or anti-human IgA HRP secondary antibody for 1 hr at RT. After three washes, the reaction was developed by the addition of the substrate o-phenylenediamine dihydrochloride (SigmaFast OPD) and stopped by HCl (2M). The absorbance at 490 nm (OD490) was measured using an Envision plate reader (PerkinElmer). Recovered COVID-19 patient serum was obtained from Sanguine Biosciences from recovered patients confirmed to have had a COVID-19 PCR+ result. All serum samples were treated to inactivate infectious virus by incubation at 56° C. for 30 mins. Healthy volunteer serum samples obtained from Stanford Blood Center during 2018 served as negative control. The titer cutoff value at OD490 is the mean plus 3 standard deviations of the negative controls. The endpoint titer is reported as the highest dilution of at least two before the OD490 decreases below the cut-off value. Data were analyzed using GraphPad Prism 7.

### RBD-ACE2 Blocking Assay

A MaxiSORP ELISA plate (Nunc) was pre-coated with human ACE2 protein (GenScript) at 100 ng per well in 50 μL of 100 mM carbonate-bicarbonate coating buffer (pH 9.6) overnight at 4° C., followed by blocking with OptEIA assay diluent (BD). Human sera were prepared using a 2.5-fold serial dilution starting at 1:5. The sera was diluted a further 2-fold by addition of an equal volume of HRP-conjugated RBD (Genscript) and the mixture incubated for 30 min at 37° C. in a final volume of 150 μL. The mixture (100 μL) was transferred to the wells of the precoated ELISA plate and incubated for 15 min at 37° C. Unbound HRP-RBD was removed by washing the plate four times with 260 μL of phosphate-buffered saline, 0.05% Tween-20. Bound antigen was detected by addition of 100 μL of the chromogenic substrate, 3,3′,5,5′-tetramethylbenzidine followed by incubation for 20 min at 22° C. The reaction was quenched by addition of 50 μL of 3 N HCl and the absorbance at 450 nm read using an EnVision plate reader (PerkinElmer). ID50 values were obtained by fitting the response-normalized data to a four-parameter logistic equation using GraphPad Prism version 8.4.3 for Windows, GraphPad Software, San Diego, California USA.

### Antigen Specific T Cell Peptivator Assay

Cryopreserved PBMCs from pre-2020 control donors or CPI-006 treated COVID-19 patients were thawed, washed, and incubated in RPMI 1640 (ATCC, Catalog #30-2001) plus 5% human serum (Sigma, Catalog #H4522-100ML) with Pen/Strep (Gibco, Catalog #15140122) for 24 hours with 600 nM SARS-CoV-2 peptides including S peptide (Miltenyi Biotec 130-126-700), M peptide (Miltenyi Biotec, Catalog #130-126-702), N peptide (Miltenyi Biotec, Catalog #130126-698) or control HIV peptides (JPT, Catalog #PM-HIV-CONB). Culture supernatants were collected and cytokines were assayed according to manufacturer's protocol using the V-PLEX Proinflammatory Panel 1 Human Kit (MSD, Catalog #15049D)

### Immunophenotyping of CPI-006 Treated Patient PBMCs

PBMCs from COVID-19 patients were isolated within 24 hr of blood collection and cryopreserved for flow cytometry. Expression of cell surface markers associated with B and T cell activation were assessed by flow cytometry using Fc blocking reagent (Miltenyi Biotech, Catalog #130-059-901) and antibodies directed to CD19 BV421 (Clone HIB19; BD Biosciences, Cat #562440), CD38 BV510 (Clone HB-7; BioLegend, Cat #356612), IgD FITC (Clone IA6-2; BD Biosciences, Cat #555778), CD73 PE (Clone AD2; BD Biosciences, Cat #550257), Mouse antihuman PD-1 PerCP 5.5 (clone EH12.1, BD Biosciences, Catalog #561273), CD3 PE-Cy7 (Clone UCHT1; BioLegend, Cat #300420), CD27 APC (Clone L128; BD Biosciences, Cat #337169), Mouse anti-human CD45RA APC-Fire 750 (clone HI100, BioLegend, Catalog #304152), Mouse anti-human CD8 BVD650 (clone SKI, BD Biosciences, Catalog #565289), Mouse anti-human CD4 PE-CF594 (clone SK3, BD Biosciences, Catalog #566914), Rat anti-human CXCR5 APC-R700 (clone RF8B2, BD Biosciences, Catalog #565191). Cryopreserved PBMCs from COVID-19 patients were analyzed at Corvus Pharmaceuticals using a CytoFLEX (Beckman Coulter). Fresh blood from cancer patients were analyzed at ICON using the same antibody clones. Flow data was analyzed using FlowJo v10.7.

### B Cell Receptor Analysis

Sequencing of the CDR3 regions of human variable chains was performed using the immunoSEQ® BCR Assay (Adaptive Biotechnologies, Seattle, WA). Genomic DNA was extracted from PBMCs and was amplified in a bias-controlled multiplex PCR, followed by high-throughput sequencing and the abundance of each unique BCR region was quantified.[37-39]

While various embodiments and aspects of the disclosure are shown and described herein, it will be obvious to those skilled in the art that such embodiments and aspects are provided by way of example only. Numerous variations, changes, and substitutions will now occur to those skilled in the art without departing from the disclosure. It should be understood that various alternatives to the embodiments of the disclosure described herein may be used. All publications, patents, and patent applications cited herein are hereby incorporated by reference in their entirety for all purposes.

### REFERENCES

(1) Wu, F., et al., A new coronavirus associated with human respiratory disease in China. Nature, 2020. 579(7798): p. 265-269. (2) Chan, J. F., et al., A familial cluster of pneumonia associated with the 2019 novel coronavirus indicating person-to-person transmission: a study of a family cluster. Lancet, 2020. 395(10223): p. 514-523. (3) Zhou, P., et al., A pneumonia outbreak associated with a new coronavirus of probable bat origin. Nature, 2020. 579(7798): p. 270-273. (4) Huang, C., et al., Clinical features of patients infected with 2019 novel coronavirus in Wuhan, China. Lancet, 2020. 395(10223): p. 497-506. (5) Du, L., et al., The spike protein of SARS-CoV—a target for vaccine and therapeutic development. Nat Rev Microbiol, 2009. 7(3): p. 226-36. (6) Wrapp, D., et al., Cryo-EM structure of the 2019-nCoV spike in the prefusion conformation. Science, 2020. 367(6483): p. 1260-1263. (7) Hoffmann, M., et al., SARS-CoV-2 Cell Entry Depends on ACE2 and TMPRSS2 and Is Blocked by a Clinically Proven Protease Inhibitor. Cell, 2020. 181(2): p. 271-280 e8. (8) Renn, A., et al., Fruitful Neutralizing Antibody Pipeline Brings Hope To Defeat SARS-Cov-2. Trends Pharmacol Sci, 2020. (9) Joyner, M. J., et al., Effect of Convalescent Plasma on Mortality among Hospitalized Patients with COVID-19: Initial Three-Month Experience. medRxiv, 2020. (10) Rasheed, A. M., et al., The therapeutic effectiveness of Convalescent plasma therapy on treating COVID-19 patients residing in respiratory care units in hospitals in Baghdad, Iraq. medRxiv, 2020: p. 2020.06.24.20121905. (11) Resta, R. and L. F. Thompson, T cell signalling through CD73. Cell Signal, 1997. 9(2): p. 131-9. (12) Resta, R., Y. Yamashita, and L. F. Thompson, Ecto-enzyme and signaling functions of lymphocyte CD73. Immunol Rev, 1998. 161: p. 95-109. (13) Thomson, L. F., et al., Production and characterization of monoclonal antibodies to the glycosyl phosphatidylinositol-anchored lymphocyte differentiation antigen ecto-5′-nucleotidase (CD73). Tissue Antigens, 1990. 35(1): p. 9-19. (14) Glass, D. R., et al., An Integrated Multi-omic Single-Cell Atlas of Human B Cell Identity. Immunity, 2020. 53(1): p. 217-232 e5. (15) Thompson, L. F., et al., Ecto-5′-nucleotidase expression during human B cell development. An explanation for the heterogeneity in B lymphocyte ecto-5′-nucleotidase activity in patients with hypogammaglobulinemia. J Immunol, 1986. 137(8): p. 2496-500. (16) Schena, F., et al., Dependence of immunoglobulin class switch recombination in B cells on vesicular release of ATP and CD73 ectonucleotidase activity. Cell Rep, 2013. 3(6): p. 1824-31. (17) Burger, R. M. and J. M. Lowenstein, 5′-Nucleotidase from smooth muscle of small intestine and from brain. Inhibition of nucleotides. Biochemistry, 1975. 14(11): p. 23626. (18) Luke, J. J., et al., Immunobiology, preliminary safety, and efficacy of CPI-006, an anti-CD73 antibody with immune modulating activity, in a phase 1 trial in advanced cancers. Journal of Clinical Oncology, 2019. 37(15_suppl): p. 2505-2505. (19) Geoghegan, J. C., et al., Inhibition of CD73 AMP hydrolysis by a therapeutic antibody with a dual, non-competitive mechanism of action. MAbs, 2016. 8(3): p. 454-67. (20) Airas, L., J. Niemela, and S. Jalkanen, CD73 engagement promotes lymphocyte binding to endothelial cells via a lymphocyte function-associated antigen-1-dependent mechanism. J Immunol, 2000. 165(10): p. 5411-7. (21) Airas, L., et al., Differential Regulation and Function of CD73, a Glycosyl-Phosphatidylinositol-linked 70-kD Adhesion Molecule, on Lymphocytes and Endothelial Cells. J Cell Biol, 1997. 136(2): p. 421-431. (22) de Lera Ruiz, M., Y. H. Lim, and J. Zheng, Adenosine A2A receptor as a drug discovery target. J Med Chem, 2014. 57(9): p. 3623-50. (23) Bystry, R. S., et al., B cells and professional APCs recruit regulatory T cells via CCL4. Nat Immunol, 2001. 2(12): p. 1126-32. (24) Schaniel, C., et al., Activated murine B lymphocytes and dendritic cells produce a novel CC chemokine which acts selectively on activated T cells. J Exp Med, 1998. 188(3): p. 451-63. (25) Takahashi, K., et al., CCL3 and CCL4 are biomarkers for B cell receptor pathway activation and prognostic serum markers in diffuse large B cell lymphoma. Br J Haematol, 2015. 171(5): p. 726-35. (26) Tan, C. W., et al., A SARS-CoV-2 surrogate virus neutralization test based on antibody-mediated blockage of ACE2-spike protein-protein interaction. Nat Biotechnol, 2020. (27) Shiow, L. R., et al., CD69 acts downstream of interferon-alpha/beta to inhibit S1P1 and lymphocyte egress from lymphoid organs. Nature, 2006. 440(7083): p. 540-4. (28) Takedachi, M., et al., CD73-generated adenosine restricts lymphocyte migration into draining lymph nodes. J Immunol, 2008. 180(9): p. 6288-96. (29) Long, Q. X., et al., Antibody responses to SARS-CoV-2 in patients with COVID-19. Nat Med, 2020. 26(6): p. 845-848. (30) Tang, F., et al., Lack of peripheral memory B cell responses in recovered patients with severe acute respiratory syndrome: a six-year follow-up study. J Immunol, 2011. 186(12): p. 7264-8. (31) Petitprez, F., et al., B cells are associated with survival and immunotherapy response in sarcoma. Nature, 2020. 577(7791): p. 556-560. (32) Cabrita, R., et al., Tertiary lymphoid structures improve immunotherapy and survival in melanoma. Nature, 2020. 577(7791): p. 561-565. (33) Helmink, B. A., et al., B cells and tertiary lymphoid structures promote immunotherapy response. Nature, 2020. 577(7791): p. 549-555. (34) Jackson, L. A., et al., An mRNA Vaccine against SARS-CoV-2—Preliminary Report. N Engl J Med, 2020. (35) Folegatti, P. M., et al., Safety and immunogenicity of the ChAdOx1 nCoV-19 vaccine against SARS-CoV-2: a preliminary report of a phase 1/2, single-blind, randomised controlled trial. Lancet, 2020. 396(10249): p. 467-478. (36) Zost, S. J., et al., Rapid isolation and profiling of a diverse panel of human monoclonal antibodies targeting the SARS-CoV-2 spike protein. Nat Med, 2020. (37) Robins, H. S., et al., Comprehensive assessment of T-cell receptor beta-chain diversity in alphabeta T cells. Blood, 2009. 114(19): p. 4099-107. (38) Robins, H., et al., Ultra-sensitive detection of rare T cell clones. J Immunol Methods, 2012. 375(1-2): p. 14-9. (39) Carlson, C. S., et al., Using synthetic templates to design an unbiased multiplex PCR assay. Nat Commun, 2013. 4: p. 2680.

1. A method of treating COVID-19 in a patient in need thereof, the method comprising administering to the patient an effective amount of an anti-CD73 antibody comprising an 1E9 CDR L1, an 1E9 CDR L2, an 1E9 CDR L3, an 1E9 CDR H1, an 1E9 CDR H2, and an 1E9 CDR H3.

2-4. (canceled)

5. A method of treating an infection or infectious disease in a patient in need thereof, the method comprising administering to the patient an effective amount of an anti-CD73 antibody comprising an 1E9 CDR L1, an 1E9 CDR L2, an 1E9 CDR L3, an 1E9 CDR H1, an 1E9 CDR H2, and an 1E9 CDR H3.

6. The method of claim 5, wherein the anti-CD73 antibody comprises an 1E9 CDR L1 having SEQ ID NO:1, an 1E9 CDR L2 having SEQ ID NO:2, an 1E9 CDR L3 having SEQ ID NO:3, an 1E9 CDR H1 having SEQ ID NO:4, an 1E9 CDR H2 having SEQ ID NO:5, and an 1E9 CDR H3 having SEQ ID NO:6.

7. The method of claim 5, wherein the anti-CD73 antibody comprises a light chain variable region having SEQ ID NO:8 and a heavy chain variable region having SEQ ID NO:7.

8. The method of claim 5, wherein the anti-CD73 antibody comprises a light chain having SEQ ID NO:10 and a heavy chain having SEQ ID NO:9.

9. The method of claim 5, wherein the infection or infectious disease is bacterial, viral, fungal, or parasitic.

10. The method of claim 5, wherein the infection or infectious disease is clonorchiasis, paragonimiasis, pinworms, hookworms, schistosomiasis, dysentery, infectious diarrhea, human avian influenza, hemorrhagic conjunctivitis, filariasis, kala-azar, Hydatid disease, hand foot and mouth disease, acute flaccid myelitis, anaplasmosis, anthrax, California serogroup virus disease, chikungunya virus disease, Eastern equine encephalitis virus disease, powassan virus disease, St. Louis encephalitis virus disease, West Nile virus disease, dengue fever, Western equine encephalitis virus disease, babesiosis, botulism, brucellosis, Campylobacteriosis, Candida auris, carbapenemase producing carbapenem-resistant enterobacteriaceae, chancroid, Chlamydia trachomatis infection, cholera, Clostridium difficile infection, Clostridium perfringens, coccidiodomycosis, syphilis, gonorrhea, COVID-19, cryptosporidiosis, cyclosporiasis, Dengue virus infection, diphtheria, Anaplasma phagocytophilum infection, Ehrlichia chaffeensis infection, Ehrlichia ewingii infection, giardiasis, gonorrhea, Haemophilus influenzae, Hansen's disease, hantavirus infection, non-hantavirus pulmonary syndrome, hemolytic uremic syndrome, hepatitis A, hepatitis B, hepatitis C, hepatitis E, HIV infection, influenza-associated pediatric mortality, pneumococcal disease, legionellosis, leptospirosis, listeriosis, Lyme disease, malaria, measles, meningococcal meningitis, mumps, measles, influenza A virus infection, pertussis, Bubonic plague, septicemic plague, pneumonic plague, polio, psittacosis, Q fever, rabies, rubella, a Salmonella bacterial infection, a Pseudomonas bacterial infection, a Mycobacterium bacterial infection, a Streptococcus bacterial infection, salmonellosis, severe acute syndrome-associated coronavirus disease, shiga toxin-producing Escherichia coli, shigellosis, smallpox, spotted fever rickettsiosis, streptococcal toxic shock syndrome, tetanus, toxic shock syndrome, trichinellosis, tuberculosis, tularemia, typhoid, paratyphoid, a Staphylococcus bacterial infection, a Nocardia bacterial infection, an Aspergillosis fungal infection, a Candida fungal infection, varicella, vibriosis, Crimean-Congo hemorrhagic fever virus, Ebola virus, yellow fever, Zika virus infection, Creutzfeldt-Jacob disease, herpes simplex-1, herpes simplex-2, genital herpes, herpes zoster, scarlet fever, hantavirus hemorrhagic fever, or human papillomavirus.

11-14. (canceled)

15. A method of producing an infectious disease antigen-binding antibody, the method comprising:
(i) administering to an infectious disease subject an effective amount of an anti-CD73 antibody comprising an 1E9 CDR L1, an 1E9 CDR L2, an 1E9 CDR L3, an 1E9 CDR H1, an 1E9 CDR H2, and an 1E9 CDR H3;
(ii) isolating from the infectious disease subject a B cell expressing an infectious disease antigen-binding antibody; and
(iii) expressing the gene encoding the infectious disease antigen-binding antibody,
thereby producing an infectious disease antigen-binding antibody.

16-24. (canceled)

25. A method of treating an infection or infectious disease in a subject in need thereof, the method comprising administering to the subject an effective amount of an infectious disease antigen-binding antibody formed by the method of claim 15.

26. The method of claim 25, wherein the infection or infectious disease is clonorchiasis, paragonimiasis, pinworms, hookworms, schistosomiasis, dysentery, infectious diarrhea, human avian influenza, hemorrhagic conjunctivitis, filariasis, kala-azar, Hydatid disease, hand foot and mouth disease, acute flaccid myelitis, anaplasmosis, anthrax, California serogroup virus disease, chikungunya virus disease, Eastern equine encephalitis virus disease, powassan virus disease, St. Louis encephalitis virus disease, West Nile virus disease, dengue fever, Western equine encephalitis virus disease, babesiosis, botulism, brucellosis, Campylobacteriosis, Candida auris, carbapenemase producing carbapenem-resistant enterobacteriaceae, chancroid, Chlamydia trachomatis infection, cholera, Clostridium difficile infection, Clostridium perfringens, coccidiodomycosis, syphilis, gonorrhea, COVID-19, cryptosporidiosis, cyclosporiasis, Dengue virus infection, diphtheria, Anaplasma phagocytophilum infection, Ehrlichia chaffeensis infection, Ehrlichia ewingii infection, giardiasis, gonorrhea, Haemophilus influenzae, Hansen's disease, hantavirus infection, non-hantavirus pulmonary syndrome, hemolytic uremic syndrome, hepatitis A, hepatitis B, hepatitis C, hepatitis E, HIV infection, influenza-associated pediatric mortality, pneumococcal disease, legionellosis, leptospirosis, listeriosis, Lyme disease, malaria, measles, meningococcal meningitis, mumps, measles, influenza A virus infection, pertussis, Bubonic plague, septicemic plague, pneumonic plague, polio, psittacosis, Q fever, rabies, rubella, a Salmonella bacterial infection, a Pseudomonas bacterial infection, a Mycobacterium bacterial infection, a Streptococcus bacterial infection, salmonellosis, severe acute syndrome-associated coronavirus disease, shiga toxin-producing Escherichia coli, shigellosis, smallpox, spotted fever rickettsiosis, streptococcal toxic shock syndrome, tetanus, toxic shock syndrome, trichinellosis, tuberculosis, tularemia, typhoid, paratyphoid, a Staphylococcus bacterial infection, a Nocardia bacterial infection, an Aspergillosis fungal infection, a Candida fungal infection, varicella, vibriosis, Crimean-Congo hemorrhagic fever virus, Ebola virus, yellow fever, Zika virus infection, Creutzfeldt-Jacob disease, herpes simplex-1, herpes simplex-2, genital herpes, herpes zoster, scarlet fever, hantavirus hemorrhagic fever, or human papillomavirus.

27-36. (canceled)

37. The method of claim 5, wherein the anti-CD73 antibody is administered at a half maximal effective concentration (EC₅₀) of at least 100 nM.

38. The method of claim 37, wherein the anti-CD73 antibody is administered at an EC₅₀ of about 137 nM or about 189 nM.

39. (canceled)

40. The method of claim 5, wherein the effective amount is from about 0.1 mg/kg to about 120 mg/kg.

41-45. (canceled)

46. The method of claim 5, wherein the anti-CD73 antibody is capable of binding a CD73 antigen with an equilibrium dissociation constant (K_{D}) from about 0.3 to about 25 nM.

47. The method of claim 46, wherein the anti-CD73 antibody is capable of binding a CD73 antigen with an equilibrium dissociation constant (K_{D}) of about 0.64 nM.

48. (canceled)

49. The method of claim 5, wherein the anti-CD73 antibody body is capable of binding a CD73 antigen at a pH from about 6.0 to about 7.0.

50-55. (canceled)

56. The method of claim 5, wherein the infection or infectious disease is viral.

57. The method of claim 5, wherein the infection or infectious disease a viral respiratory infection; a viral gastrointestinal infection; an exanthematous viral disease; a hepatic viral disease; a cutaneous viral disease; a viral hemorrhagic disease; or a neurologic viral disease.

58. The method of claim 5, wherein the infection or infectious disease severe acture respiratory syndrome, bronchiolitis, common cold, croup, influenza, penumonia, norovirus, rotavirus, adenovirus, astrovirus, measles, mumps, rubella, chickenpox, shingles, roseola, smallpox, fifth disease, chikungunya, hepatitis A, hepatitis B, hepatitis C, hepatitis D, hepatitis E, warts, genital warts, herpes simplex, HSV-1, HSV-2, genital herpes, molluscum contagiosum, Ebola, lassa fever, dengue fever, yellow fever, marburg hemorrhagic fever, hemorrhagic conjunctivitis, Crimean-Congo hemorrhagic fever, polio, viral meningitis, viral encephalitis, rabies, a Zika viral infection, a West Nile viral infection, Epstein Barr virus, cytomegalovirus, malaria, or tuberculosis.

59. The method of claim 5, further comprising administering an effective amount of antiviral agent, an antiviral vaccine, or a combination thereof.


SEQUENCE LISTING (12 sequences)

SEQ ID NO: 1 - protein, 15 aa, Artificial sequence
  Synthetic polypeptide
  misc_feature: CPI-006 CDR L1
RASKNVSTSGYSYMH

SEQ ID NO: 2 - protein, 7 aa, Artificial sequence
  Synthetic polypeptide
  misc_feature: CPI-006 CDR L2
LASNLES

SEQ ID NO: 3 - protein, 9 aa, Artificial sequence
  Synthetic polypeptide
  misc_feature: CPI-006 CDR L3
QHSRELPFT

SEQ ID NO: 4 - protein, 10 aa, Artificial sequence
  Synthetic polypeptide
  misc_feature: CPI-006 CDR H1
GYTFTSYWIT

SEQ ID NO: 5 - protein, 14 aa, Artificial sequence
  Synthetic polypeptide
  misc_feature: CPI-006 CDR H2
PGSGNTNYNEKFKT

SEQ ID NO: 6 - protein, 13 aa, Artificial sequence
  Synthetic polypeptide
  misc_feature: CPI-006 CDR H3
EGGLTTEDYALDY

SEQ ID NO: 7 - protein, 120 aa, Artificial sequence
  Synthetic polypeptide
  misc_feature: CPI-006 heavy chain
QVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTV

SEQ ID NO: 8 - protein, 111 aa, Artificial sequence
  Synthetic polypeptide
  misc_feature: CPI-006 light chain
EIVLTQSPATLSLSPGERATLSCRASKNVSTSGYSYMHWYQQKPGQAPRLLIYLASNLESGIPPRFSGSGYGTDFTLTINNIESEDAAYYFCQHSRELPFTFGQGTKVEIK

SEQ ID NO: 9 - protein, 471 aa, Artificial sequence
  Synthetic polypeptide
  misc_feature: CPX-006_HC
MEWSWVFLFFLSVTTGVHSQVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 238 aa, Artificial sequence
  Synthetic polypeptide
  misc_feature: CPX-006_LC
MSVPTQVLGLLLLWLTDARCEIVLTQSPATLSLSPGERATLSCRASKNVSTSGYSYMHWYQQKPGQAPRLLIYLASNLESGIPPRFSGSGYGTDFTLTINNIESEDAAYYFCQHSRELPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 111 aa, Artificial sequence
  Synthetic polypeptide
DIVLTQSPASLAVSLGQRATISCRASKNVSTSGYSYMHWYQQKPGQPPKLLIYLASNLESGVPTRFSGSGSGTDFTLNIHPVEEEDAATYYCQHSRELPFTFGSGTKLEIK

SEQ ID NO: 12 - protein, 122 aa, Artificial sequence
  Synthetic polypeptide
QVQLQQPGAELVKPGASVKMSCKASGYTFTSYWITWVKQRPGQGLEWIGDIYPGSGNTNYNEKFKTKATLTVDTSSSTAYMQLSSLTSEDSAVYYCAKEGGLTTEDYALDYWGQGTLVTVSS